(19) United States
(12) Patent Application Publication
(10) Pub. No.: US 2024/0082221 A1
(43) Pub. Date: Mar. 14, 2024

(54) COMPOSITIONS AND METHODS FOR THE IDENTIFICATION OF COMPOUNDS THAT PROTECT AGAINST LIPOFUSCIN CYTOTOXICITY

(71) Applicant: Cornell University, Ithaca, NY (US)

(72) Inventor: Marcelo M. Nociari, Summit, NJ (US)

(73) Assignee: Cornell University, Ithaca, NY (US)

(21) Appl. No.: 18/273,678

(22) PCT Filed: Jan. 21, 2022
(86) PCT No.: PCT/US2022/013276

§ 371 Date: Jul. 21, 2023

Related U.S. Application Data
(60) Provisional application No. 63/140,533, filed on Jan. 22, 2021.

(51) Int. Cl. A61K 31/427; A61K 31/17; A61K 31/4545; A61K 31/47; A61K 31/4985; A61K 31/506; A61K 31/635; A61K 45/06; A61P 27/02; G01N 33/50

(52) U.S. Cl. CPC A61K 31/427; A61K 31/17; A61K 31/4545; A61K 31/47; A61K 31/4985; A61K 31/506; A61K 31/635; A61K 45/06; A61P 27/02; G01N 33/5038; G01N 33/5044

(57) ABSTRACT

The present disclosure provides compositions and methods for treating eye diseases (e.g., retinopathies), and more particularly, eye diseases associated with cytotoxic lipofuscin-associated cytotoxicity in retinal cells.

### CROSS-REFERENCE TO RELATED APPLICATIONS

This application claims the benefit of and priority to U.S. Provisional Patent Application No. 63/140,533 filed Jan. 22, 2021, the entire contents of which is incorporated herein by reference.

### STATEMENT OF GOVERNMENT SUPPORT

This invention was made with government support under EY027422-04 awarded by the National Institutes of Health/National Eye Institute. The government has certain rights in the invention.

### TECHNICAL FIELD

The present technology relates generally to compositions and methods for treating eye diseases (e.g., retinopathies), and more particularly, eye diseases associated with cytotoxic lipofuscin-associated cytotoxicity in retinal cells.

### BACKGROUND

The following description of the background of the present technology is provided simply as an aid in understanding the present technology and is not admitted to describe or constitute prior art to the present technology.

Retinal pigment epithelium (RPE) cell-death is the primary cause of geographic atrophy (GA) in retinas with Stargardt and dry-AMD, the most prevalent and incurable genetic and age-related blinding disorders among young and old, respectively. Lipofuscin (LF) is a fine yellow-brown pigment composed of indigestible material that is believed to be remnants after lysosomal digestion. LF is mostly composed of dimers of retinaldehydes known as lipid bisretinoids, and small amounts of carbohydrates, oxidized proteins and metals. Accumulation of LF in retinal cells causes retinal toxicity, which is associated with conditions like macular degeneration, a degenerative disease of the eye, and Stargardt disease. Yet, the mechanisms and extent by which LF contributes to the degeneration is unclear in part because all attempts at targeting its cytotoxic effects have failed to maintain the RPE's viability and stop the retina's decay. Accordingly, there is an urgent need for novel molecular targets to treat GA secondary to Stargardt and dry-AMD.

### SUMMARY OF THE PRESENT TECHNOLOGY

In one aspect, the present disclosure provides a method for preventing or treating an eye disease associated with retinal cell lipofuscin-associated cytotoxicity in a subject in need thereof comprising administering to the subject an effective amount of at least one therapeutic agent selected from the group consisting of dabrafenib, necrosulfonamide (NSA), arimoclomol, a Kinase Inhibiting RNase Attenuator (KIRA) compound, salubrinal, SAL003 and any pharmaceutically acceptable salt thereof, wherein the eye disease associated with retinal cell lipofuscin-associated cytotoxicity is autosomal recessive retinitis pigmentosa (RP), Stargardt disease (STGD), Best disease (BD), cone-rod dystrophy, or ABCA4 mutant Age-Related Macular Degeneration (AMD). Examples of KIRA compounds include, but are not limited to, KIRA3, KIRA6, KIRA7, or KIRA8. In some embodiments, the subject comprises a mutation in ABCA4 and/or RDH12. The mutation in ABCA4 and/or RDH12 may be homozygous or heterozygous. Additionally or alternatively, in some embodiments of the methods disclosed herein, administration of the effective amount of the at least one therapeutic agent prevents exacerbation of lipofuscin-associated cytotoxicity in retinal cells in the subject.

In another aspect, the present disclosure provides a method for preventing or treating an ABCA4 mutant eye disease associated with retinal cell lipofuscin-associated cytotoxicity in a subject in need thereof comprising administering to the subject an effective amount of Necrostatin 7 (Nec7) or a pharmaceutically acceptable salt thereof, wherein the ABCA4 mutant eye disease associated with retinal cell lipofuscin-associated cytotoxicity is autosomal recessive retinitis pigmentosa (RP), cone-rod dystrophy, or Age-Related Macular Degeneration (AMD). In some embodiments, administration of the effective amount of Nec7 or pharmaceutically acceptable salt thereof prevents exacerbation of lipofuscin-associated cytotoxicity in retinal cells in the subject.

In any and all embodiments of the methods disclosed herein, the eye disease is genetic, non-genetic, or associated with aging. In some embodiments of the methods disclosed herein, the AMD is dry AMD. In other embodiments of the methods disclosed herein, the cone-rod dystrophy is autosomal recessive cone-rod dystrophy.

Additionally or alternatively, in some embodiments of the methods disclosed herein, the subject harbors at least one ABCA4 mutation selected from the group consisting of ABCA4 D2177N, ABCA4 G1961E, ABCA4 G863A, ABCA4 1847delA, ABCA4 L541P, ABCA4 T2028I, ABCA4 N247I, ABCA4 E1122K, ABCA4 W499*, ABCA4 A1773V, ABCA4 H55R, ABCA4 A1038V, ABCA4 IVS30+1G→T, ABCA4 IVS40+5G→A, ABCA4 IVS14+1G→C, and ABCA4 F1440del1cT. In any and all embodiments of the methods disclosed herein, the subject harbors at least one RDH12 mutation selected from the group consisting of RDH12 G127*, RDH12 Q189*, RDH12 Y226C, RDH12 A269Gfs*, RDH12 L274P, RDH12 R65*, RDH12 H151D, RDH12 T155I, RDH12 V41L, RDH12 R314W and RDH12 V146D. cone-rod dystrophy.

Additionally or alternatively, in some embodiments of the methods disclosed herein, the at least one therapeutic agent of the present technology (e.g., dabrafenib, NSA, arimoclomol, the KIRA compound, salubrinal, SAL003, Nec7, or the pharmaceutically acceptable salt thereof) reduces or eliminates lipofuscin bisretinoid (LB) lipid-induced phosphorylation and/or polymerization of MLKL. In any and all embodiments of the methods disclosed herein, the at least one therapeutic agent of the present technology (e.g., dabrafenib, NSA, arimoclomol, the KIRA compound, salubrinal, SAL003, Nec7, or the pharmaceutically acceptable salt thereof) reverses LB lipid-induced translocation of phosphorylated MLKL (pMLKL) to plasma membraned in retinal pigment epithelium cells. In certain embodiments, the LB lipids are selected from the group consisting of N-retinylidene-N-retinylethanolamine (A2E), an A2E isomer, an oxidized derivative of A2E, and all-trans-retinal dimers (ATRD).

In any of the preceding embodiments of the methods disclosed herein, the at least one therapeutic agent of the present technology (e.g., dabrafenib, NSA, arimoclomol, the KIRA compound, salubrinal, SAL003, Nec7, or the pharmaceutically acceptable salt thereof) reduces mRNA or protein levels of one or more genes associated with retinal degeneration, inflammation/angiogenesis, ER-stress and/or necroptosis. Examples of genes associated with retinal degeneration, inflammation/angiogenesis, ER-stress and/or necroptosis include, but are not limited to, EDN2, FGF2, GFAP, SERP, VEGF, CXCL15, XBP1s, SCAND1, CEBPA and HMGA. Additionally or alternatively, in some embodiments of the methods disclosed herein, the at least one therapeutic agent of the present technology (e.g., dabrafenib, NSA, arimoclomol, the KIRA compound, salubrinal, SAL003, Nec7, or the pharmaceutically acceptable salt thereof) inhibits or mitigates lipofuscin-induced necroptosis and/or reduces infiltration of activated microglia/macrophage in retinal pigment epithelium cells.

In any and all embodiments of the methods disclosed herein, the at least one therapeutic agent of the present technology (e.g., dabrafenib, NSA, arimoclomol, the KIRA compound, salubrinal, SAL003, Nec7, or the pharmaceutically acceptable salt thereof) is administered via topical, intravitreous, intraocular, subretinal, or subscleral administration. Additionally or alternatively, in some embodiments of the methods disclosed herein, the at least one therapeutic agent of the present technology (e.g., dabrafenib, NSA, arimoclomol, the KIRA compound, salubrinal, SAL003, Nec7, or the pharmaceutically acceptable salt thereof) is conjugated to an agent that targets retinal pigment epithelium cells. Examples of agents that target retinal pigment epithelium cells include, but are not limited to, tamoxifen, chloroquine (CQ)/hydroxychloroquine (HCQ), ethambutol (EMB), or sodium iodate (NaIO₃). Other examples of RPE targeting agents are described in Crisóstomo S, Vieira L, Cardigos J (2019) Retina:23-28; Michaelides M (2011) Arch Ophthalmol 129(1):30; Tsai R K, He M S, Chen Z Y, Wu W C, Wu W S (2011) Mol Vis 17(June):1564-1576; MacHalińska A, et al. (2010) Neurochem Res 35(11):1819-1827; Tsang S H, Sharma T (2018) Drug-Induced Retinal Toxicity. Atlas of Inherited Retinal Diseases, eds Tsang S H, Sharma T (Springer International Publishing, Cham), pp 227-232.

### BRIEF DESCRIPTION OF THE DRAWINGS

FIGS. 1A-1E demonstrate an uninterrupted increase in the content of LF-granules per RPE with aging. FIG. 1A: Autofluorescence images of flat-mounted RPE eyecups from young and old WT and DKO mice. 10× individual fields were stitched together and displayed in the following orientation: dorsal-top; ventral-bottom; nasal-right; temporal-left, bar=1 mm. Inserts show high-magnification views of RPE from central-equatorial areas, where yellow corresponds to LF-granules, red to ZO1 borders and blue to nuclei stained with Hoechst, bars=10 μm. FIG. 1B: LF fluorescence per RPE quantified by 63× microscopy. Borders were visualized with phalloidin and individual cells were manually selected with ImageJ. Each dot is the integrated auto-fluorescence/cell of 8 months and 33 months WT (n=5); 3 months DKO (n=4); 8 months DKO (n=6); 13 months DKO (n=4); 26 months DKO (n=5) RPE in central retina. The LF content in 3 month old DKO was higher than in 33 months WT (p<0.01). Also, LF/cell was significantly different between all DKO age groups (WT 8MS vs WT 33MS, p<0.05; DKO 3MS vs WT 33MS, p<0.01; p<0.001; DKO 8MS/DKO 13MS/DKO 26MS vs WT 33MS, p<0.0001). FIG. 1C: 63× image of lipofuscin in RPE layer and lipofuscin did not drop at 600d and 700d. FIG. 1D: 63× image of phalloidin cytoskeleton (red) and LF-granules (yellow) to see architectural abnormalities linked to LF buildup, bars=10 μm. FIG. 1E: HPLC quantitation of the content of A2E in the RPE of DKO mice at different ages. Each dot is the content in one eye. Bars represent the medians per age group.

FIGS. 2A-2H demonstrate degenerative changes in retinas with LF buildup. FIG. 2A: Sizes (μm²) of central retina's RPE. Each dot is the area/cell in 8 (n=5) and 27 (n=5) month WT and 8 (n=6) and 23 (n=5) month DKO animals. The oldest DKO group exhibited significantly enlarged RPE compared to all other groups (*p<0.01 by unpaired t test with Prism7). FIG. 2B: Number of RPE nuclei counted every 0.1 mm intervals and plotted as function of distance from ONH in 23 month old DKO (n=10) and 27 month old WT retinas (n=8). Mean values (±SEM) were significantly different for each point (DKO vs WT, p<0.05 by multiple t test with Prism7). FIG. 2C: ONL thicknesses measured every 0.1 mm intervals from the ONH along the vertical axis in DKO (n=5) and WT control eyes (n=7) at 27-months of age. Means (±SEM) were significantly different for each position (DKO vs WT, p<0.05 by multiple t test with Prism7). FIG. 2D: Representative fluorescence microscopy of cryosections from 30 month-old WT (n=3) and DKO (n=3) mice. DKO neuroretinas depicted abundant infiltration with ˜1-3 μm LF particles, 20 μm scale. FIG. 2E: Left image is 20 month old melanin-bleached paraffin-embedded cross-section of DKO showing the ˜1-3 μm LF infiltrates strongly positive for Iba1. Right image is a bright field/fluorescence overlay of 10 months old DKO showing ˜1-3 μm particles positive for rhodopsin (red) and negative for melanin (scale bar=20 μm). FIG. 2F: Cross-section of 800d DKO showing sloughed RPEs (arrows) migrating into the neural retina. FIG. 2G: Maximum projection of neural retina flat mount and Z-stack sections, obtained by confocal microscopy, showing migratory RPEs inserted at different depths into the photoreceptor layer. Scale bar=20 μm. FIG. 2H: H&E on paraffin-embedded cross-sections showing migration and multilayering in the RPE of DKOs. Migratory RPE contain melanin and measure ˜5-10 μm. DKO showed damage to the ONL overlying RPE with migratory/proliferative behavior.

FIGS. 2I-2J show degenerative changes in retinas with LF buildup. FIG. 2I: Representative microscope picture shows that microglia cell appeared in outer segment of DKO mouse eye is CD11b (red) and IBA1 (green) positive, it also loads with LB (white). FIG. 2J: RPE cells from different aged DKO mice show the accumulated A2E increase until 200 days, followed by less A2E accumulation as determined by HPLC.

FIGS. 3A-3B demonstrate that light-independent LF cytotoxicity is a major contributor to the degeneration of pigmented retinas. FIG. 3A: Loss of photoreceptors and RPEs between months 2 and 12 of age in animals reared in complete darkness versus 12 hrs light/dark cycles (n=6 per illumination, age and genotype condition). Red and blue correspond to ONL thickness, while orange and grey circles are RPE nuclei number, in 2 and 12 months old mice, respectively. Only DKOs showed thinning of ONL and loss of RPE (p<0.01), by two way ANOVA, during that period and were similar between light cycled and dark reared mice. FIG. 3B: Autofluorescence of eyecups from 12 month old DKO and WT raised under dark or cyclic illumination. Images were stitched together from individual fields, taken with a 5× objective (λexc=430 nm, λemm=610 nm) are oriented: dorsal-top; ventral-bottom; nasal-right; temporal-left. Scale bar=1 mm.

FIGS. 4A-4I demonstrate that light-independent LF cytotoxicity causes atypical necroptosis. FIG. 4A: Cell-death assay to study LF's dark toxicity. 90% confluent ARPE-19 or hfRPE were incubated overnight in serum free media supplemented with indicated lipid bisretinoids (LB) concentration. Incorporated autofluorescence localized within lamp2 lysosomes. Viability was assessed at 24 hrs by AlamarBlue®, or microscopy with DRAQ7/NUC405. FIG. 4B: Real time monitoring of necrotic (red is DRAQ7=plasma membrane leakage) and apoptotic (blue is NUC405=caspase 3 activation) cell-death by automated fluorescence microscopy. FIG. 4C: Neutralization of detergent activity does not protect against A2E. 10 μM methyl-βCD counteracted 500 μM TRITON-X100 (*p<0.001 in A2E, TRITON, A2E with MBCD compared to the non-treated cells. p values were determined by t test with Prism7.0 but did not affect the cytotoxicity of 20 μM A2E. FIG. 4D: Inhibition of effector cascades of programed necrosis. Dose-dependent protection with NSA (p<0.01) but not with pan-caspase, gasdermin-D nor RIPK3 inhibitors (n=4). FIG. 4E: IP with anti-MLKL showed significant increased kinase activity only in pulldowns from cells with accumulated A2E (p<0.01), suggesting the formation of a necrosome. FIG. 4F: Western blot with anti-Ser358 phospho-MLKL showing dose-dependent phosphorylation and polymerization of human MLKL. FIG. 4G: Protection by necrostatins. Necrostatin 7 (Nec7) (p<0.01), but not the anti-RIPK1 necrostatins: 1, is and 5 shielded against A2E cytotoxicity (n=3). FIG. 4H: Western blot showing Nec7 prevents phosphorylation and polymerization of MLKL by A2E. FIG. 4I: Fluorescence image of ARPE-19 monolayers showing A2E and ATRD inducing the translocation of pMLKL into plasma membranes which was blocked by Nec7.

FIGS. 4J-4T show light-independent LF cytotoxicity. FIG. 4J: Viability assay used to test toxicity of A2E 20 μM and ATRD 80 μM in ARPE199 cultures with different cell numbers. Fully confluent ARPE19 cells are more resistant to LB cell death. FIG. 4K: Increased cell confluency affects the amount of A2E taken into the cells. FIG. 4L: 20 μM A2E was loaded to the ARPE19 cells with different confluency for 24 hours. The A2E/cell amounts in the culture cells were comparable to the lipofuscin amount in RPE cells in DKO 800 day old mice. FIG. 4M: Comparison of ARPE19 and hfRPE cell survival to A2E treatment. The hfRPE cells are more resistant to A2E at 20 μM, 30 μM and 45 μM than ARPE19 cells (p<0.05, t test). FIG. 4N: Light-independent LF cytotoxicity in hfRPE compared with ARPE19. FIG. 4O: Necroptosis cascades induced by viruses, toll-like agonists, and TNF shows the pathway significantly depends on the nature of the necroptotic stimulus. FIG. 4P: Dose dependent induction of induction of phosphorylation and polymerization of MLKL with ATRD. FIG. 4Q: phosphorylation and polymerization of MLKL was not prevented by Nec1 nor GSL'872, but was abrogated by Nec7 and not by Nec1 (FIG. 4R). FIG. 4S: Analysis by RNAseq of the expression of the different isoforms of RIPK1, 2, 3 and 4 in ARPE19 cells. FIG. 4T: WB analyzing the activation of pMLKL, RIPK1, RIPK3 in HT29 undergoing cell death by treatment with A2E, ATRD or STZ.

FIG. 4U: Western blots showing that Nec7, but not Nec1, prevents MLKL phosphorylation/polymerization induced by lipofuscin materials. FIG. 4V: Melanin does not affect fluorescence quantification. Lysis buffer or RPE lysates from WT C57BL6, obtained as described in M&M, were spiked with 300 pmoles of A2E per ml and 430 nm/600 mn fluorescence was used for the quantification. FIG. 4W: Dose dependent cell death in ARPE19 cells exposed to A2E, ATRD, and ATR. FIG. 4X: Pre-treatment with 33 μM Nec1, Nec1s, or Nec7 did not block cell death by ATR. FIG. 4Y: Western blot with anti-phospho-MLKL and GAPDH shows ATR does not change the phosphorylated status of MLKL.

FIGS. 5A-5E demonstrate that LF-necroptosis does not involve oxidative-stress but ER− stress. FIG. 5A: Antioxidants such as Trolox, NAC, L-Cys, Vit-C, BHA and TMB did not rescue the ARPE-19 from different amounts of A2E. FIG. 5B: RNAseq/IPA analysis revealed that the protective effect of Nec7 mainly implicated a reduction of the unfolded protein response (UPR) pathway. FIG. 5C: View of canonical UPR cascade showing multiple mRNAs within the IRE1α and PERK pathways downregulated by Nec7. FIG. 5D: IPA predicted survival effects of downregulating the UPR with Nec7.

FIG. 5E demonstrates that Nec7 neutralizes the effect of A2E as evident from the close clustering of Ctr-Nec7 and A2E-Nec7 in both, heat-diagram and PCA analysis.

FIGS. 6A-6N demonstrate that LF triggers ER-stress, and inhibitors of IRE1α block necroptosis. FIG. 6A: Western blot showing p-eIF2a, ATF4 and BiP/GRP78 were upregulated by LF without illumination. FIG. 6B: ATF4 mRNA induction by dark A2E 25 M and 80 μM ATRD detected by qPCR (n=2). Dose (FIG. 6C) and kinetics (FIG. 6D) of induction of XBP1s, with A2E and ATRD, detected by qPCR (n=3). FIG. 6E: Agarose gel confirmation of XBP1 splicing in both ARPE-19 and primary hfRPE cells (n=2). FIG. 6F: Western Blot showing cleavage of ATF6 in cells with accumulated A2E. FIG. 6G: qPCR showing that Nec7 blocks XBP1s (IRE11α branch). FIG. 6H: Western Blot showing that Nec7 also blocks CHOP (downstream of PERK), and neither Nec1 nor GSK'872 effected the UPR, which is consistent with their lack of protection against necroptosis. FIG. 6I: Viability of ARPE-19 cells to LF after individual knock down of ER-stress sensor/effectors with shRNAs. Only IRE1α knockdown conferred protection against A2E and ATRD (p<0.05, n=3). FIG. 6J: Selective inhibition of IRE1α kinase and/or RNAse activation had no protective effect against LF, but drugs that block IRE1α dimerization (KIRAs) increased survival. FIG. 6K: Western blot showing that the IRE1α inhibitor, KIRA6 prevents phosphorylation and polymerization of MLKL induced by LF. FIG. 6L: Immunostaining of ARPE19 cells accumulating LF and treated with KIRA6 show inhibition of pMLKL plasma membrane translocation. FIG. 6M: Effectiveness of KIRA6 and Nec7 to promote survival to LF in hfRPE. FIG. 6N: qPCR confirming the induction by LF of XBP1 splicing and its prevention with Nec7 in hfRPE.

FIG. 6O demonstrates that antioxidants cannot prevent UPR induced by LF. WB showing that light-independent phosphorylation of IRE1α induced by LF, proceeded unaffected in the presence of NAC

FIGS. 7A-7F show ER-stress and necroptosis in DKO retinas. FIG. 7A: Flat mounted RPE-eyecups, immuno-stained with anti-XBP1s (red) and nuclear DAPI (blue) in the central-equatorial RPE from DKOs aged as indicated in the figure. XBP1s was negligible in old WT but detectable in 2 month old DKO and became stronger with aging. Bars=20 m (n=3 per group). FIG. 7B: ER-stress monitored with anti phospho-Ser345-HLKL (red) and nuclear DAPI (blue) show age-related increase of labeling which localized to plasma membranes in the oldest group (n=3), scale bars=20 μm. FIG. 7C: Desmelanized paraffin cross section showing XBP1s (green) and pMLKL (red) co-expression on the RPE layer and in small ˜1-3 μm Iba1+ cells. FIG. 7D: Iba1+ cells in subretinal space coexpressing Iba1(red)/XBP1s (green) or Iba1(red)/MLKL (green), scale bar=20 μm. FIG. 7E: Panoramic view of a large area of the neural retina with large “flecks”. Neural retina flat mounts were immune stained with anti-pMLKL (red). A notable feature was the halo of pMLKL in the ONL layer surrounding the RPE infiltrations. FIG. 7F: is a zoom view of the square region indicated in FIG. 7E, maximum projected on the z-axes show that the necroptosis signal spread in all directions around the invading RPE fragment.

FIGS. 7G-7I show ER-stress and necroptosis in RPE cells. FIG. 7G: Exemplary immunofluorescence confocal image showing colocalization of p-HLKL (red), XBP1s (green) and LB (white) on RPE flat mount samples from 700 days mice (n=3). Bar=20 μm. FIG. 7H: Single 1 μl intraocular injection of Nec7 decreases pMLKL levels in retinas as shown in RPE flat mounts from treated DKOs. FIG. 7I: Mean fluorescence intensity (MFI) plot of pMLKL measured every 0.1-mm intervals and plotted as function of distance from ONH in superior hemiretina, of vehicle and Nec7 treated eyes of 700 day old DKOs; (quantified by Image J, **p<0.01 for Mock vs KIRA6, n=3; determined by two-way ANOVA in GraphPad Prism).

FIG. 7J: Microglia/macrophages attached to RPE-flat mounted eyecups from 20 months old DKO retinas stained positive for phospho-MLKL. FIG. 7K: Phospho-MLKL staining (red) in a zone of RPE rich in lipofuscin (yellow) and with intense migratory activity.

FIG. 7L: Single intravitreal injection of Nec7 but not Nec1 eliminated phospho-MLKL staining in 20 months-old DKO retinas (n=4). FIG. 7M: Representative neural retina flat mount, showing the reduction of phospho-MLKL in the photoreceptor layer after receiving intraocular Nec7 1 week earlier. FIG. 7N: Subretinal infiltration of CD11b cells on RPE-flat mounts in 18 to 20-month-old DKOs that received 1 week earlier 2 μl intravitreal injections of vehicle (control) or Nec7, bar=20 μm.

FIGS. 8A-8E demonstrate that IRE1α inhibitors reduce inflammation and necroptosis in retinas with LF. FIG. 8A: Intravitreal injection of KIRA6 reduces ER-stress in DKO retinas. Center to periphery images, along the vertical axe, of RPE flat mounts from right (OD) and left (OS) eyes of 17 month old DKO treated with 1 μl of vehicle (Mock) or KIRA6, respectively. Immunostaining for XBP1s is depicted in green. Bars=20 μm. The inserts are a magnified view of central retina's RPE in those eyes. Bars=20 μm. Plots of mean fluorescence intensity (MFI) of XBP1s (green) immunostaining measured every 0.1-mm intervals and plotted as function of distance from ONH in superior hemiretina, of vehicle and KIRA6 treated eyes of 600 days old DKOs; (quantified by Image J, **p<0.01 for Mock vs KIRA6, n=3; determined by two-way ANOVA in GraphPad Prism). FIG. 8B: Center to periphery images, along the vertical axe, of RPE flat mounts from right (control) and left (treated) eyes of 512 day old DKOs. Mice received a single 1 μl intravitreal injection of vehicle or KIRA6 per eye. The pMLKL marker of necroptosis (red) was dramatically reduced (n=3). Bars=20 μm. The inserts are a magnified view of central retina's RPE in those eyes, scale 20 μm. Plots of mean fluorescence intensity (MFI) of pMLKL (red) immunostainings measured every 0.1-mm intervals and plotted as function of distance from ONH in superior hemiretina, of vehicle and KIRA6 treated eyes of 512 days old DKOs; (quantified by Image J, **p<0.01 for Mock vs KIRA6, n=3; determined by two way ANOVA in GraphPad Prism). FIG. 8C: Dot-plot representing the percentage of XBP1s/pMLKL double positive cells in cryosections of 600 day DKO. Top panel is OD-vehicle and bottom panel is OS-KIRA6 treated. FIG. 8D: KIRA6 reduces the number of Iba-1⁺ microglia/macrophages infiltrating the outer retina in 800d DKO (n=3). FIG. 8E: qPCR showing normalization with KIRA6 of the transcripts upregulated during photoreceptor degeneration. FIG. 8F shows a comparison of multiple markers of ongoing retinal degeneration in WT and DKO mice as detected by qPCR.

FIG. 9A shows an exemplary model explaining the protection by Salubrinal (and SAL003) against retinal lipofuscin. FIG. 9B shows an exemplary mechanism of action by which the compositions of the present technology protect against light-independent lipofuscin cytotoxicity. Lipofuscin forms solid crystals that when in high amounts punch the lysosomal membranes and causes LMP. The release of lysosomal enzymes triggers the formation of an atypical necrosome that phosphorylates MLKL, promoting its oligomerization and membranes translocation. Phospho-HLKL destabilizes the membrane of lysosomes promoting more LMP. When the levels of phospho-MLKL in plasma membrane become intolerable, the cell undergoes necroptosis.

FIG. 10 shows antibodies used in the Examples described herein.

FIG. 11 shows primer sequences used in the Examples described herein.

FIG. 12 shows chemical inhibitors used in the Examples described herein.

FIG. 13 shows antibodies and fluorescent probes used in the Examples described herein.

FIG. 14A: In the absence of illumination cellular ROS (red) were detected in the mitochondria of ARPE19 but not in lipofuscin granules (green). Only, after blue-light exposure, ROS colocalized with lipofuscin. FIG. 14B: A2E (MW=592) cannot pass 0.45 μm filters suggesting it forms aggregates. FIG. 14C: Correlation between MW and membrane cu-off for molecules that do not form aggregates. FIG. 14D: DIC and autofluorescence (green) reveals well defined A2E granules in cells co-stained with lysotracker (red). The yellow results from the green-red overlap. FIG. 14E: A2E crystal after solvent was evaporated on a cover-sleep. FIG. 14F: Galectin 3 puncta assay to evaluate lysosomes membrane damage. Both the positive control LLO and A2E induced Lysosome membrane permeabilization (LMP). FIG. 14G: Inactivation of cathepsin D in cells exposed to different doses of LLO. FIG. 14H: Cathepsin D in cells with different amounts of A2E. FIG. 14I: loss of Cathepsin D activity can be prevented with arimoclomol or necrostatin 7. FIG. 14J: Arimoclomol or Nec7 promote survival to A2E accumulation. FIG. 14K: The LMP inducer LLO promotes atypical necroptosis preventable with Nec7 and arimoclomol but not Nec1.

FIG. 15A: Heatmaps depicting protein levels, detected by mass spectrometry, in cells containing lipofuscin (15 μM-24 hrs A2E, loaded Overnight) vs healthy controls. Up- and down-regulated levels are represented with orange to blue scale, respectively. FIG. 15B: Causal networks association and hierarchical clustering analysis using Ingenuity Pathway (IPA) identified the cellular processes induced by lipofuscin. Dendrogram constructed based on Pearson correlation metric and average clustering method indicate that sub-lethal amounts of lipofuscin predominantly induce an anti-necroptotic response. The statistical significance, presented as the negative base-10 logarithm of the p-values obtained with IPA's right-tailed Fisher's exact test, is the probability that a cellular process or signaling pathway identified by IPA is not due to chance. −log(p-values are shown by the diameter of the circles. IPA also assigned z-scores that predicted the overall activation/inhibition state of the cellular/signaling pathways, indicated here as circle's colors (blue-orange scale). FIG. 15C: Identification with Ingenuity software of the main signaling cascades (eIF2a, eIF4, mTOR, ubiquitin proteasome system (UPS), integrin signaling (IS), and remodeling of epithelial adherence junctions (REAJ)) responsible for the inhibition of necroptotic cell death and survival in lipofuscin occupied cells. Circle size and color denote the statistical significance (−log(p-values)) and direction of the modulation, respectively. FIG. 15D: IPA analysis of the cellular processes individually controlled by eIF2a, eIF4, mTOR, ubiquitin proteasome system (UPS), integrin signaling (IS), and remodeling of epithelial adherence junctions (REAJ). FIG. 15E: Identification with IPA of the molecular processes through which eIF2a, eIF4, mTOR, and UPS counteract lipofuscin necro-toxicity.

FIG. 16A: Identity of the proteins modulated by lipofuscin and their association with the top anti-necroptotic signaling pathways, eIF2a, eIF4, mTOR, and UPS. The data indicate a profound reshape of the proteomics of the cell through changes in the initiation of protein translation and ubiquitination. FIG. 16B: Signs of increased catabolic machinery responsible for the degradation of proteins synthesized in the ER, induced by sublethal amounts of lipofuscin.

FIG. 17: IPA analysis of the signaling pathways induced by lipofuscin in vivo. Comparison, using Ingenuity Pathway Analysis of the differences in mRNA levels, detected by bulk RNAseq, between RPE/choroids from 100 days and 800 days of ABCA4−/−RDH8−/− double knockout (DKO) mice.

FIG. 18A: ARPE19 cells were pretreated for 1 hr with the agonists of: eIF2a (Salubrinal (SAL), SAL003, or Guanabenz); eIF4 (Briciclib or eFT508); mTOR (Rapamycin, Torin-1); or the unspecific protein translation inhibitor (Cyclohexamide CHX) and then incubated with lethal doses of A2E (25 μM) for an additional 24 hrs in the presence of these drugs. Viability was assessed with AlamarBlue®. Only SAL and SAL003, that targeted both cellular eIF2a phosphatases comprised of PP1 bound to either GADD34 or CreP, catalytic subunits, protected against lipofuscin. In contrast, Guanabenz, that only disrupts PP1-GADD34 association or eIF4 and mTOR activators, did not confer significant protection. FIG. 18B: Protection by SAL against increasing doses of A2E or all-trans retinal dimer (ATRD), two of the most abundant bisretinoids in the retinal lipofuscin. Viability was assessed with AlamarBlue®. FIG. 18C: SAL does not protect against the phototoxic decomposition of lipid bisretinoids. ARPE-19 cells were incubated O/N with a non-toxic amount of A2E (5 μM) to allow its incorporation into lysosomes and after changing the media for PBS and irradiating for 10 min with blue light cells were maintained for an additional 24 hrs in Optimem before evaluating viability, using AlamarBlue®. FIG. 18D: Fluorescence microscopy of cells incubated with lethal amounts of A2E (25 μM) in Optimem for 24 hrs. Green fluorescence corresponds to A2E deposits. Nuclei are stained with the DNA dye, Hoechst, in viable cells (blue) and with Hoechst and DRAQ7 (a DNA dye that only enter cells with disrupted membranes) in dead cells (purple).

FIG. 19A: Since SAL is a known activator of eIF2a through the preservation of its phosphorylated state and PERK is an eIF2a's kinase; 1 hr ARPE19 cells were pre-treated with SAL in the presence (or not) of a potent and specific PERK inhibitor (GSK2606414) followed by incubation with lethal doses of A2E (25 μM), in presence of these drugs, for an additional 24 hrs. Viability was assessed with AlamarBlue®. FIG. 19B: Knockdown of ATF4 did not prevent SAL from protecting against lipofuscin. Since ATF4 is a main downstream effector of PERK, ARPE-19 cells were transduced for 48 hrs with lentiviruses expressing scramble- or ATF4-shRNAs and then incubated with 25 μM A2E for additional 24 hrs, in the presence or not of SAL. Viability was assessed with AlamarBlue®.

FIG. 20A: Levels of spliced XBP1 (XBP1s), measured by quantitative real-time PCR as readout of IRE1α activity, increase in dose dependent fashion with the amount of lipofuscin accumulated in cells. IRE1α activity can be abrogated by treatment with SAL. IRE3, is a potent inhibitor of IRE1α, used here as control. FIG. 20B: Immunofluorescence staining of phospho-MLKL (green) showing that cells undergoing necroptosis, by A2E or ATRD, display phospho-MLKL membrane localization which can be abrogated by treatment with SAL. IRE3 was used as positive control of IRE1α inhibition.

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

Provided are methods and compositions for maintaining the viability of the RPE layer in subjects with Stargardt's disease (STGR1 and STGR3); vitelliform macular degeneration (Best's macular dystrophy or Best's disease); autosomal recessive cone-rod dystrophy (ar-CRD) and autosomal recessive retinitis pigmentosa (ar-RP), secondary to mutations in either the ABCA4 or RDH12 genes; and finally individuals with dry age-related macular degeneration (dry-AMD); choroidal melanoma; or severe ocular trauma associated with increased fundus autofluorescence (FAF) (C. J. Kennedy et al., Eye (Lond). 9 (Pt 6) (1995) 763-71; S. K. Verbakel et al., Prog. Retin. Eye Res. 66 (2018) 157-186; M. a van Driel et al., Ophthalmic Genet. 19 (1998) 117-122; A. Maugeri et al., Am. J. Hum. Genet. 67 (2000) 960-966; A. V. Cideciyan et al., Hum. Mol. Genet. 13 (2004) 525-534).

Excessive ER-stress in photoreceptors has been only associated with autosomal dominant forms of retinitis pigmentosa (adRP) but never with autosomal recessive retinitis pigmentosa (arRP). Comitato et al., Human Molecular Genetics, Vol. 25, No. 13 2801-2812 (2016). Chemical chaperones, i.e. drugs that increase the folding capacity in the cell and reduce the activity of all (IRE1α, PERK and ATF) UPR sensors were beneficial for adRP (S. X. Zhang et al., Exp. Eye Res. 125 (2014) 30-40; M. S. Gorbatyuk et al., Prog. Retin. Eye Res. (2020) 100860), but had no effect on ER-stress provoked by lipofuscin. In addition, treatment with Salubrinal, actually increased IRE1α activity in adRP retinas (Comitato et al., Human Molecular Genetics, Vol. 25, No. 13 2801-2812 (2016)), whereas the Examples herein demonstrate that Salubrinal reduced the same activity in cells with ER-stress due to lipofuscin.

In another model of retinal degeneration due to ER-stress induced by administration of oxidative stress causing agents, suppression of IRE1α resulted detrimental (S. X. Zhang et al., Exp. Eye Res. 125 (2014) 30-40; T. McLaughlin et al., Mol. Neurodegener. 13 (2018) 1-15). While KIRA inhibitors of IRE1α have been shown useful to protect photoreceptors, with massive amounts of misfolded proteins, from apoptosis (most cases of adRP) (R. Ghosh et al., Cell. (2014) 1-15; H. C. Feldman et al., ACS Chem. Biol. 11 (2016) 2195-2205) but were never used to protect RPE from lipid cytotoxicity.

The methods of the present disclosure are based on the following unexpected discoveries, that challenge current dogmas in the field of lipofuscin pathogenesis: 1) lipid-bisretinoids render the lysosomes in which they are trapped, leaky (increased lysosomal membrane permeabilization (LMP)); 2) cytosolic lipofuscin triggers the unfolded protein response (UPR); 3) lipofuscin elicited UPR induces via the ER-stress sensor IRE1α, the formation of an atypical necrosome that phosphorylates MLKL. Phospho-MLKL subsequently self-assembles into pores that damage the ER, lysosomal and plasma membranes, creating an amplification loop “ER-stress↔phospho-HLKL” that culminates with the necrosis of the lipofuscin occupied cells. The lipofuscin-elicited cell death pathway is fundamentally different from previously reported mechanisms of cell death because it does not involve oxidative stress, apoptosis or classical necrosomes containing RIPK1 and RIPK3 kinases.

The methods of the present disclosure preserve visual function of a subject suffering from lipofuscin pathologies such as Stargardt disease (STGD), autosomal recessive retinitis pigmentosa (RP), Age-Related Macular Degeneration (AMD), Best disease (BD), or autosomal recessive cone-rod dystrophy: i) by administering an effective amount of KIRA compounds (e.g., KIRA3, KIRA6, KIRA7, KIRA8); ii) by administering an effective amount of Salubrinal-derivatives; iii) by administering effective amounts of Necrostatin 7, Necrosulfonamide (NSA), Dabrafenib, or Arimoclomol which, inhibit the formation of phospho-MLKL, and so, interrupt the “phospho-MLKL→ER-stress→IRE1α→phospho-MLKL” loop. These strategies can be applied individually or in combination to halt the degenerative process.

The aforementioned approaches differ radically from all previous attempts used to date to protect the retina from lipofuscin cytotoxicity, including the blockage of lipid bisretinoids formation, the use of antiapoptotic agents, antioxidants, or light blocking lenses.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, or topically. Administration includes self-administration and the administration by another.

As used herein, the term “biological sample” means sample material derived from living cells. Biological samples may include tissues, cells, protein or membrane extracts of cells, and biological fluids (e.g., ascites fluid or cerebrospinal fluid (CSF)) isolated from a subject, as well as tissues, cells and fluids present within a subject. Biological samples of the present technology include, but are not limited to, samples taken from eye, breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, thymus, blood, hair, buccal, skin, serum, plasma, CSF, semen, prostate fluid, seminal fluid, urine, feces, sweat, saliva, sputum, mucus, bone marrow, lymph, and tears. Biological samples can also be obtained from biopsies of internal organs. Biological samples can be obtained from subjects for diagnosis or research or can be obtained from non-diseased individuals, as controls or for basic research. Samples may be obtained by standard methods including, e.g., venous puncture and surgical biopsy. In certain embodiments, the biological sample is a tissue sample obtained by needle biopsy.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the terms “individual”, “patient”, or “subject” can be an individual organism, a vertebrate, a mammal, or a human. In some embodiments, the individual, patient or subject is a human.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration. Pharmaceutically-acceptable carriers and their formulations are known to one skilled in the art and are described, for example, in Remington's Pharmaceutical Sciences (20th edition, ed. A. Gennaro, 2000, Lippincott, Williams & Wilkins, Philadelphia, Pa.). Examples of pharmaceutically-acceptable carriers include a liquid or solid filler, diluent, excipient, manufacturing aid (e.g., lubricant, talc magnesium, calcium or zinc stearate, or steric acid), or solvent encapsulating material, useful for introducing the active agent into the body.

As used herein, “prevention,” “prevent,” or “preventing” of a disorder or condition refers to one or more compounds that, in a statistical sample, reduces the occurrence of the disorder or condition in the treated sample relative to an untreated control sample, or delays the onset of one or more symptoms of the disorder or condition relative to the untreated control sample.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

Eye Diseases Associated with Retinal Cell Lipofuscin Cytotoxicity

Lipofuscin accumulates with age and can increase due to genetic predispositions and certain underlying conditions. See Molday R S, Zhong M, Quazi F, Biochim Biophys Acta 1791(7):573-83 (2009); Zaneveld J, et al. Genet Med 17(4):262-270 (2015); Allikmets R et al., Science 277(5333):1805-7 (1997); van Driel M a, Maugeri a, Klevering B J, Hoyng C B, Cremers F P, Ophthalmic Genet 19(3):117-122 (1998); Fishman G A, Ophthalmic Genet 31(4):183-9 (2010); Lim L S, Mitchell P, Seddon J M, Holz F G, Wong T Y, Lancet 379(9827):1728-1738 (2012); Swaroop A, Chew E Y, Rickman C B, Abecasis G R, Annu Rev Genomics Hum Genet 10:19-43 (2009); Charbel Issa P, Barnard A R, Herrmann P, Washington I, MacLaren RE (2015) Proc Natl Acad Sci 112(27):8415-20 (2017). ABCR mutations may occur in patients with age-related macular degeneration (AMD), Stargardt's disease, fundus flavimaculatus, cone dystrophy (COD, where only the cone cells undergo degeneration), and cone-rod dystrophy (CRD, where both rods and cones are undergo degeneration) and Retinitis pigmentosa. Examples of such ABCR mutations include, but are not limited to, ABCA4 D2177N, ABCA4 G1961E, ABCA4 G863A, ABCA4 1847delA, ABCA4 L541P, ABCA4 T2028I, ABCA4 N247I, ABCA4 E1122K, ABCA4 W499*, ABCA4 A1773V, ABCA4 H55R, ABCA4 A1038V, ABCA4 IVS30+1G→T, ABCA4 IVS40+5G→A, ABCA4 IVS14+1G→C, ABCA4 F1440del1cT, as well as those disclosed in Allikmets R et al., Science 277(5333):1805-7 (1997).

Retinitis pigmentosa (RP) is a group of diseases where photoreceptor cells die. RP is the most common inherited retinal dystrophy (IRD), with a worldwide prevalence of approximately 1:4000 (S. K. Verbakel, et al., Prog. Retin. Eye Res. 66, 157-186 (2018)). RP can be inherited in an autosomal dominant, autosomal recessive or X-linked manner. Over 40 genes have been associated with RP so far, with the majority of them expressed in either the photoreceptors or the retinal pigment epithelium. The tremendous heterogeneity of the disease makes the genetics of RP complicated. Ferrari et al., Current Genomics, 12, 238-249 (2011).

Typical fundus abnormalities include bone spicule pigmentation predominantly in the periphery and/or mid-periphery of the retina, which gives the name to the disease. The typical bone-spicule dark pigmentation, is observable with the ophthalmoscope and represents RPE cells that detached from the Bruch membrane following photoreceptor degeneration and migrated to intra-retinal perivascular sites, where they form melanin pigment deposits around the blood vessels. These bone spicules often arise in the mid-periphery, where the concentration of rod cells is the highest. Precisely what triggers RPE migration is unknown, but the migration is suspected to be facilitated by the reduced distance between the inner retinal vessels and the RPE, due to the degeneration of the photoreceptors. Almost all forms of RP go through a stage where no pigmentary changes exist in the retina. This stage may exist for decades before typical RP signs appear.

There are two autosomal recessive RP subtypes due to mutations in either the ABCA4 or RDH12 genes, which are very severe forms of RP with an early onset in life (R. F. Mullins, et al., Invest. Ophthalmol. Vis. Sci. 53, 1883-94 (2012); A. Schuster, et al., Investig. Ophthalmol. Vis. Sci. 48, 1824-1831 (2007)). A study in an Asian population revealed that they represent at least 3 and 2% of all RP cases, respectively (L. Huang, et al., Sci. Rep. 7, 1-10 (2017)). Examples of such RDH12 mutations include, but are not limited to, RDH12 p.G127X, RDH12 p.Q189X, RDH12 p.Y226C, RDH12 p.A269GfsX1, RDH12 p.L274P, RDH12 p.R65X, RDH12 p.H151D, RDH12 p.T155I, RDH12 p.V41L, RDH12 p.R314W and RDH12 p. V146D. Unlike autosomal dominant RP, autosomal recessive RP is characterized by high content of retinal lipofuscin in the RPE.

### Therapeutic Methods of the Present Technology

The present disclosure provides compositions that protect against lipofuscin cytotoxicity in retinal cells, e.g., arimoclomol, dabrafenib, necrosulfonamide (NSA), Necrostatin 7 (Nec7), KIRA compounds (e.g., KIRA3/6/7/8), Salubrinal, or SAL003, or pharmaceutically acceptable salts thereof.

In one aspect, the present disclosure provides a method for preventing or treating an eye disease associated with retinal cell lipofuscin-associated cytotoxicity in a subject in need thereof comprising administering to the subject an effective amount of at least one therapeutic agent selected from the group consisting of dabrafenib, necrosulfonamide (NSA), arimoclomol, a Kinase Inhibiting RNase Attenuator (KIRA) compound, salubrinal, SAL003 and any pharmaceutically acceptable salt thereof, wherein the eye disease associated with retinal cell lipofuscin-associated cytotoxicity is autosomal recessive retinitis pigmentosa (RP), Stargardt disease (STGD), Best disease (BD), cone-rod dystrophy, or ABCA4 mutant Age-Related Macular Degeneration (AMD). Examples of KIRA compounds include, but are not limited to, KIRA3, KIRA6, KIRA7, or KIRA8. In some embodiments, the subject comprises a mutation in ABCA4 and/or RDH12. The mutation in ABCA4 and/or RDH12 may be homozygous or heterozygous. Additionally or alternatively, in some embodiments of the methods disclosed herein, administration of the effective amount of the at least one therapeutic agent prevents exacerbation of lipofuscin-associated cytotoxicity in retinal cells in the subject.

In another aspect, the present disclosure provides a method for preventing or treating an ABCA4 mutant eye disease associated with retinal cell lipofuscin-associated cytotoxicity in a subject in need thereof comprising administering to the subject an effective amount of Necrostatin 7 (Nec7) or a pharmaceutically acceptable salt thereof, wherein the ABCA4 mutant eye disease associated with retinal cell lipofuscin-associated cytotoxicity is autosomal recessive retinitis pigmentosa (RP), cone-rod dystrophy, or Age-Related Macular Degeneration (AMD). In some embodiments, administration of the effective amount of Nec7 or pharmaceutically acceptable salt thereof prevents exacerbation of lipofuscin-associated cytotoxicity in retinal cells in the subject.

In any and all embodiments of the methods disclosed herein, the eye disease is genetic, non-genetic, or associated with aging. In some embodiments of the methods disclosed herein, the AMD is dry AMD. In other embodiments of the methods disclosed herein, the cone-rod dystrophy is autosomal recessive cone-rod dystrophy.

Additionally or alternatively, in some embodiments of the methods disclosed herein, the subject harbors at least one ABCA4 mutation selected from the group consisting of ABCA4 D2177N, ABCA4 G1961E, ABCA4 G863A, ABCA4 1847delA, ABCA4 L541P, ABCA4 T2028I, ABCA4 N247I, ABCA4 E1122K, ABCA4 W499*, ABCA4 A1773V, ABCA4 H55R, ABCA4 A1038V, ABCA4 IVS30+1G→T, ABCA4 IVS40+5G→A, ABCA4 IVS14+1G→C, and ABCA4 F1440del1cT. In any and all embodiments of the methods disclosed herein, the subject harbors at least one RDH12 mutation selected from the group consisting of RDH12 G127*, RDH12 Q189*, RDH12 Y226C, RDH12 A269Gfs*, RDH12 L274P, RDH12 R65*, RDH12 H151D, RDH12 T155I, RDH12 V41L, RDH12 R314W and RDH12 V146D. cone-rod dystrophy.

Additionally or alternatively, in some embodiments of the methods disclosed herein, the at least one therapeutic agent of the present technology (e.g., dabrafenib, NSA, arimoclomol, the KIRA compound, salubrinal, SAL003, Nec7, or the pharmaceutically acceptable salt thereof) reduces or eliminates lipofuscin bisretinoid (LB) lipid-induced phosphorylation and/or polymerization of MLKL. In any and all embodiments of the methods disclosed herein, the at least one therapeutic agent of the present technology (e.g., dabrafenib, NSA, arimoclomol, the KIRA compound, salubrinal, SAL003, Nec7, or the pharmaceutically acceptable salt thereof) reverses LB lipid-induced translocation of phosphorylated MLKL (pMLKL) to plasma membraned in retinal pigment epithelium cells. In certain embodiments, the LB lipids are selected from the group consisting of N-retinylidene-N-retinylethanolamine (A2E), an A2E isomer, an oxidized derivative of A2E, and all-trans-retinal dimers (ATRD).

In any of the preceding embodiments of the methods disclosed herein, the at least one therapeutic agent of the present technology (e.g., dabrafenib, NSA, arimoclomol, the KIRA compound, salubrinal, SAL003, Nec7, or the pharmaceutically acceptable salt thereof) reduces mRNA or protein levels of one or more genes associated with retinal degeneration, inflammation/angiogenesis, ER-stress and/or necroptosis. Examples of genes associated with retinal degeneration, inflammation/angiogenesis, ER-stress and/or necroptosis include, but are not limited to, EDN2, FGF2, GFAP, SERP, VEGF, CXCL15, XBP1s, SCAND1, CEBPA and HMGA. Additionally or alternatively, in some embodiments of the methods disclosed herein, the at least one therapeutic agent of the present technology (e.g., dabrafenib, NSA, arimoclomol, the KIRA compound, salubrinal, SAL003, Nec7, or the pharmaceutically acceptable salt thereof) inhibits or mitigates lipofuscin-induced necroptosis and/or reduces infiltration of activated microglia/macrophage in retinal pigment epithelium cells.

In any and all embodiments of the methods disclosed herein, the at least one therapeutic agent of the present technology (e.g., dabrafenib, NSA, arimoclomol, the KIRA compound, salubrinal, SAL003, Nec7, or the pharmaceutically acceptable salt thereof) is administered via topical, intravitreous, intraocular, subretinal, or subscleral administration. Additionally or alternatively, in some embodiments of the methods disclosed herein, the at least one therapeutic agent of the present technology (e.g., dabrafenib, NSA, arimoclomol, the KIRA compound, salubrinal, SAL003, Nec7, or the pharmaceutically acceptable salt thereof) is conjugated to an agent that targets retinal pigment epithelium cells. Examples of agents that target retinal pigment epithelium cells include, but are not limited to, tamoxifen, chloroquine (CQ)/hydroxychloroquine (HCQ), ethambutol (EMB), or sodium iodate (NaIO₃). Other examples of RPE targeting agents are described in Crisóstomo S, Vieira L, Cardigos J (2019) Retina:23-28; Michaelides M (2011) Arch Ophthalmol 129(1):30; Tsai R K, He M S, Chen Z Y, Wu W C, Wu W S (2011) Mol Vis 17(June):1564-1576; MacHalińska A, et al. (2010) Neurochem Res 35(11):1819-1827; Tsang S H, Sharma T (2018) Drug-Induced Retinal Toxicity. Atlas of Inherited Retinal Diseases, eds Tsang S H, Sharma T (Springer International Publishing, Cham), pp 227-232.

The term “pharmaceutically acceptable salt” means a salt prepared from a base or an acid which is acceptable for administration to a patient, such as a mammal (e.g., salts having acceptable mammalian safety for a given dosage regime). However, it is understood that the salts are not required to be pharmaceutically acceptable salts, such as salts of intermediate compounds that are not intended for administration to a patient. Pharmaceutically acceptable salts can be derived from pharmaceutically acceptable inorganic or organic bases and from pharmaceutically acceptable inorganic or organic acids. In addition, when one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) contain both a basic moiety, such as an amine, pyridine or imidazole, and an acidic moiety such as a carboxylic acid or tetrazole, zwitterions may be formed and are included within the term “salt” as used herein.

In one embodiment, the one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) may contain one or more basic functional groups, such as amino or alkylamino, and thereby, can form pharmaceutically-acceptable salts by reaction with a pharmaceutically-acceptable acid. These salts can be prepared in situ in the administration vehicle or the dosage form manufacturing process, or by separately reacting a purified compound of the present technology in its free base form with a suitable organic or inorganic acid, and isolating the salt thus formed during subsequent purification. In another embodiment, the one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) may contain one or more acidic functional groups, and thereby, can form pharmaceutically-acceptable salts by reaction with a pharmaceutically-acceptable base. These salts can likewise be prepared in situ in the administration vehicle or the dosage form manufacturing process, or by separately reacting the purified compound in its free acid form (e.g., hydroxyl or carboxyl) with a suitable base, and isolating the salt thus formed during subsequent purification.

Salts derived from pharmaceutically acceptable inorganic bases include ammonium, aluminum, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, ethylamine, diethylamine, 2-diethylaminoethanol, 2-dimethylaminoethanol, ethanolamine, diethanolamine, ethylenediamine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine, trimethylamine, tripropylamine, tromethamine and the like. Salts derived from pharmaceutically acceptable inorganic acids include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, 2-acetoxybenzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucuronic, mandelic, mucic, nicotinic, orotic, pamoic, pantothenic, sulfonic acids (e.g., benzenesulfonic, camphosulfonic, edisylic, ethanesulfonic, isethionic, methanesulfonic, naphthalenesulfonic, naphthalene-1,5-disulfonic, naphthalene-2,6-disulfonic and p-toluenesulfonic acids), xinafoic acid, valeric, oleic, palmitic, stearic, lauric, toluenesulfonic, methansulfonic, ethanedisulfonic, citric, ascorbic, maleic, oxalic, fumaric, phenylacetic, isothionic, succinic, tartaric, glutamic, salicylic, sulfanilic, napthylic, lactobionic, gluconic, laurylsulfonic acids, and the like.

Additionally or alternatively, in some embodiments, administration of the effective amount of the one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) or pharmaceutically acceptable salts thereof prevent exacerbation of lipofuscin-associated retinal cytotoxicity in the subject. In any and all embodiments of the methods disclosed herein, administration of the effective amount of the one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) or pharmaceutically acceptable salts thereof block, mitigate, or reverse lipofuscin associated cytotoxicity in retinal pigment epithelium cells.

In some embodiments of the methods disclosed herein, administration of the effective amount of the one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) or pharmaceutically acceptable salts thereof prevent, slow the onset, or lessen the severity of lipofuscin-associated damage or a disease or condition directly or indirectly associated with lipofuscin-associated damage in RPE cells of the subject. The subject can be of any gender (e.g., male or female), and/or can also be any age, such as elderly (generally, at least or above 60, 70, or 80 years of age), elderly-to-adult transition age subjects, adults, adult-to-pre-adult transition age subjects, and pre-adults, including adolescents (e.g., 13 and up to 16, 17, 18, or 19 years of age), children (generally, under 13 or before the onset of puberty), and infants. The subject can also be of any ethnic population or genotype. Some examples of human ethnic populations include Caucasians, Asians, Hispanics, Africans, African Americans, Native Americans, Semites, and Pacific Islanders.

Additionally or alternatively, in some embodiments, the one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) or pharmaceutically acceptable salts thereof are configured to localize to RPE cells.

Additionally or alternatively, in certain embodiments, the one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) or pharmaceutically acceptable salts thereof localize to RPE cells by being administered directly at, into, or in the adjacent vicinity of RPE cells, such as by injection or implantation.

In other embodiments, the one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) or pharmaceutically acceptable salts thereof localize to RPE cells by coupling the one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) or pharmaceutically acceptable salts thereof with a targeting agent that selectively targets RPE cells, and the one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) or pharmaceutically acceptable salts thereof may be administered at, into, or in the adjacent vicinity of RPE cells, or remotely from the RPE cells (e.g., by systemic administration). The cell-targeting agent (i.e., “targeting agent”) is any chemical entity that has the ability to bind to (i.e., “target”) a RPE cell. The cell-targeting agent may target any part of the RPE cell, e.g., cell membrane, organelle (e.g., lysosome or endosome), or cytoplasm. In one embodiment, the cell-targeting agent targets a component of a RPE cell in a selective manner. By selectively targeting a component of an RPE cell, the cell-targeting agent can, for example, selectively target certain components of cells over other types of cellular components. In other embodiments, the targeting agent targets cellular components non-selectively, e.g., by targeting cellular components found in most or all cells.

In various embodiments, the targeting agent can be, or include, for example, a peptide, dipeptide, tripeptide (e.g., glutathione), tetrapeptide, pentapeptide, hexapeptide, higher oligopeptide, protein, monosaccharide, disaccharide, trisaccharide, tetrasaccharide, higher oligosaccharide, polysaccharide (e.g., a carbohydrate), nucleobase, nucleoside (e.g., adenosine, cytidine, uridine, guanosine, thymidine, inosine, and S-Adenosyl methionine), nucleotide (i.e., mono-, di-, or tri-phosphate forms), dinucleotide, trinucleotide, tetranucleotide, higher oligonucleotide, nucleic acid, cofactor (e.g., TPP, FAD, NAD, coenzyme A, biotin, lipoamide, metal ions (e.g., Mg²⁺), metal-containing clusters (e.g., the iron-sulfur clusters), or a non-biological (i.e., synthetic) targeting group. Some particular types of proteins include enzymes, hormones, antibodies (e.g., monoclonal antibodies), lectins, and steroids.

Antibodies for use as targeting agents are generally specific for one or more cell surface antigens. In a particular embodiment, the antigen is a receptor. The antibody can be a whole antibody, or alternatively, a fragment of an antibody that retains the recognition portion (i.e., hypervariable region) of the antibody. Some examples of antibody fragments include Fab, Fc, and F(ab′)₂. In particular embodiments, particularly for the purpose of facilitating crosslinking of the antibody to the one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) or pharmaceutically acceptable salts thereof described herein, the antibody or antibody fragment can be chemically reduced to derivatize the antibody or antibody fragment with sulfhydryl groups. In certain embodiments, the targeting agent is a ligand of an internalized receptor of the target cell. For example, the targeting agent can be a targeting signal for acid hydrolase precursor proteins that transport various materials to lysosomes. One such targeting agent of particular interest is mannose-6-phosphate (M6P), which is recognized by mannose 6-phosphate receptor (MPR) proteins in the trans-Golgi. Endosomes are known to be involved in transporting M6P-labeled substances to lysosomes.

In other embodiments, the targeting agent is a peptide containing an RGD sequence, or variants thereof, that bind RGD receptors on the surface of many types of cells. Other targeting agents include, for example, transferrin, insulin, amylin, and the like. Receptor internalization may be used to facilitate intracellular delivery of the one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) or pharmaceutically acceptable salts thereof described herein. In certain embodiments, one cell-targeting molecule or group, or several (e.g., two, three, or more) of the same type of cell-targeting molecule or group are attached to the one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) or pharmaceutically acceptable salts thereof directly or via a linker. In other embodiments, two or more different types of targeting molecules are attached to the one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) or pharmaceutically acceptable salts thereof directly or via a linker.

Additionally or alternatively, in some embodiments, a fluorophore may be attached to the one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) or pharmaceutically acceptable salts thereof. Incorporation of one or more fluorophores can have several purposes. In some embodiments, one or more fluorophores are included in order to quantify cellular uptake and retention of the one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) or pharmaceutically acceptable salts thereof (e.g., by a fluorescence spectroscopic method).

As used herein, a “fluorophore” refers to any species with the ability to fluoresce (i.e., that possesses a fluorescent property). For example, in one embodiment, the fluorophore is an organic fluorophore. The organic fluorophore can be, for example, a charged (i.e., ionic) molecule (e.g., sulfonate or ammonium groups), uncharged (i.e., neutral) molecule, saturated molecule, unsaturated molecule, cyclic molecule, bicyclic molecule, tricyclic molecule, polycyclic molecule, acyclic molecule, aromatic molecule, and/or heterocyclic molecule (i.e., by being ring-substituted by one or more heteroatoms selected from, for example, nitrogen, oxygen and sulfur). In the particular case of unsaturated fluorophores, the fluorophore contains one, two, three, or more carbon-carbon and/or carbon-nitrogen double and/or triple bonds. In a particular embodiment, the fluorophore contains at least two (e.g., two, three, four, five, or more) conjugated double bonds aside from any aromatic group that may be in the fluorophore. In other embodiments, the fluorophore is a fused polycyclic aromatic hydrocarbon (PAH) containing at least two, three, four, five, or six rings (e.g., naphthalene, pyrene, anthracene, chrysene, triphenylene, tetracene, azulene, and phenanthrene) wherein the PAH can be optionally ring-substituted or derivatized by one, two, three or more heteroatoms or heteroatom-containing groups.

In other embodiments, the organic fluorophore is a xanthene derivative (e.g., fluorescein, rhodamine, Oregon green, eosin, and Texas Red), cyanine or its derivatives or subclasses (e.g., streptocyanines, hemicyanines, closed chain cyanines, phycocyanins, allophycocyanins, indocarbocyanines, oxacarbocyanines, thiacarbocyanines, merocyanins, and phthalocyanines), naphthalene derivatives (e.g., dansyl and prodan derivatives), coumarin and its derivatives, oxadiazole and its derivatives (e.g., pyridyloxazoles, nitrobenzoxadiazoles, and benzoxadiazoles), pyrene and its derivatives, oxazine and its derivatives (e.g., Nile Red, Nile Blue, and cresyl violet), acridine derivatives (e.g., proflavin, acridine orange, and acridine yellow), arylmethine derivatives (e.g., auramine, crystal violet, and malachite green), and tetrapyrrole derivatives (e.g., porphyrins and bilirubins). Some particular families of dyes considered herein are the Cy® family of dyes, the Alexa® family of dyes, the ATTO® family of dyes, and the Dy® family of dyes. The ATTO® dyes, in particular, can have several structural motifs, including, coumarin-based, rhodamine-based, carbopyronin-based, and oxazine-based structural motifs.

The fluorophore can be attached to the one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) or pharmaceutically acceptable salts thereof by any of the linking methodologies known in the art. For example, a commercial mono-reactive fluorophore (e.g., NHS-Cy5) or bis-reactive fluorophore (e.g., bis-NHS-Cy5 or bis-maleimide-Cy5) can be used to link the fluorophore to one or more molecules containing appropriate reactive groups (e.g., amino, thiol, hydroxy, aldehydic, or ketonic groups). Alternatively, the one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) or pharmaceutically acceptable salts thereof can be derivatized with one, two, or more such reactive groups, and these reactive portions reacted with a fluorophore containing appropriate reactive groups (e.g., an amino-containing fluorophore).

The one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) or pharmaceutically acceptable salts thereof can be administered by any route that permits contact with RPE cells. The administration can be, for example, ocular, parenteral (e.g., subcutaneous, intramuscular, or intravenous), topical, transdermal, intravitreous, retro-orbital, subretinal, subscleral, oral, sublingual, or buccal modes of administration. Some of the foregoing exemplary modes of administration can be achieved by injection. However, in some embodiments, injection is avoided by use of a slow-release implant in the vicinity of the retina (e.g., subscleral route) or by administering drops to the conjuctiva. The one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) or pharmaceutically acceptable salts thereof of the present technology may be administered locally, to the eyes of patients suffering from lipofuscin cytotoxicity including Stargardt, carriers of ABCA4 defective genes, dry AMD or at risk for developing retinal degeneration due to lipofuscin cytotoxicity. Local administration includes intravitreal, topical ocular, transdermal patch, subdermal, parenteral, intraocular, subconjunctival, or retrobulbar or subtenon's injection, trans-scleral (including iontophoresis), posterior juxtascleral delivery, or slow release biodegradable polymers or liposomes. The one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) or pharmaceutically acceptable salts thereof can also be delivered in ocular irrigating solutions. Concentrations may range from about 0.001 μM to about 100 μM, preferably about 0.01 μM to about 5 μM.

In some embodiments, the one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) or pharmaceutically acceptable salts thereof are administered, at least initially, at levels lower than that required in order to achieve a desired therapeutic effect, and the dose is gradually or suddenly increased until a desired effect is achieved. In other embodiments, the one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) or pharmaceutically acceptable salts thereof are administered, at least initially, at levels higher than that required in order to accelerate a desired therapeutic effect, and the dose gradually or suddenly moderated until a desired effect is achieved.

The selected dosage level will depend upon several factors, as determined by a medical practitioner. Some of these factors include the type of disease or condition being treated, the stage or severity of the condition or disease, the efficacy of the therapeutic compound being used and its bioavailability profile, as well as the specifics (e.g., genotype and phenotype) of the subject being treated, e.g., age, sex, weight, and overall condition.

Particularly for systemic modes of administration, the dosage can be, for example, in the range of about 0.01, 0.1, 0.5, 1, 5, or 10 mg per kg of body weight per day to about 20, 50, 100, 500, or 1000 mg per kilogram of body weight per day, or bi-daily, or twice, three, four, or more times a day. Particularly in embodiments where the one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) or pharmaceutically acceptable salts thereof are administered non-systemically directly at the retina, the dosage can disregard body weight, and can be in smaller amounts (e.g., 1-1000 μg per dose). In some embodiments, the daily dose of the one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) or pharmaceutically acceptable salts thereof is the lowest dose effective to produce a therapeutic effect. In some embodiments, the one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) or pharmaceutically acceptable salts thereof are not administered in discrete dosages, but in a continuous mode, such as provided by a slow release implant or intravenous line.

In one aspect, the present disclosure provides pharmaceutical compositions comprising arimoclomol, dabrafenib, necrosulfonamide (NSA), Necrostatin 7 (Nec7), KIRA compounds (e.g., KIRA3/6/7/8), Salubrinal, SAL003, and pharmaceutically acceptable salts thereof.

The one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) or pharmaceutically acceptable salts thereof may be formulated together with one or more pharmaceutically acceptable carriers (additives) and/or diluents, known in the art. The pharmaceutical compositions of the present technology may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) sublingually; (5) ocularly; (6) transdermally; or (7) nasally.

In some embodiments, pharmaceutical compositions of the present technology may contain one or more “pharmaceutically-acceptable carriers,” which as used herein, generally refers to a pharmaceutically-acceptable composition, such as a liquid or solid filler, diluent, excipient, manufacturing aid (e.g., lubricant, talc magnesium, calcium or zinc stearate, or steric acid), or solvent encapsulating material, useful for introducing the active agent into the body. Each carrier must be “acceptable” in the sense of being compatible with other ingredients of the formulation and not injurious to the patient. Examples of suitable aqueous and non-aqueous carriers that may be employed in the pharmaceutical compositions of the present technology include, for example, water, ethanol, polyols (such as glycerol, propylene glycol, polyethylene glycol, and the like), vegetable oils (such as olive oil), and injectable organic esters (such as ethyl oleate), and suitable mixtures thereof. Proper fluidity can be maintained, for example, by the use of coating materials, such as lecithin, by the maintenance of the required particle size in the case of dispersions, and by the use of surfactants.

In some embodiments, the formulations may include one or more of sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; alginic acid; buffering agents, such as magnesium hydroxide and aluminum hydroxide; pyrogen-free water; isotonic saline; Ringer's solution; ethyl alcohol; pH buffered solutions; polyesters, polycarbonates and/or polyanhydrides; preservatives; glidants; fillers; and other non-toxic compatible substances employed in pharmaceutical formulations.

Various auxiliary agents, such as wetting agents, emulsifiers, lubricants (e.g., sodium lauryl sulfate and magnesium stearate), coloring agents, release agents, coating agents, sweetening agents, flavoring agents, preservative agents, and antioxidants can also be included in the pharmaceutical composition. Some examples of pharmaceutically-acceptable antioxidants include: (1) water soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like. In some embodiments, the pharmaceutical formulation includes an excipient selected from, for example, celluloses, liposomes, micelle-forming agents (e.g., bile acids), and polymeric carriers, e.g., polyesters and polyanhydrides. Suspensions, in addition to the active compounds, may contain suspending agents, such as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof. Prevention of the action of microorganisms on the active compounds may be ensured by the inclusion of various antibacterial and antifungal agents, such as, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption, such as aluminum monostearate and gelatin.

Pharmaceutical formulations of the present technology may be prepared by any of the methods known in the pharmaceutical arts. The amount of active ingredient that can be combined with a carrier material to produce a single dosage form will vary depending upon the host being treated and the particular mode of administration. The amount of active ingredient that can be combined with a carrier material to produce a single dosage form will generally be that amount of the compound that produces a therapeutic effect. Generally, the amount of active compound will be in the range of about 0.1 to 99 percent, more typically, about 5 to 70 percent, and more typically, about 10 to 30 percent.

The compositions of the present technology may be administered locally, to the eyes of patients suffering from lipofuscin cytotoxicity including Stargardt, carriers of ABCA4 defective genes, dry AMD or at risk for developing retinal degeneration due to lipofuscin cytotoxicity. The one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) or pharmaceutically acceptable salts thereof can be incorporated into various types of ophthalmic formulations for delivery to the eye (e.g., topically, intracamerally, juxtasclerally, or via an implant). The one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) or pharmaceutically acceptable salts thereof may be combined with ophthalmologically acceptable preservatives, surfactants, viscosity enhancers, gelling agents, penetration enhancers, buffers, sodium chloride, and water to form aqueous, sterile ophthalmic suspensions or solutions or preformed gels or gels formed in situ.

In some embodiments, the compositions of the present technology (e.g., arimoclomol, dabrafenib, necrosulfonamide (NSA), Necrostatin 7 (Nec7), KIRA compounds (e.g., KIRA3/6/7/8), Salubrinal, SAL003, and pharmaceutically acceptable salts thereof) is administered 1-10 times a day, once a day, twice, three, four, or more times a day, 1-3 times a day, 2-4 times a day, 3-6 times a day, 4-8 times a day or 5-10 times a day. In some embodiments, the compositions of the present technology (e.g., arimoclomol, dabrafenib, necrosulfonamide (NSA), Necrostatin 7 (Nec7), KIRA compounds (e.g., KIRA3/6/7/8), Salubrinal, SAL003, and pharmaceutically acceptable salts thereof) is administered every day, every other day, 2-3 times a week, or 3-6 times a week.

In some embodiments, the dose of the compositions of the present technology (e.g., arimoclomol, dabrafenib, necrosulfonamide (NSA), Necrostatin 7 (Nec7), KIRA compounds (e.g., KIRA3/6/7/8), Salubrinal, SAL003, and pharmaceutically acceptable salts thereof) can be, for example, in the range of about 0.01, 0.1, 0.5, 1, 5, 10, or 100 mg per kg of body weight per day to about 20, 50, 100, 500, or 1000 mg per kilogram of body weight. Particularly in embodiments where the active substance is administered directly at the retina, the dosage administered can be independent of body weight, and can be in smaller amounts (e.g., 1-1000 μg per dose).

If dosed topically, the one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) or pharmaceutically acceptable salts thereof may be formulated as topical ophthalmic suspensions or solutions, with a pH of about 4 to 8. The one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) or pharmaceutically acceptable salts thereof will normally be contained in these formulations in an amount 0.001% to 5% by weight, or in an amount of 0.01% to 2% by weight. Thus, for topical presentation, 1 to 2 drops of these formulations would be delivered to the surface of the eye 1 to 4 times per day according to the discretion of a skilled clinician. In some embodiments, the pharmaceutical compositions of the present technology, containing therapeutically effective amounts of at least one composition of the present technology (e.g., arimoclomol, dabrafenib, necrosulfonamide (NSA), Necrostatin 7 (Nec7), KIRA compounds (e.g., KIRA3/6/7/8), Salubrinal, or SAL003, or pharmaceutically acceptable salts thereof), are delivered intravitreally either through an injection (perhaps microspheres), an intravitreal device, or placed in the sub-Tenon space by injection, gel, or implant, or by other methods discussed above. If delivered as a solution, the therapeutically effective amount of the one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) or pharmaceutically acceptable salts thereof in the composition might be about 18-44 μM, of a concentration of about 20-50%. If formulated as a suspension, a therapeutically effective amount of the one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) or pharmaceutically acceptable salts thereof is about 20-80%.

In another embodiment, the therapeutically effective amount of the one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) or pharmaceutically acceptable salts thereof is administered in the form of a mini-tablet, each weighing from about 1 mg to about 40 mg, or about 5 mg. From one to twenty such mini-tablets may be injected [dry] into the sub-Tenon space through a trochar in one dose, so that a total single dose of 50-100 mg [44-88 μM] is injected.

In some embodiments, the compositions of the present technology (e.g., arimoclomol, dabrafenib, necrosulfonamide (NSA), Necrostatin 7 (Nec7), KIRA compounds (e.g., KIRA3/6/7/8), Salubrinal, SAL003, and pharmaceutically acceptable salts thereof) is administered 1-10 times a day, once a day, twice, three, four, or more times a day, 1-3 times a day, 2-4 times a day, 3-6 times a day, 4-8 times a day or 5-10 times a day. In some embodiments, the compositions of the present technology (e.g., arimoclomol, dabrafenib, necrosulfonamide (NSA), Necrostatin 7 (Nec7), KIRA compounds (e.g., KIRA3/6/7/8), Salubrinal, SAL003, and pharmaceutically acceptable salts thereof) is administered every day, every other day, 2-3 times a week, or 3-6 times a week.

In some embodiments, the dose of the compositions of the present technology (e.g., arimoclomol, dabrafenib, necrosulfonamide (NSA), Necrostatin 7 (Nec7), KIRA compounds (e.g., KIRA3/6/7/8), Salubrinal, SAL003, and pharmaceutically acceptable salts thereof) can be, for example, in the range of about 0.01, 0.1, 0.5, 1, 5, 10, or 100 mg per kg of body weight per day to about 20, 50, 100, 500, or 1000 mg per kilogram of body weight. Particularly in embodiments where the active substance is administered directly at the retina, the dosage administered can be independent of body weight, and can be in smaller amounts (e.g., 1-1000 μg per dose).

Formulations of the present technology suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present technology as an active ingredient. The active compound may also be administered as a bolus, electuary, or paste.

Methods of preparing these formulations generally include the step of admixing a composition of the present technology or pharmaceutically acceptable salt thereof, with the carrier, and optionally, one or more auxiliary agents. In the case of a solid dosage form (e.g., capsules, tablets, pills, powders, granules, trouches, and the like), the active compound can be admixed with a finely divided solid carrier, and typically, shaped, such as by pelletizing, tableting, granulating, powderizing, or coating. Generally, the solid carrier may include, for example, sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and/or (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more auxiliary ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. The tablets, and other solid dosage forms of the active agent, such as capsules, pills and granules, may optionally be scored or prepared with coatings and shells, such as enteric coatings and other coatings well known in the pharmaceutical-formulating art. The dosage form may also be formulated so as to provide slow or controlled release of the active ingredient therein using, for example, hydroxypropylmethyl cellulose in varying proportions to provide the desired release profile, other polymer matrices, liposomes and/or microspheres. The dosage form may alternatively be formulated for rapid release, e.g., freeze-dried.

Generally, the dosage form is required to be sterile. For this purpose, the dosage form may be sterilized by, for example, filtration through a bacteria-retaining filter, or by incorporating sterilizing agents in the form of sterile solid compositions which can be dissolved in sterile water, or some other sterile injectable medium immediately before use. The pharmaceutical compositions may also contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms are typically a pharmaceutically acceptable emulsion, microemulsion, solution, suspension, syrup, or elixir of the active agent. In addition to the active ingredient, the liquid dosage form may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Dosage forms specifically intended for topical or transdermal administration can be in the form of, for example, a powder, spray, ointment, paste, cream, lotion, gel, solution, or patch. Ophthalmic formulations, such as eye ointments, powders, solutions, and the like, are also contemplated herein. The active compound may be mixed under sterile conditions with a pharmaceutically-acceptable carrier, and with any preservatives, buffers, or propellants that may be required. The topical or transdermal dosage form may contain, in addition to an active compound of this present technology, one or more excipients, such as those selected from animal and vegetable fats, oils, waxes, paraffins, starch, tragacanth, cellulose derivatives, polyethylene glycols, silicones, bentonites, silicic acid, talc and zinc oxide, and mixtures thereof. Sprays may also contain customary propellants, such as chlorofluorohydrocarbons and volatile unsubstituted hydrocarbons, such as butane and propane.

For purposes of this present technology, transdermal patches may provide the advantage of permitting controlled delivery of a compound of the present technology into the body. Such dosage forms can be made by dissolving or dispersing the compound in a suitable medium. Absorption enhancers can also be included to increase the flux of the compound across the skin. The rate of such flux can be controlled by either providing a rate-controlling membrane or dispersing the compound in a polymer matrix or gel.

Pharmaceutical compositions of this present technology suitable for parenteral administration generally include one or more compounds of the present technology in combination with one or more pharmaceutically-acceptable sterile isotonic aqueous or nonaqueous solutions, dispersions, suspensions or emulsions, or sterile powders that may be reconstituted into sterile injectable solutions or dispersions prior to use, which may contain sugars, alcohols, antioxidants, buffers, bacteriostats, or solutes that render the formulation isotonic with the blood of the intended recipient.

In some cases, in order to prolong the effect of a drug, it may be desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms can be made by forming microencapsule matrices of the active compound in a biodegradable polymer, such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations can also be prepared by entrapping the drug in liposomes or microemulsions that are compatible with body tissue.

The pharmaceutical composition may also be in the form of a microemulsion. In the form of a microemulsion, bioavailability of the active agent may be improved. Reference is made to Dordunoo, S. K., et al., Drug Development and Industrial Pharmacy, 17(12), 1685-1713, 1991, and Sheen, P. C., et al., J. Pharm. Sci., 80(7), 712-714, 1991, the contents of which are herein incorporated by reference in their entirety.

The pharmaceutical composition may also contain micelles formed from a compound of the present technology and at least one amphiphilic carrier, in which the micelles have an average diameter of less than about 100 nm. In some embodiments, the micelles have an average diameter less than about 50 nm, or an average diameter less than about 30 nm, or an average diameter less than about 20 nm.

While any suitable amphiphilic carrier is considered herein, the amphiphilic carrier is generally one that has been granted Generally-Recognized-as-Safe (GRAS) status, and that can both solubilize the compound of the present technology and microemulsify it at a later stage when the solution comes into a contact with a complex water phase (such as one found in the living biological tissue). Usually, amphiphilic ingredients that satisfy these requirements have HLB (hydrophilic to lipophilic balance) values of 2-20, and their structures contain straight chain aliphatic radicals in the range of C-6 to C-20. Some examples of amphiphilic agents include polyethylene-glycolized fatty glycerides and polyethylene glycols.

Some amphiphilic carriers are saturated and monounsaturated polyethyleneglycolyzed fatty acid glycerides, such as those obtained from fully or partially hydrogenated various vegetable oils. Such oils may advantageously consist of tri-. di- and mono-fatty acid glycerides and di- and mono-polyethyleneglycol esters of the corresponding fatty acids, such as a fatty acid composition including capric acid 4-10, capric acid 3-9, lauric acid 40-50, myristic acid 14-24, palmitic acid 4-14 and stearic acid 5-15%. Another useful class of amphiphilic carriers includes partially esterified sorbitan and/or sorbitol, with saturated or mono-unsaturated fatty acids (SPAN-series) or corresponding ethoxylated analogs (TWEEN-series). Commercially available amphiphilic carriers are particularly contemplated, including the Gelucire®-series, Labrafil®, Labrasol®, or Lauroglycol®, PEG-mono-oleate, PEG-di-oleate, PEG-mono-laurate and di-laurate, Lecithin, Polysorbate 80.

Hydrophilic polymers suitable for use in the pharmaceutical composition are generally those that are readily water-soluble, can be covalently attached to a vesicle-forming lipid, and that are tolerated in vivo without substantial toxic effects (i.e., are biocompatible). Suitable polymers include, for example, polyethylene glycol (PEG), polylactic (also termed polylactide), polyglycolic acid (also termed polyglycolide), a polylactic-polyglycolic acid copolymer, and polyvinyl alcohol. Exemplary polymers are those having a molecular weight of from about 100 or 120 daltons up to about 5,000 or 10,000 daltons, and more preferably from about 300 daltons to about 5,000 daltons. In certain embodiments, the polymer is polyethylene glycol having a molecular weight of from about 100 to about 5,000 daltons, or a molecular weight of from about 300 to about 5,000 daltons, or a molecular weight of 750 daltons, i.e., PEG(750). Polymers may also be defined by the number of monomers therein. In some embodiments, the pharmaceutical compositions of the present technology utilize polymers of at least about three monomers, such PEG polymers comprising of at least three monomers, or approximately 150 daltons. Other hydrophilic polymers that may be suitable for use in the present technology include polyvinylpyrrolidone, polymethoxazoline, polyethyloxazoline, polyhydroxypropyl methacrylamide, polymethacrylamide, polydimethylacrylamide, and derivatized celluloses such as hydroxymethylcellulose or hydroxyethylcellulose.

In certain embodiments, the pharmaceutical composition includes a biocompatible polymer selected from polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, and copolymers thereof.

The pharmaceutical composition may also be in liposomal form. Liposomes contain at least one lipid bilayer membrane enclosing an aqueous internal compartment. Liposomes may be characterized by membrane type and by size. Small unilamellar vesicles (SUVs) have a single membrane and typically range from 0.02 to 0.05 μm in diameter; large unilamellar vesicles (LUVS) are typically larger than 0.05 μm Oligolamellar large vesicles and multilamellar vesicles have multiple, usually concentric, membrane layers, and are typically larger than 0.1 μm. The liposomes may also contain several smaller vesicles contained within a larger vesicle, i.e., multivesicular vesicles.

In some embodiments, the pharmaceutical composition includes liposomes containing one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) or pharmaceutically acceptable salts, where the liposome membrane is formulated to provide an increased carrying capacity. Alternatively or additionally, the one or more compositions of the present technology (e.g., arimoclomol, dabrafenib, necrosulfonamide (NSA), Necrostatin 7 (Nec7), KIRA compounds (e.g., KIRA3/6/7/8), Salubrinal, or SAL003) or pharmaceutically acceptable salts may be contained within, or adsorbed onto, the liposome bilayer of the liposome. In some embodiments, the active agent may be aggregated with a lipid surfactant and carried within the liposome's internal space. In such cases, the liposome membrane is formulated to resist the disruptive effects of the active agent-surfactant aggregate. In certain embodiments, the lipid bilayer of a liposome contains lipids derivatized with polyethylene glycol (PEG), such that the PEG chains extend from the inner surface of the lipid bilayer into the interior space encapsulated by the liposome, and extend from the exterior of the lipid bilayer into the surrounding environment.

Active agents contained within liposomes are preferably in solubilized form. Aggregates of surfactant and active agent (such as emulsions or micelles containing the active agent of interest) may be entrapped within the interior space of liposomes. A surfactant typically serves to disperse and solubilize the active agent. The surfactant may be selected from any suitable aliphatic, cycloaliphatic or aromatic surfactant, including but not limited to biocompatible lysophosphatidylcholines (LPCs) of varying chain lengths, e.g., from about 14 to 20 carbons. Polymer-derivatized lipids, such as PEG-lipids, may also be utilized for micelle formation as they will act to inhibit micelle/membrane fusion, and as the addition of a polymer to surfactant molecules decreases the critical micelle concentration (CMC) of the surfactant and aids in micelle formation. Preferred are surfactants with CMCs in the micromolar range; higher CMC surfactants may be utilized to prepare micelles entrapped within liposomes of the present technology, however, micelle surfactant monomers could affect liposome bilayer stability and would be a factor in designing a liposome of a desired stability.

Liposomes according to the present technology may be prepared by any of a variety of techniques known in the art, such as described in, for example, U.S. Pat. No. 4,235,871 and International Published Application WO 96/14057, the contents of which are incorporated herein by reference in their entirety. For example, liposomes may be prepared by diffusing a lipid derivatized with a hydrophilic polymer into preformed liposomes, such as by exposing preformed liposomes to micelles composed of lipid-grafted polymers, at lipid concentrations corresponding to the final mole percent of derivatized lipid which is desired in the liposome. Liposomes containing a hydrophilic polymer can also be formed by homogenization, lipid-field hydration, or extrusion techniques, as are known in the art. By another methodology, the active agent is first dispersed by sonication in a lysophosphatidylcholine or other low critical micelle concentration (CMC) surfactant (including polymer grafted lipids) that readily solubilizes hydrophobic molecules. The resulting micellar suspension of active agent is then used to rehydrate a dried lipid sample that contains a suitable mole percent of polymer-grafted lipid, or cholesterol. The lipid and active agent suspension is then formed into liposomes using extrusion techniques well known in the art, and the resulting liposomes separated from the unencapsulated solution by standard column separation.

In some embodiments, the liposomes are prepared to have substantially homogeneous sizes in a selected size range. One effective sizing method involves extruding an aqueous suspension of the liposomes through a series of polycarbonate membranes having a selected uniform pore size. The pore size of the membrane will correspond roughly with the largest sizes of liposomes produced by extrusion through the membrane (U.S. Pat. No. 4,737,323, the contents of which are herein incorporated by reference in their entirety).

The release characteristics of a formulation of the present technology depend on several factors, including, for example, the type and thickness of the encapsulating material, the concentration of encapsulated drug, and the presence of release modifiers. If desired, the release can be manipulated to be pH dependent, such as by using a pH-sensitive coating that releases only at a low pH, as in the stomach, or releases at a higher pH, as in the intestine. An enteric coating can be used to prevent release from occurring until after passage through the stomach. Multiple coatings or mixtures of cyanamide encapsulated in different materials can be used to obtain an initial release in the stomach, followed by later release in the intestine. Release can also be manipulated by inclusion of salts or pore-forming agents, which can increase water uptake or release of drug by diffusion from the capsule. Excipients that modify the solubility of the drug can also be used to control the release rate. Agents that enhance degradation of the matrix or release from the matrix can also be incorporated. The agents can be added to the drug, added as a separate phase (i.e., as particulates), or can be co-dissolved in the polymer phase depending on the compound. In all cases, the amount is preferably between 0.1 and thirty percent (w/w polymer). Some types of degradation enhancers include inorganic salts, such as ammonium sulfate and ammonium chloride; organic acids, such as citric acid, benzoic acid, and ascorbic acid; inorganic bases, such as sodium carbonate, potassium carbonate, calcium carbonate, zinc carbonate, and zinc hydroxide; organic bases, such as protamine sulfate, spermine, choline, ethanolamine, diethanolamine, and triethanolamine; and surfactants, such as a Tween™ or Pluronic™ commercial surfactant. Pore-forming agents that add microstructure to the matrices (i.e., water-soluble compounds, such as inorganic salts and sugars) are generally included as particulates.

Uptake can also be manipulated by altering residence time of the particles in the body. This can be achieved by, for example, coating the particle with, or selecting as the encapsulating material, a mucosal adhesive polymer. Examples include most polymers with free carboxyl groups, such as chitosan, celluloses, and especially polyacrylates (as used herein, polyacrylates refers to polymers including acrylate groups and modified acrylate groups such as cyanoacrylates and methacrylates).

### EXAMPLES

The present technology is further illustrated by the following Examples, which should not be construed as limiting in any way.

### Example 1: Materials and Methods

Cell culture. Human retinal pigment epithelium (ARPE-19) cells were obtained from ATCC and were cultured in Dulbecco's modified Eagle's medium (DMEM) supplemented with 10% FBS and 1% penicillin/streptomycin. Cells were kept in an incubator with 5% C02 and 95% humidified air at 37° C. Human fetal RPE (hfRPE) cells from donors at 16 to 18 weeks gestation were cultured at 37° C., 5% C02 in RPE medium. To grow polarized hfRPE cells, the cells were seeded in 12 well transwell plate in 1% RPE medium with Rock kinase inhibitor for the first week. After the first week, cells were cultured in normal 1% RPE medium for 3 weeks. Cells were used in passage 1.

Blue light treatment. ARPE19 cells were grown at 70% confluence and were treated with or without 20 μM A2E in culture medium for 24 h. Then, HBSS replaced the cell culture medium before blue light treatment. ARPE19 cells with or without A2E intake, were illuminated by 460+20 nm wavelength light for 20 min.

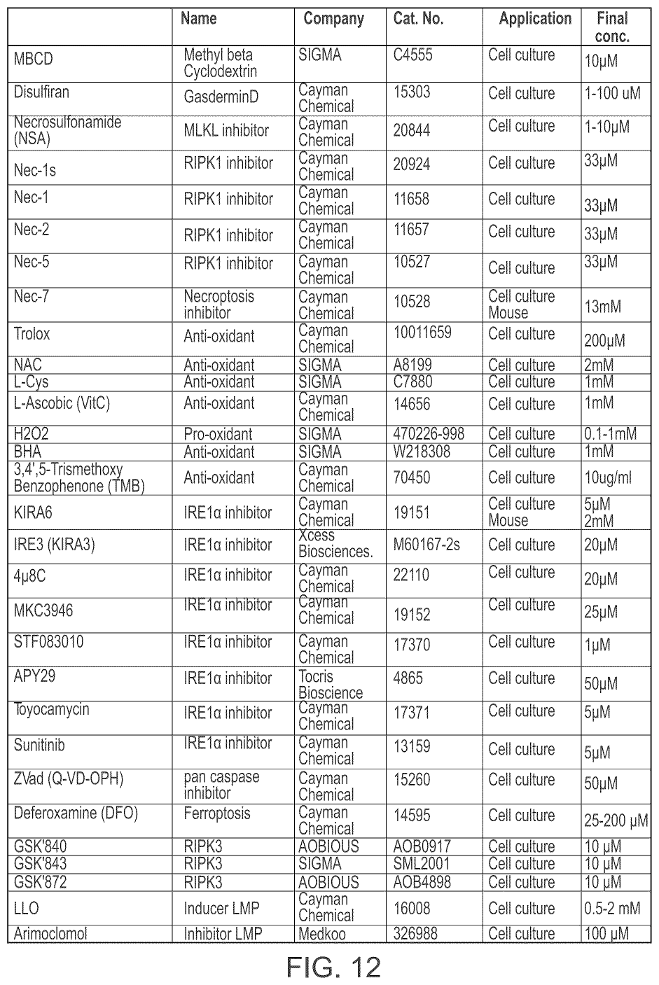

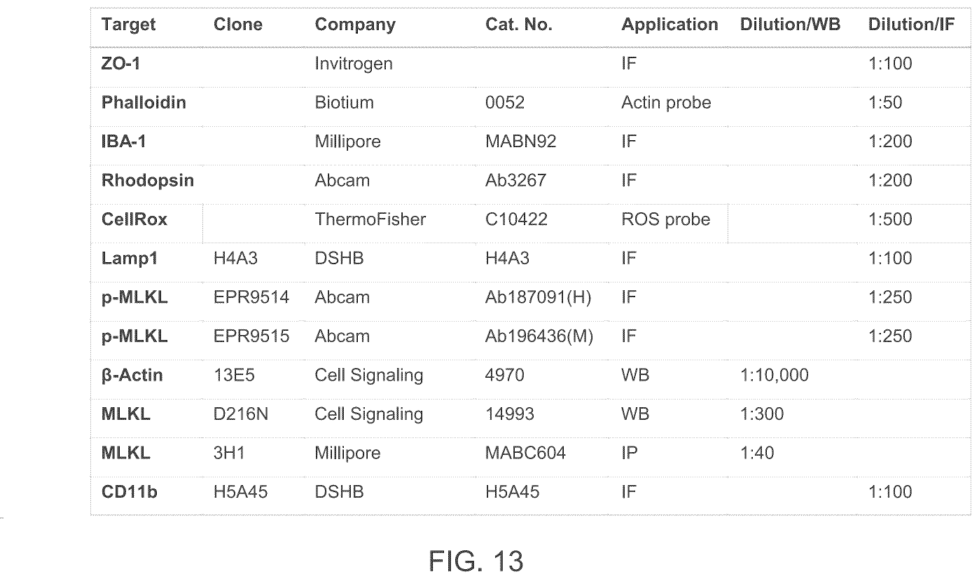

Cell Viability Assay. ARPE19 cells (80% confluent) pre-treated with inhibitors (FIG. 12) or not, received A2E/ATRD or vehicle (control) and were incubated in serum free OptiMEM medium for 24 h at 37° C. Viability was assessed at 24 h with AlamarBlue® (Sigma, cat #199303), or microscopy with NUC405/DRAQ7. 1× AlamarBlue solution prepared in OptiMEM was used to replace the media supernatant and the plate's fluorescence was determined at 555 nm excitation/585 nm emission with a SpectraMax M (Molecular Devices, CA, USA), after 1 h. For real time monitoring of cell-death by automated fluorescence microscopy, final concentration of 2 μM NUCView caspase-3 substrate 405 (Biotium, cat.no.10407) and 0.6 μM of DRAQ7 (Invitrogen, cat #D15106) were added per well and the fluorescence signals were monitored every 30 min according to manufacturers' instructions. The exact timing of appearance of far-red (DRAQ7=plasma membrane leakage) and blue (NUC405=caspase 3 activation) fluorescence signals was critical to differentiate apoptosis and necrosis from secondary events, such as membrane damage and generalized proteolytic activation during the late phase of apoptosis and necrosis, respectively. For blue light treatment, A2E/ATRD or vehicle loaded cells' media was replaced with HBSS and cells were exposed for 15 min to a 90-Watt high power LED light (cat #2506BU) with 430+20 nm wavelength illumination and HBSS was replaced by OptiMEM medium at zero time of treatment.

Real time monitoring of necrotic and apoptotic cell-death. Cell-death assay to study Lipofuscin's dark toxicity was performed. 90% confluent ARPE-19 or hfRPE were incubated overnight in serum free media supplemented with indicated LB concentration. Viability was assessed at 24 hrs by AlamarBlue®, or microscopy with NUC405/DRAQ7. Real time monitoring of necrotic (red) and apoptotic (blue) cell-death was performed by automated fluorescence microscopy. The exact timing of appearance of far-red (DRAQ7=plasma membrane leakage) and blue (NUC405=caspase 3 activation) fluorescence signals was critical to differentiate apoptosis and necrosis from secondary events, such as membrane damage and generalized proteolytic activation during the late phase of apoptosis and necrosis, respectively.

Chemical inhibitors. Chemical inhibitors used in this study are described in FIG. 12. ARPE19 cells were treated with A2E/ATRD in a 24 h in a 48 well plate with or without the tested inhibitors.

Kinase activity assay. ARPE19 cells were treated with A2E for 6 h followed by total cell lysates preparation by syringe (20 times) with the use of lysis buffer (20 mM TrisHCl, pH 7.5, 150 mM NaCl, 1% Triton, protease-phosphatase inhibitor 1×). Supernatant was collected after centrifugation at 15000×g for 10 mins. 200 μl (3 mg/ml) of protein were taken and incubated overnight with primary RIP3 (13526,CS) or MLKL antibody at 4° C. with rotation. Next, 20 μl agarose beads A (9863,CS) were added and kept at 4° C. with rotation for 3 h followed by centrifugation at 15000×g for 30 sec. Pellet were washed and dissolved in 20 μl of kinase buffer (40 mM TrisHCl pH7.4, 20 mM MgCl₂, 0.1 mg/ml BSA) with substrate and ATP (1 mM) and incubated at 30° C. for 1 h (700 speed) for kinase assay. At the end of the incubation 10 μl of the sample was taken in 384 well plate and 5 μl ADP glo solution was added (V6930, Promega) and kept at room temperature for 40 min in the dark followed by addition of 10 μl kinase detection reagent. After 20 min at room temperature, luminometer readings were taken.

Animals. Pigmented ABCA4^{-/-} RDH8^{-/-} double knock-out mice (DKO), free of rd8 mutation, were purchased from Jackson laboratories and every 10 generations were in house backcrossed to the C57BL6J control strain, to prevent genetic drifts. Genotyping was performed at Transnetyx (Memphis, TN). Only mice with ABCA4, RDH8, RPE65-Leu450 but no crb1 mutations (retinal degeneration slow) were maintained in the colony. Controls C57BL/6J (Rpe65-Leu450, crb1negative) were also Jackson's lab. All mice were housed at Weill Cornell Medicine's animal facility under a 12 h light (˜10 lux)/12 h dark cycle environment or under complete darkness. Experimental manipulations in the dark were done under dim red light transmitted through a Kodak No. 1 safelight filter (transmittance >560 nm). No retinal degeneration or necroptosis markers were appreciably detected in 20 months or older C57BL6/N (Rpe65-Met450, crb1 positive) obtained from the NAI/NIH. All animal procedures and experiments were approved by the Animal Care and Use Committee of Weill Cornell Medical College in agreement with the guidelines established by the NIH Office of Laboratory Animal Welfare and the Association of Research for Vision and Ophthalmology (ARVO) statement for the use of animals in ophthalmic research.

Tissue preparation. Mice were euthanized with C02 and mouse eyes were immediately enucleated. For H&E staining, mouse eyes were immersed in 4% paraformaldehyde (PFA), 16.8% isopropyl alcohol, 2% trichloroacetic acid and 2% ZnCl₂ in phosphate buffer directly and sent for paraffin embedding and sectioning. For lipofuscin images, mouse eyes were immersed into 4% PFA for one hour before dissection. RPE layer was dissected out from mouse eyes and carefully flat-mounted on slides for lipofuscin assessment under fluorescence microscope. Immunofluorescence images were taken using Zeiss Spinning Disk Confocal Microscope (Zeiss, Jena, Germany).

RPE and Neural retina flat mounts. Mouse eyes were enucleated and placed in 4% PFA in PBS for 1 h at room temperature. After fixing, a 23G needle was used to make a hole at the limbus area and iris scissors used to cut around the circumference of the limbus, remove the cornea, iris and lens, separate the neuronal retina and sclera by micro-forceps. Neuronal retina and RPE layer were dissected out and incubated with blocking buffer (1% BSA and 0.3% Triton-X-100 in PBS) for at least 20 min. Primary antibodies were added to the blocking buffer and kept at 4° C. overnight. Retina and RPE layer were washed and incubated with secondary antibodies for at least 30 min at room temperature, then washed with PBS three times. Under the dissection microscope, retina or RPE layer was cut into a four-leaf clover shape and mount on the slides in mount medium (EMS glycerol mounting medium with DAPI and DABCO, cat. No. 17989-61). Slides were stored at 4° C. until imaging.

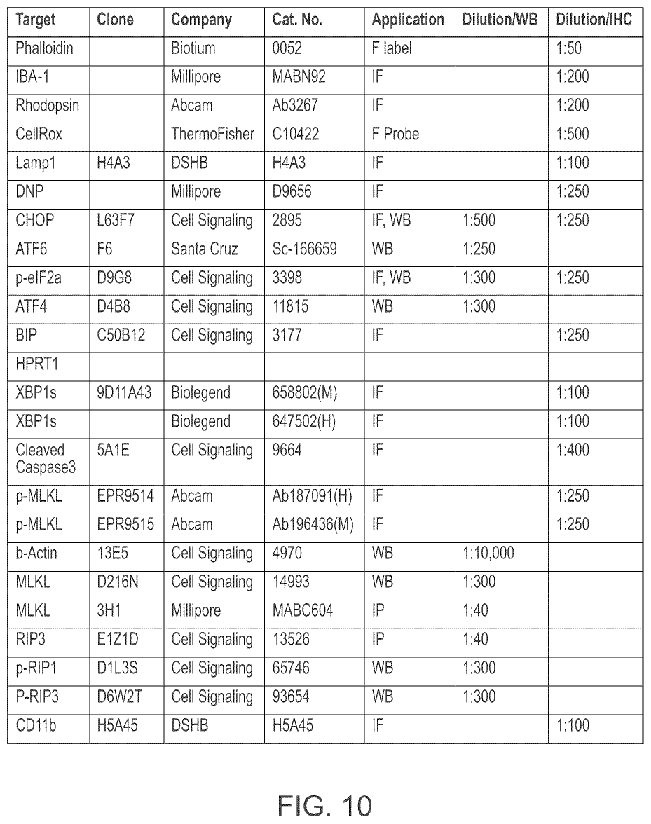

Cryosections and Immunofluorescence staining. Mouse eyes were fixed with 4% PFA and penetrated with 30% sucrose overnight at 4° C., then cryopreserved in Tissue-Tek OCT compound (Sakura Finetek, Torrance, CA, USA) and cut into 15 μm sections. Sections were blocked with 1% BSA and 0.1% Triton-X-100 PBS, and then immunofluorescence staining was performed using standard methods and the appropriate dilutions of primary antibodies against p- p-MLKL (cell signaling), XBP1s (Biogend), Iba-1 (Abcam), CD11b (Millipore), Lamp2 (hybridoma bank), CellRox and DRAQ7 (Invitrogen), Rhodopsin (Abcam), phalloidin-CF660 (Biotium, cat.no. 0052), Hoechst33324, caspase3 (clone9664, Cell signaling). Subsequently, slides were incubated with Alexa-647, Alexa-594 secondary antibodies, and counterstained with Hoechst33324. Negative controls were included in each staining, and slides were mounted with anti-fade mount medium. Slides were stored at 4° C. before analysis on SD microscope using Zen software. For paraffin sections, mouse eyes were embedded in paraffin and cut into 5-μm sections. After deparaffinzation using standard protocol, slides were incubated with blocking buffer for 2 hrs and then primary antibodies overnight. All antibodies used for immunofluorescence staining were listed in FIGS. 10 & 13.

Demelanization. Paraffin slides were removed of paraffin using a standard protocol, as described herein. Slides were washed 4 times in Xylene 4 min/wash, 20 dips in 100% Ethanol and repeat 4 times, then air dried the slides for 10 min. For melanin bleach, slides were immersed into PBS with 10% H₂O₂ at 55° C. for 5 min or until melanin is bleached. Slides were blocked with blocking buffer and performed immunofluorescence staining as above mentioned.

H&E. H&E staining was performed using standard protocol, as described herein. Slides were removed of paraffin using protocol as above described. Slides were air dried. Slides on the rack were put into Xylene for 2 min (repeated once); then in 100% ethanol for 2 min (repeated once); and then 95% ethanol for 2 min once. Slides then were put in Hematoxylin for 3 min, followed by Eosin for 45 seconds, 95% ethanol for 1 min, 100% ethanol for 1 min twice, then in mounting medium and were ultimately coverslipped.

Pharmacological treatment of mice. KIRA6 (Cayman Chemical, item no. 19151) was injected intravitreously with 1 μL total volume. KIRA6 concentration is 20 μg/ml. The control eye received an equal amount of mock reagent (DMSO). Mouse was weighed and anesthetized with Ketamine cocktail at 10 mg/kg, then mouse eyes were dilated with Tropicamide. The exact volume of Mock reagent or Kira6 was determined by 10 μl Hamilton syringe. Under surgery microscope, mouse eye was placed in the center of the field, 34 gauge of needle was inserted into mouse eye at the Ora serrata and towards ONH. Once the needle was inside the mouse eye, 2 μl of the contents in the Hamilton syringe was injected. The needle was slowly withdrawn to prevent the reflux of the solution. Nec7 (2 μL of 33 mM stock in DMSO) was intravitreally injected in the left eye and an equal amount of vehicle (DMSO) was administered to the right eye. After intravitreal injection, the mouse was placed in a warm place until it completely woke up. Single intravitreal injection of Nec7 decreased pMLKL staining with the respective companion eye was performed. Immunofluorescence staining with anti-p-MLKL (red) and XBP1s (green) and LB (white) of RPE flat mount samples from 700 day old mice is shown (n=3). Bar=20 μm.

Lipofuscin synthesis. A2E was synthesized and purified by HPLC (>97%) according to a published protocol. Quality of the material was assessed by mass-spect and UV absorbance between 250 and 600 nm.

HPLC analysis of bisretinoids content. Bisretinoids were extracted from mouse eyecups under red dim light. Briefly, single mouse eyecup (containing RPE/choroid/sclera, devoid of neural retina) or ARPE-19 cells were washed with phosphate buffer (PBS) and homogenized in 1 mL PBS. Four milliliters chloroform/methanol (2:1, vol/vol) was added, and the samples were extracted with the addition of 4 mL chloroform and 3 mL dH₂O, followed by centrifugation at 1000×g for 10 min. Extraction was repeated with the addition of 4 mL chloroform. Organic phases were pooled, filtered, dried under a stream of argon, and redissolved in 100 μL 2-propanol. Bisretinoid extracts were analyzed by normal-phase HPLC with a silica column (Zorbax-Sil 5 μm, 250×4.6 mm; Agilent Technologies, Wilmington, DE) as previously described (Sparrow J R, et al. (2003) J Biol Chem 278(20):18207-13). The mobile phase was hexane/2-propanol/ethanol/25 mM potassium phosphate/glacial acetic acid (485:376:100:45:0.275 vol/vol) that was filtered before use. The flow rate was 1 mL/min. Column and solvent temperatures were maintained at 40° C. Absorption units at 435 nm were converted to picomoles using a calibration curve with an authentic A2E standard and the published molar extinction coefficient for A2E; the identity of each bisretinoid peak was confirmed by online spectral analysis.

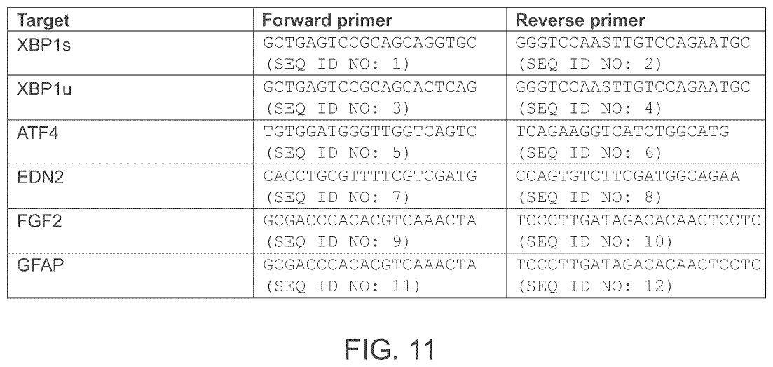

RNA isolation and quantitative PCR. Total RNA was extracted from cultured cells or mouse eye RPE layer using the RNeasy Mini kit (QIAGEN). The total RNA was digested with deoxyribonuclease I to prevent amplification of genomic DNA. The total RNA then was reversed transcribed using High-Capacity cDNA Reverse Transcription Kit (ThermoFisher Scientific, cat.no. 4368814) and analyzed gene expression using SYBR Green Master Mix (ThermoFisher Scientific, cat.no. 4472908) in an Applied Biosystems StepOne real time PCR machine. GAPDH was used as a reference gene. Primer sequences are displayed in FIG. 11. After genes were amplified by real time PCR, some of the PCR products were separated by 2.5% agarose gel.

RNAseq. Cultured ARPE19 cells were treated with or without 15 uM A2E for 24 hours, then cells were harvested and total RNA extracted. Proteins were prepared for mass spectrometry analysis. RNAseq profiles were analyzed further with Ingenuity Pathway Analysis (IPA, Qiagen).

Quantity of lipofuscin per cell. RPE cells were seeded in DMEM-10% FBS overnight. The following day media was replaced with Opti-MEM supplemented with indicated amounts of A2E (0, 10, 20, 30 μM) for 24 hours. A2E-loaded cells were dissociated with trypsin, counted and resuspended in lysis buffer. For eye RPE, 12-months-old WT and DKO mice were sacrificed, their eyes were enucleated and their RPE isolated from the neural retina and underlying choroid using RNA Protect Cell Reagent from Qiagen (Cat.no. 76526) at 100 uL/eyecup for 10 minutes, as previously described (Xin-Zhao Wang C et al., (2012) Exp Eye Res 102:1-9). Mouse RPE cells were counted and resuspended in lysis buffer. The lysis buffer used was 2% Triton X-100 1% SDS in water as it dissolved efficiently cell membranes and lipid-bisretinoids. The fluorescence of the lysates (arbitrary fluorescent units) was measured using a Spectramax M5s plate reader at 430 nm for excitation and 600 nm for detection.

Detection of lipid bisretinoids aggregation. To demonstrate aggregate formation of lipid bisretinoids, fluorescence of 500 μm A2E or ATRD solutions in PBS were determined before and after passage through 13 mm Nylon syringe disc filters 0.45 μm, and 3 μm (Tisch scientific, Ohio).

Immunoblotting. Total cell lysates were extracted using RIPA lysis buffer (50 mM TrisHCl, Ph 8.0, 150 mM NaCl, 1% NP40, 0.5% Sodium Deoxycholate, 0.1% SDS, protease-phosphatase inhibitor) after the incubation of the specific sample group in a 48 well plate. Sonication or syringing (20 times) was done followed by centrifugation at 12,000×g for 10 mins and the supernatant was collected and stored in −80° C. Proten concentration from homogenates were assessed by Pierce Rapid Gold BCA protein assay kit (ThermoFisher Scientific, cat.no. A53225). Then 30 μg of protein was analyzed by standard SDS-PAGE gel. Samples were mixed 4:1 (v:v) with Nupage loading buffer with or without 8% 0-mercaptoethanol and heated for 10 min at 72° C. SDS-PAGE was done using 4-12% Nupage gel and buffer (Life, NP0335BOX). Next, SDS-PAGE gel was transferred to nitrocellulose membrane (Whatman, PROTAN BA83) overnight at 20V. The following day, 5% milk in TBS was used for 2 h blocking the membrane and then primary antibody was added in TBST (TBS, 0.1% Tween-20) for overnight at 4° C. Next day membranes were washed and incubated for 2 hours at room temperature with HRP conjugated secondary antibody (cat #G21234, Invitrogen) at 1:10,000 dilution. After 3 more washed membranes were probed with enhanced chemiluminescence (ECL) reagent and detected using an X-ray films for chemiluminescence image (GE healthcare, RPN2106). Scanning/imaging and quantitation of the image was done using silverfast 8 application software and Fiji Image J. Antibodies for Western blotting were list in FIG. 10.

Retina ONL thinning quantification. Whole eyes were embedded in paraffin and sectioned at a thickness of 5 μm. Sections were counterstained with hematoxylin and eosin (H&E). Light microscopy was used to take digital images and the images were stitched by Zen software. Outer nuclear layer (ONL) thickness was measured at 0.2 mm intervals superior and inferior to the edge of the optic nerve head (ONH) along the vertical meridian. The center of ONH was used as the start of the measurement. The thickness of ONL was measured by Zen software.

Retinal RPE layer nuclei quantification. RPE nuclei was quantified in H&E counterstained cross sectioned eyecups. Images were taken at 40× and stitched by the Zen software. Number of RPE nuclei were counted every 0.1 mm intervals and plotted as a function of distance from ONH in 23 months old DKO (n=10) and 27 months old WT retinas (n=8). Mean values (±SEM) were significantly different at each point (p<0.05).

Retinal RPE cell size quantification. Retinal RPE eyecups were carefully dissected out of mouse eyes and stained with Alexa647-phalloidin for 1 hour at room temperature before flat-mounted on the slides. Using a Spinning Disk confocal microscope with a 63× lens, the RPE layers were imaged from the optic nerve head to the peripheral of the layer. The contours of individual RPE cells were visualized with phalloidin. Cell borders were manually selected using ImageJ software to calculate cell area and areas measured using ImageJ after manually selecting cell borders. Number of RPE nuclei were counted every 0.1 mm intervals and plotted as function of distance from ONH in 23 months old DKO (n=10) and 27 months old WT retinas (n=8). Mean values (±SEM) were significantly different at each point (p<0.05).

Statistics. All data were processed in Prism7.0 software. ANOVA, Student's t test or multiple t-test was used when appropriate. P values less than 0.05 were considered statistically significant.

### Example 2: LF Accumulation and Retinal Degeneration

HPLC and more recently quantitative fundus autofluorescence (qFAF) have become gold standards for measuring the content of LBs in retinas of animal models (Sparrow J R, et al. (2013) Investig Ophthalmol Vis Sci 54(4):2812-2820), yet the amounts reported by each method do not completely match. Experiments in mouse lines with excessive accumulation of LF, e.g Abca4^{-/-} or Abca4−/− RDH8−/− (double KO or DKO mouse) have reported a curious dichotomy: while qFAF indicates that the combined content of LBs in RPE and PRs increases continuously throughout life, HPLC shows a sharp decline in RPE's LBs after the first few months (Sparrow J R, et al. (2013) Investig Ophthalmol Vis Sci 54(4):2812-2820). This dichotomy was attributed to an early, sudden loss of RPE cells containing above threshold levels of LF that would permanently compromise the functionality of the epithelium promoting the formation and impairing the phagocytic removal of new LF in PRs (Flynn E, Ueda K, Auran E, Sullivan J M, Sparrow J R (2014) Invest Ophthalmol Vis Sci 55(9):5643-52).

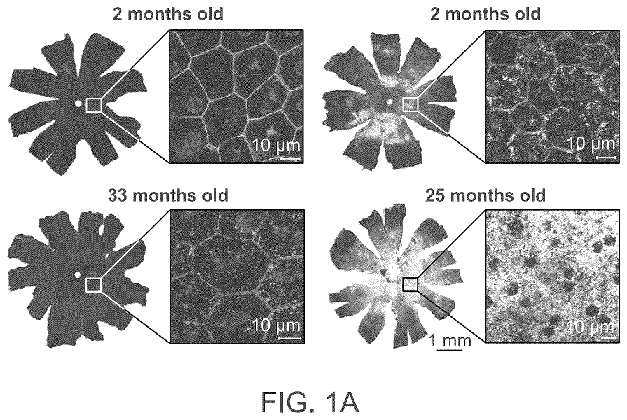

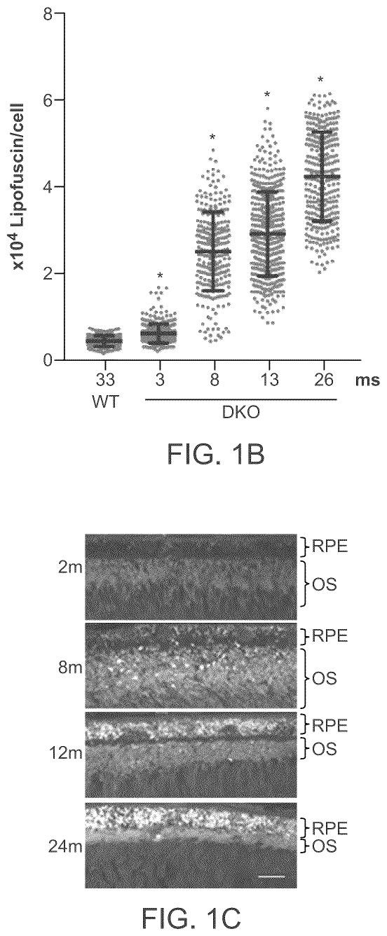

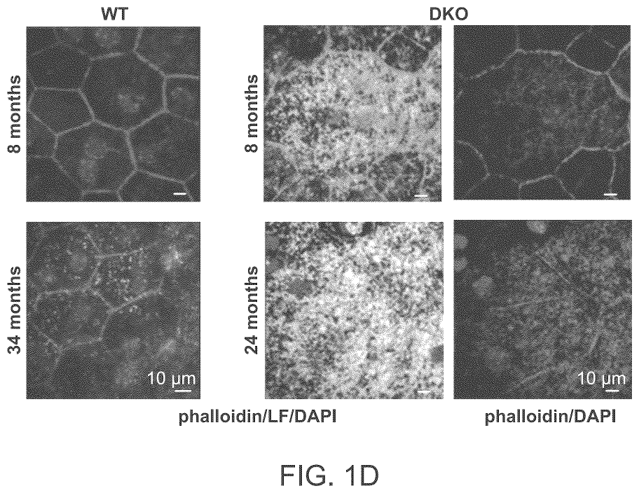

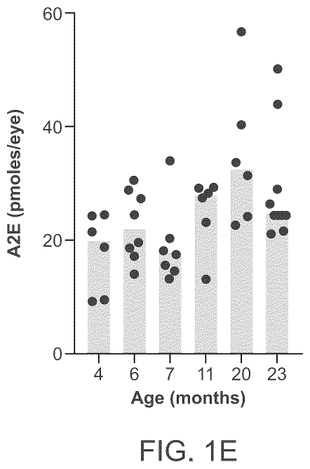

To elucidate the cytological and pathological aspects of this process, experiments in DKO vs control mice were carried out to compare the accumulation of HPLC-extractable LBs with the accumulation of fluorescent granules in the cytoplasm of RPE cells, measured by confocal microscopy, as a function of retina age. Using confocal microscopy, the number of LF-granules per RPE cell increased to fully occupy the whole cytoplasm in old DKOs (FIG. 1A). LF levels were 5-10× higher in DKO compared to old WT RPE cells (FIG. 1B). DKO's retina cross-sectional images revealed that the relative amount of LF between RPE and PRs did not appreciably change after the 8^{th} month, with the RPE remaining the main source of autofluorescence (FIG. 1C). Phalloidin staining to visualize the actin cytoskeleton and cell borders (FIG. 1D) revealed that WT RPE exhibited typical uniform hexagonal geometry that was maintained with age. In contrast, the organization of DKO RPE progressively deteriorated so that at 24 months, the prevalent phenotype comprised of scattered giant-multinucleated cells with the highest content of LF and intracellular stress fibers. The continuous accumulation of granules per RPE cell through life, comports with qFAF data, and supports the notion that the severity of LF burden gradually increases with aging instead of reaching a threshold that damages the RPE at younger ages. Additionally, the lipofuscin content of RPE cells was evaluated by high pressure liquid chromatography (FIG. 1E). The overall trend was a continuous increase of lipofuscin with age, in agreement with the confocal microscopy data.

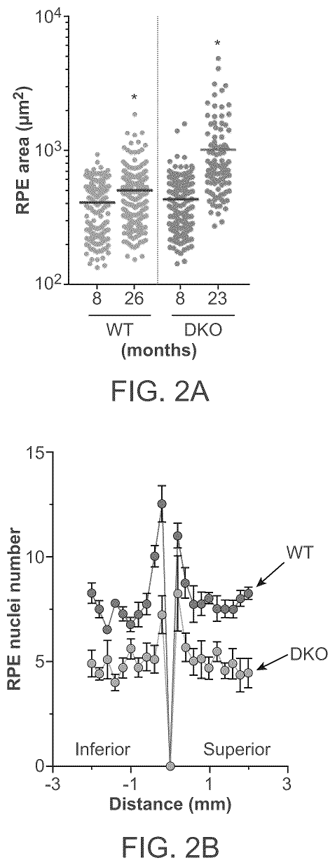

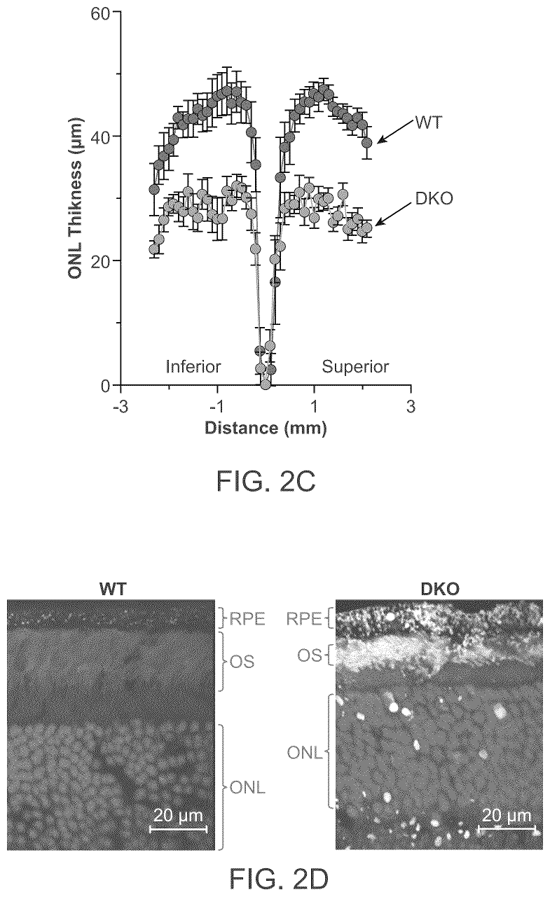

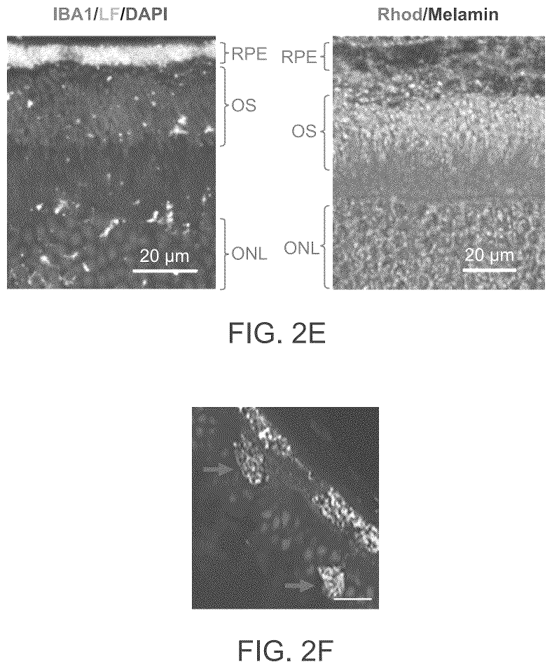

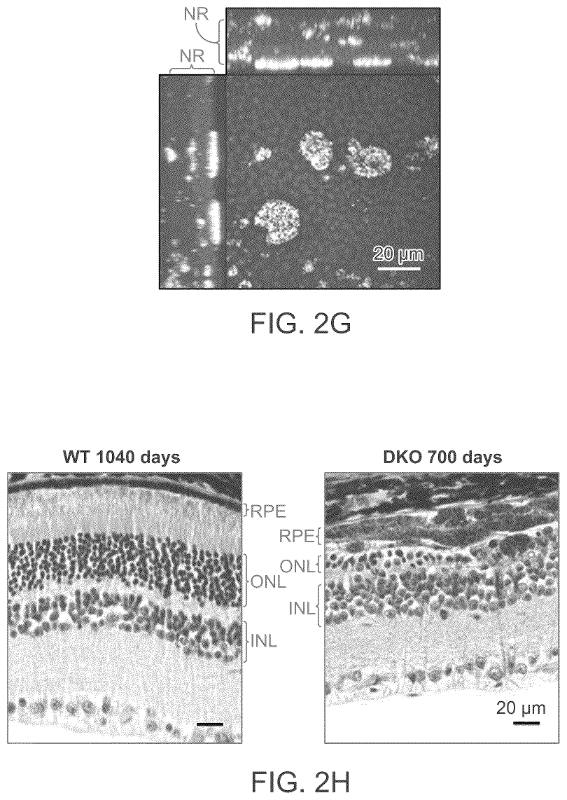

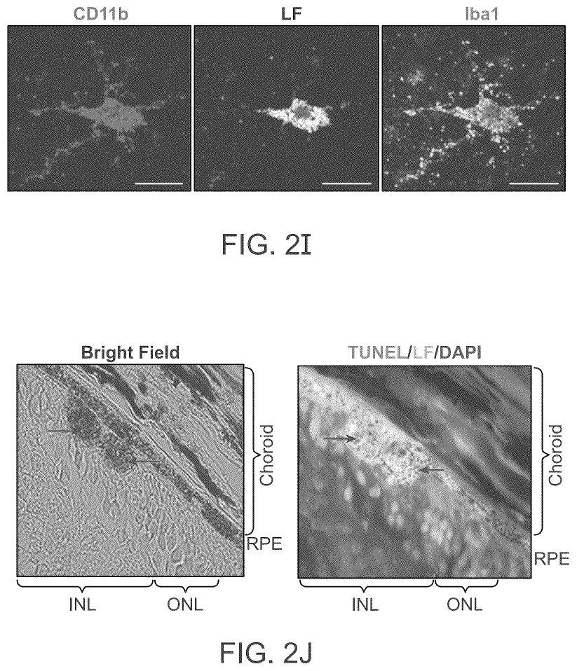

In order to quantify the extent of this phenomena, the size (area in μm²) of cells in random central locations using flat-mounts of DKO RPE detached from the neural retina between 8 and 23 months and age-matched controls (8 and 27 months) was surveyed (FIG. 2A). The average cellular size ranged from 324 to 411 μm² in WT and from 330 to 1000 μm² in DKO (p<0.01) demonstrating an important enlargement of RPE cells in retinas with LF. This correlated with a significant decrease in the number of RPE nuclei in DKO older than 25 months compared to age-matched WT (p<0.05) (FIG. 2B). The progressive retinal degeneration was also attested by the observation that 27 months old DKO mice had significant less PRs, from center to periphery, than same age-matched WT controls (FIG. 2C). Moreover, confocal microscopy of cryosections from old DKO retinas revealed the presence of small (˜1-3 μm) lipofuscin dots in the neural retina, which were absent in old WT controls (FIG. 2D). These particles stained positive for Iba-1, rhodopsin (FIG. 2E) and CD11b (FIG. 2I) but were negative for melanin (FIG. 2E) demonstrating that they represent activated microglia carrying phagocytosed pieces of degraded photoreceptor's outer-segments (FIG. 2E).

Furthermore, larger (˜5-10 μm) epithelial-sized fragments filled with LF (FIGS. 2F-2G) and melanin (FIG. 2H) in the neural retina were observed. These LF fragments may potentially represent migrating RPE cells/fragments undergoing epithelial mesenchymal transition (EMT); this observation could explain the origin of flecks and sloughed RPE characteristic of Stargardt and AMD retinas, respectively. Importantly, the outer nuclear layer (ONL) in close proximity to zones with RPE migratory activity appeared thin and disorganized (FIG. 2H), reflecting damage caused to photoreceptors (PRs) by the inflammatory process. Indeed, TUNEL staining on frozen sections demonstrated dead PRs around the migratory RPE fragments (FIG. 2J). Overall, these results demonstrate a direct connection between accumulation of LF granules in the RPE and accelerated erosion of the retinas which may be due to direct LF toxicity as well as the recruitment of activated microglia and the promotion of EMT behavior in the RPE.

Lipofuscin was quantified in mouse RPE cells in DKO and WT mice of different ages by microscope. Mouse RPE cells were photographed from the center (ONH) to the periphery of mouse eyecup. The lipofuscin of central RPE cells was quantified by Image J and graphed. Over 300 RPE cells were quantified in each group, each dot in the graph represent a single cell. The lipofuscin contain in DKO 8 months, 13 months and 26 months are much higher than DKO 3 months (p<0.01 by unpaired t test). DKO 3 months is higher than WT 8 months and 33 months (p<0.01 by t test). WT 33 months group is higher than WT 8 months group with significance (p<0.01 by unpaired t test).

Taken together, confocal images of RPE from DKO eyecups showed that the number of LF granules per cell increased uninterruptedly with age (FIGS. 1A-1D) which was accompanied by the enlarged size and reduced number of RPEs, reflecting the expansion of surviving cells to seal the gaps generated by the death or migration of RPEs into the neural retina. The observed RPE death could result from direct toxicity or indirect damage caused by the activated microglia/macrophages recruited into the layers with LF (FIGS. 2A-2H). However, the content of LBs in the RPE dropped significantly after the 8^{th} month of life (FIGS. 2I-2J) while the number of granules per cell kept increasing. This dichotomy may be attributable to the incremented chloroform insolubility of the content of LF-granules associated with aging, rather than the loss of LF-laden RPE.

### Example 3: LF Photooxidation and Retinal Degeneration

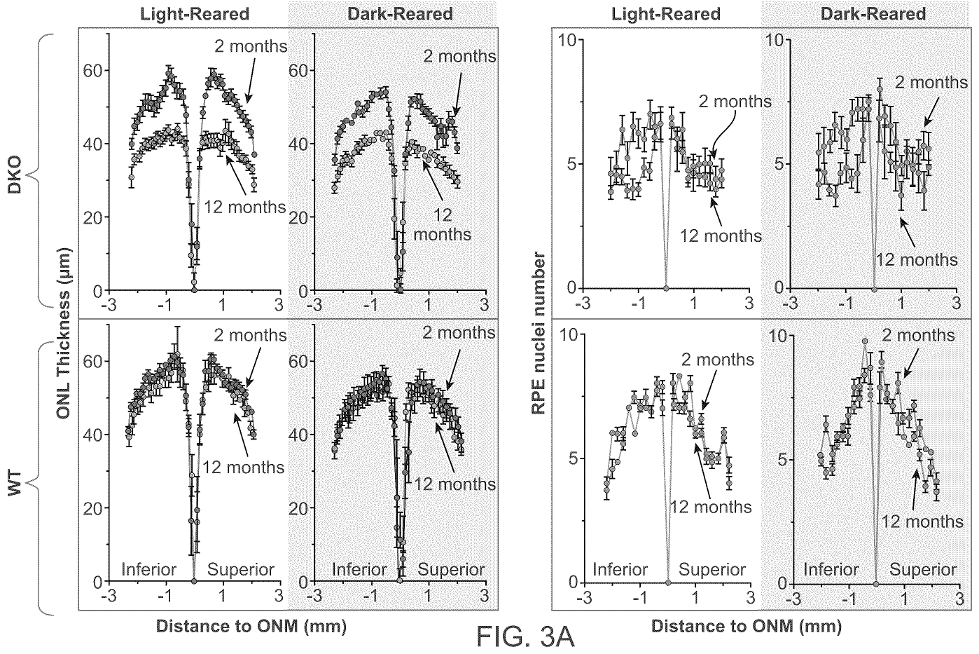

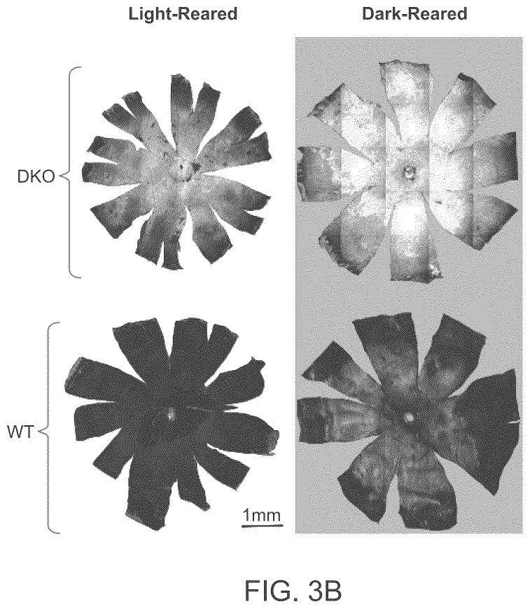

Photooxidative degradation of LBs contributes to retinal degeneration in albino animals, but it remains unclear whether photooxidation takes place at significant levels in pigmented retinas. Since photooxidation can be stopped by rearing animals in complete darkness (Ueda K et al., (2016) Proc Natl Acad Sci USA. 113(25):6904-9, Boyer N P et al., (2012) J Biol Chem 287(26):22276-22286) without affecting the accumulation of new LBs, studies were performed to determine whether RPE and PRs would still degenerate in DKO retinas never exposed to light. Accordingly, WT and DKO pigmented mice were housed from birth in either continuous darkness or under 12 h cyclic light conditions. The change in the thickness of their ONL as well as the number of RPE nuclei were evaluated for up to one year. As shown in FIG. 3A, LF-associated thinning of the ONL and reduction of RPE number proceeded at similar rates independently of whether the mice were light-cycled or dark reared. WT groups did not show significant loss of PRs or RPE during the same period irrespective of illumination conditions. To verify that LBs still accumulate in the absence of light, LF autofluorescence was quantified in each of the four groups in RPE flat mounts from 12 month old animals (FIG. 3B). Dark-reared mice, both WT and DKOs, contained ˜2.8 times more auto-fluorescent material than their respective counter parts under cyclic light conditions (p<0.05) and DKOs contained ˜5 times more LF than WTs (FIG. 3B). These results revealed that in vivo light-independent toxic cascades contribute to the deterioration of the RPE and PRs in pigmented retinas with excessive LF levels.

Taken together, the comparable loss of PRs and RPE cells between dark- and light-reared DKO retinas (FIG. 3A) and the significant photobleaching of the LF autofluorescence in eyes exposed to light (FIG. 3B) show that the intrinsic toxicity of the granules, independently from their tendency to photooxidize, plays a role in the degeneration process, while suggesting that pigmented retinas may be able to handle moderate levels of LB photooxidation.

### Example 4: Light-Independent Cell-Death In Vitro

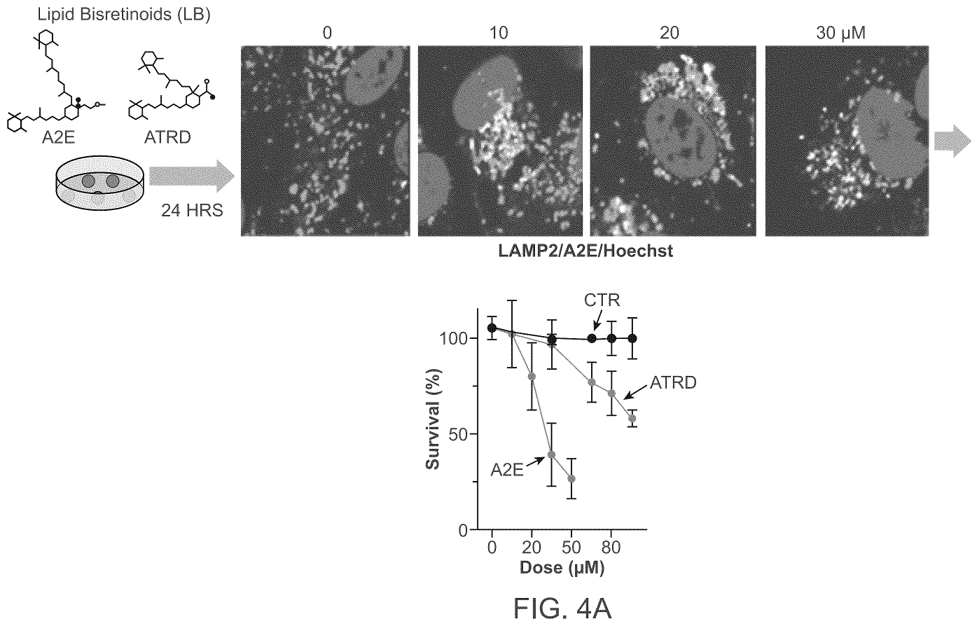

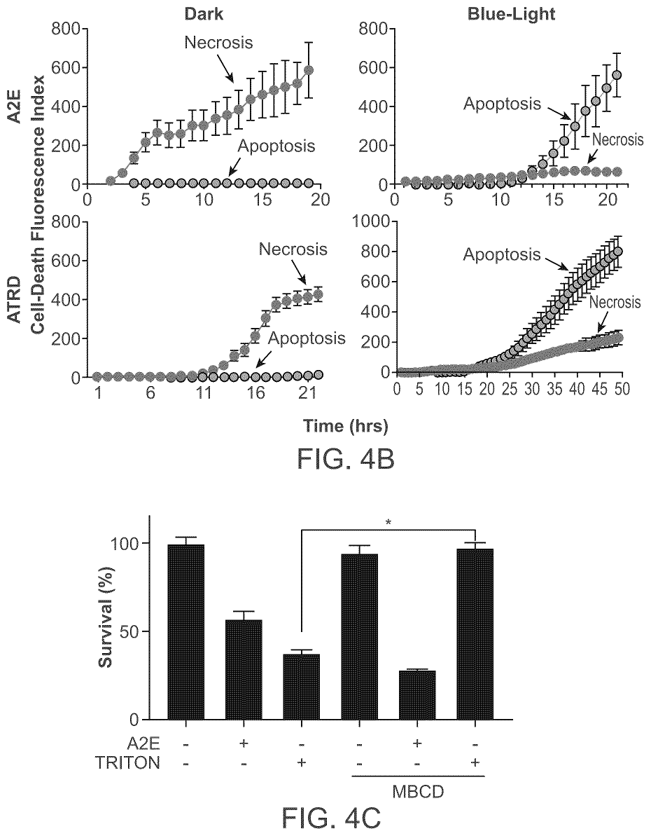

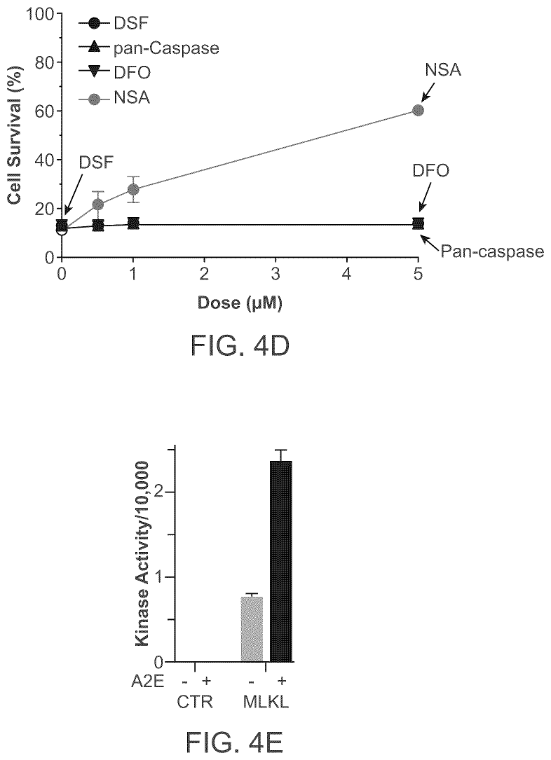

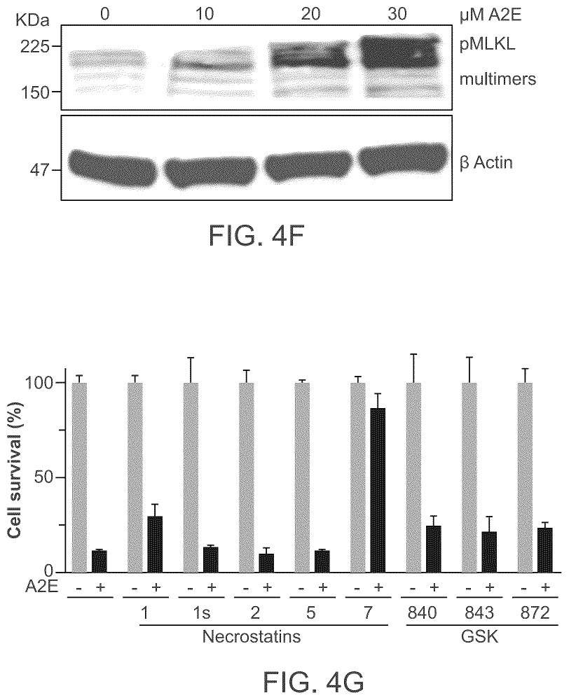

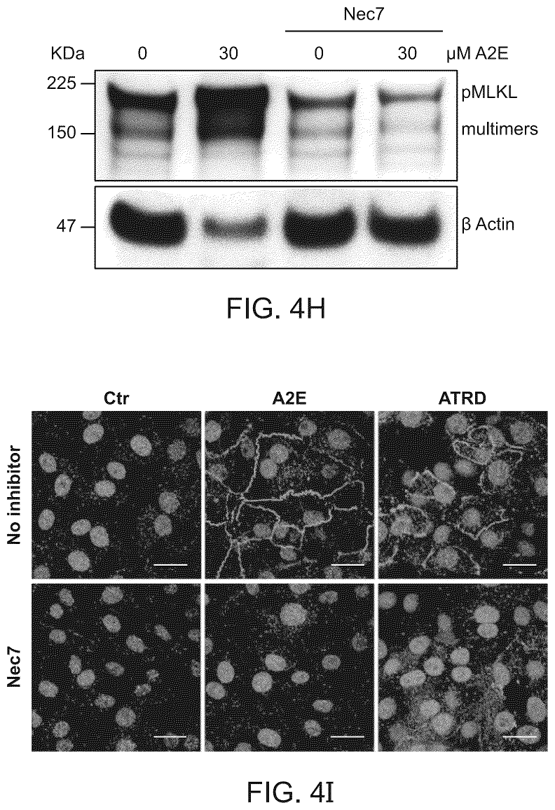

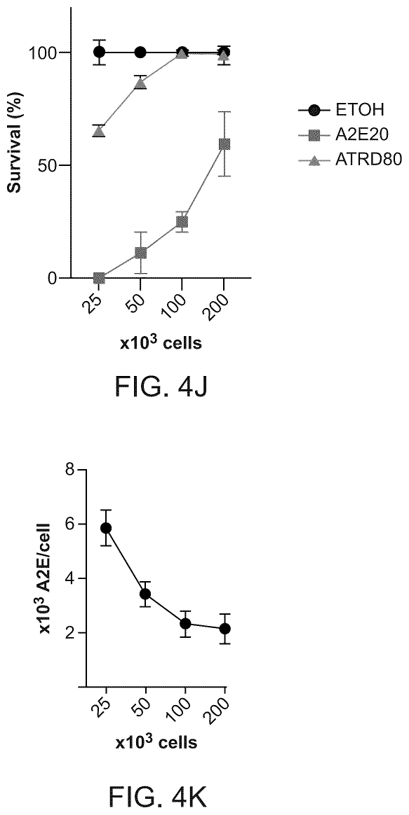

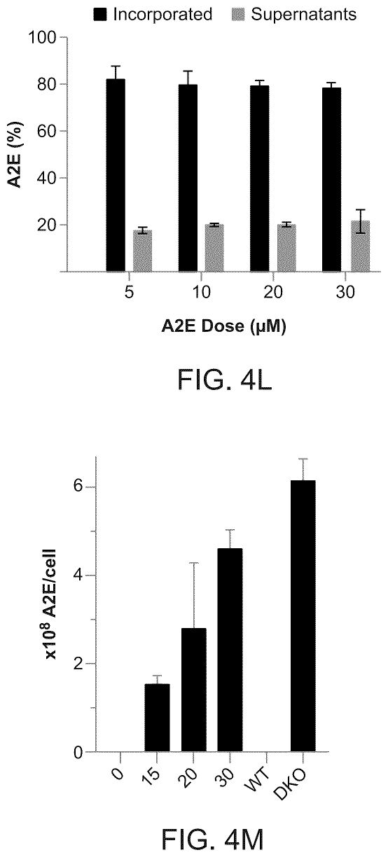

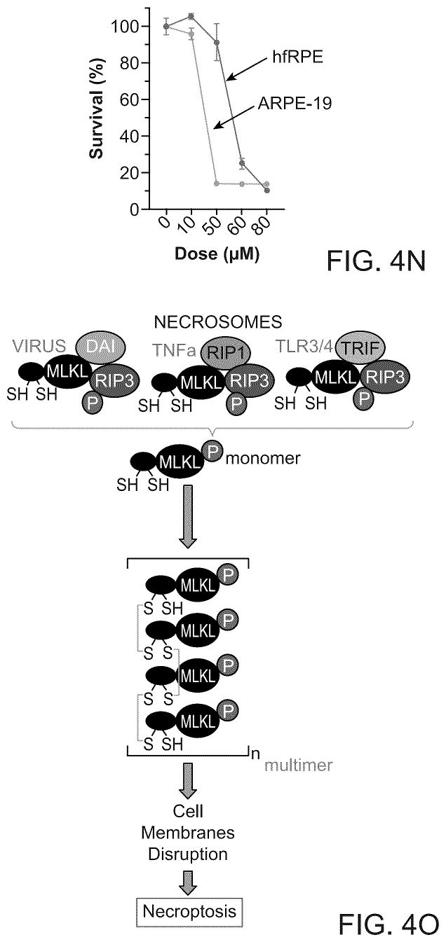

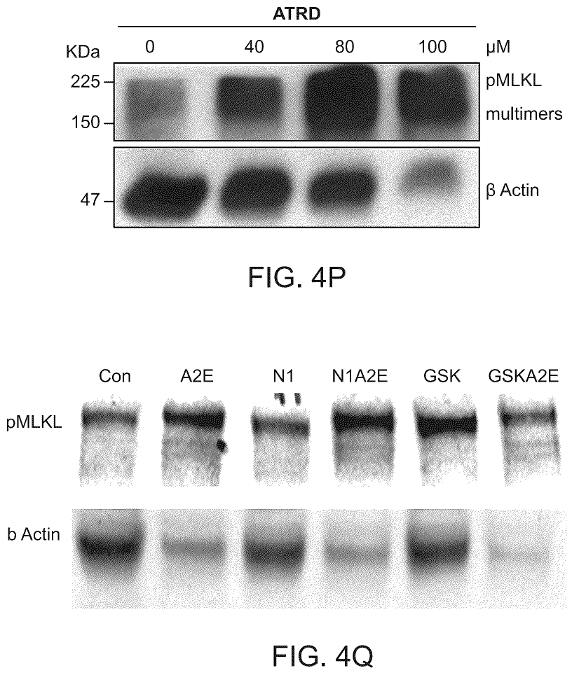

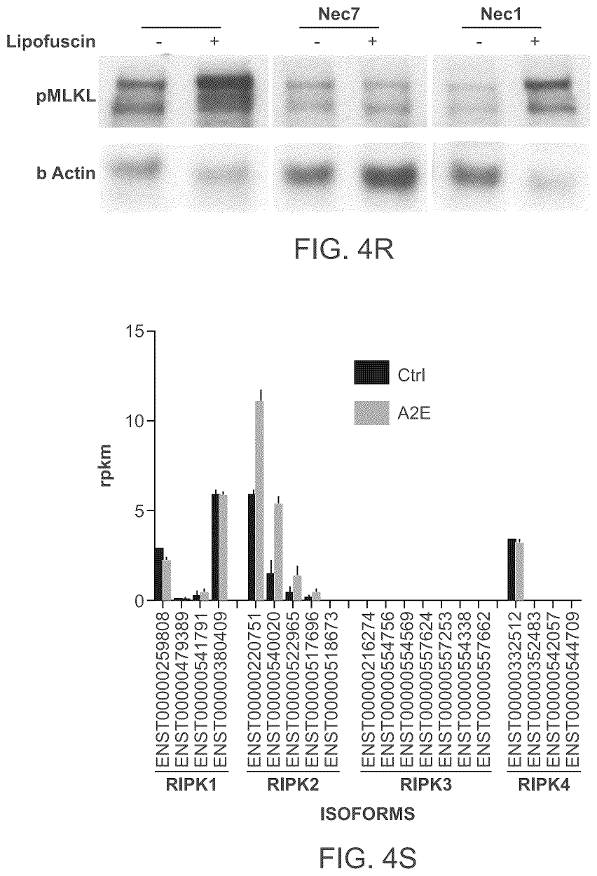

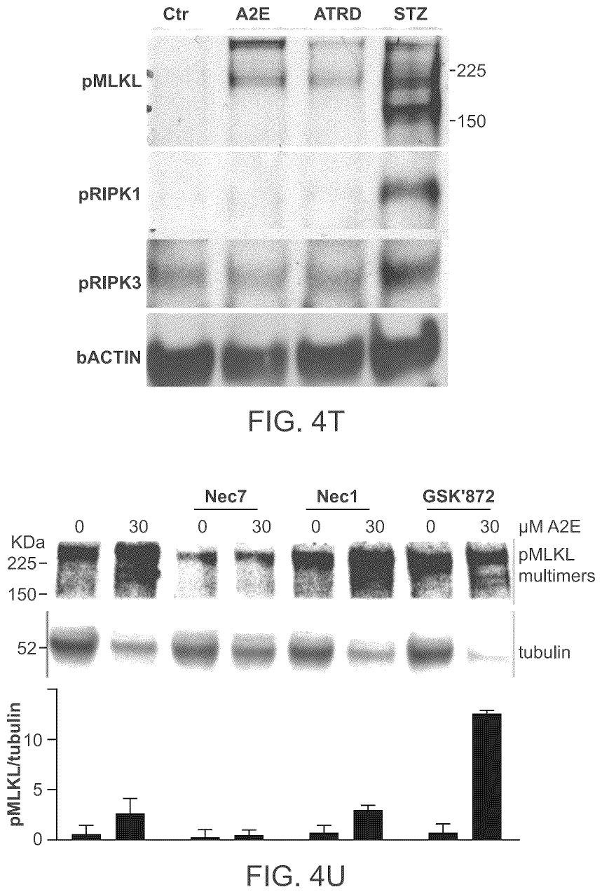

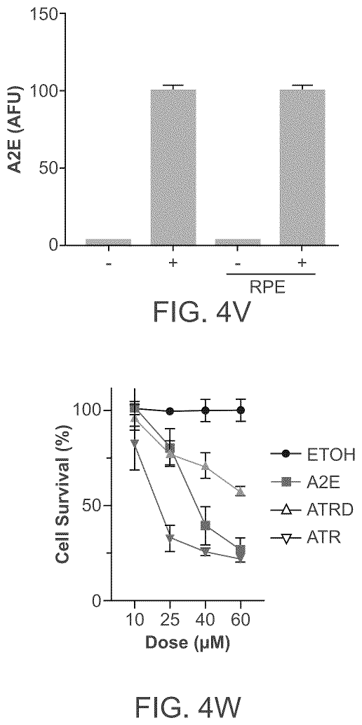

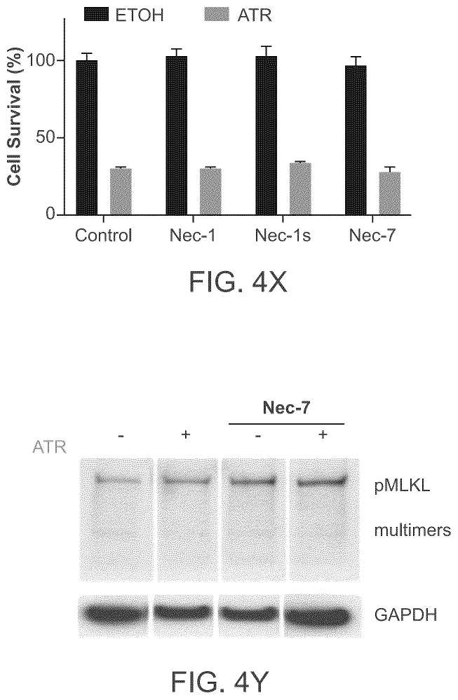

To study the mechanisms involved in light-independent cell death, existing protocols of incorporating LBs into the lysosomes of RPE cells were adapted (Sparrow J R et al., (1999) Invest Ophthalmol Vis Sci 40(12):2988-95, Sparrow J R, Kim S R, Wu Y Chapter 18 Experimental Approaches to the Study of A2E, a Bisretinoid Lipofuscin Chromophore of Retinal Pigment Epithelium. 315-327, Boulton M E (2014) Exp Eye Res 126:61-7) and dose dependent RPE cell-death under no light condition was reproducibly provoked (FIG. 4A).

The susceptibility to LF depended heavily on cell confluency (FIG. 4J) as denser cell cultures were more resistant to incorporate LBs (FIG. 4K). The efficiency of incorporation was nonetheless constant for the different LB-doses (FIG. 4L). As default, 80% confluency was chosen for the assays because under these conditions ARPE-19 took up 80% of supplemented LBs reaching LB levels within the concentration range found in the RPE of DKO retinas (FIG. 4M). Importantly, the fluorescence reads of lysates from ARPE19 and pigmented RPEs from retinas were not affected by the melanin (FIG. 4V). Using this in vitro setup, LF caused also light-independent cell-death in highly differentiated hfRPE (FIG. 4N).

LF-induced apoptosis and necrosis at single-cell level was investigated by adding NucView®405 (a non-fluorescent cell-permeant substrate that stains nuclear DNA blue when cleaved by caspase-3 during the executioner phase of apoptosis) and DRAQ7 (a dye that stains DNA red only if cells have compromised membrane integrity) to the cultures. LF induced necrosis in the absence of light, but if exposed to blue-light, apoptosis was induced (FIG. 4B). ATRD was a less potent inducer of necrosis and apoptosis than A2E, although it had the same aldehyde group touted as responsible for the high toxicity than its precursor, all-trans-retinal (ATR) (Maeda A et al. (2012) Nat Chem Biol 8(2):170-8). On the other hand, A2E but not ATRD contains both hydrophobic retinoid-derived chains and a hydrophilic pyridinium head group that conferred amphiphilic properties (Soma De S, Sakmar T (2002) J Gen Physiol 120(2):147-157).

To determine whether A2E induces necrosis by intercalating into membranes, methyl beta-cyclodextrins (MβCD) was used. MβCD is a cyclic sugar that protects against detergent effects by forming soluble complexes with amphipathic molecules. Remarkably, although MβCD complexed with both A2E and Triton-X100, it did not protect against A2E but fully shielded against lethality caused by Triton-X100 (FIG. 4C). This result along with the slow kinetics of cell death (several hours vs instantaneous with Triton-X100) suggested that LF triggered programmed necrosis. Thus, the protective effects of inhibitors of two main effector cascades of regulated necrosis: caspase/gasdermin-D and RIPK3/MLKL were tested (FIG. 4D).

Pretreatment with the pan-caspase inhibitor z-VAD(OMe)-FMK and the gasdermin-D inhibitor disulfiram provided no protection. GSK'872, a selective inhibitor of RIPK3 (the only known kinase to phosphorylate human MLKL at Ser358 (pMLKL)) did not protect, either.

However, dabrafenib an ATP competitive inhibitor of B-Raf and RIPK3 or necrosulfonamide (NSA), a drug that prevents the spontaneous assembly of pMILKL into oligomeric pores that insert into membranes that eventually kill, increased RPE survival in a dose dependent manner. For its phosphorylation, MLKL and its kinase need to be recruited into multiprotein complexes, known as necrosomes (FIG. 4O). Necrosomes are not well conserved among species and vary also with the nature of the stimuli that triggers their formation. Anti-MLKL immunoprecipitation (IP), yielded pulldowns with kinase associated activity only when prepared from lysates coming from cells containing LF, demonstrating that MLKL was part of a necrosome (FIG. 4E). Considering that phosphorylation of human MLKL at Ser358 (pMLKL), is a hallmark triggering event and marker of necroptosis, the Ser358-phosphorylation and polymerization status of MLKL in cells with LF was determined. Using Western Blotting (WB), under non-reducing conditions to preserve and increase the chances of detecting phospho-oligomers, it was demonstrated that A2E (FIG. 4F) as well as ATRD (FIG. 4P) cause dose-dependent phosphorylation and polymerization of MLKL in the absence of light. WB analysis of anti-MLKL pull downs (Co-IP) revealed no bands corresponding to RIPK1 or RIPK3, the most common partners in the necrosome (data not shown). A prerequisite for the assembly of these RIP kinases into necrosomes was their phosphorylation.

To rule out that the low expression of these proteins in ARPE19 precluded their detection, experiments were performed in human intestinal HT29 cells after treatment with LBs or TNFα/caspase inhibitors (FIG. 4T). Although HT29 cells expressed high levels of these proteins, pRIPK1 and pRIPK3 were still not detectable in cells with LF but were readily visible upon treatment with the TNFα inhibitor cocktail. The spectrum of protection offered by the necrostatins was also investigated. Necrostatins were initially identified for their powerful inhibition of TNFα induced necroptosis in FADD deficient Jurkat T cells (Zheng W, Degterev A, Hsu E, Yuan J, Yuan C (2008) Bioorganic Med Chem Lett 18(18):4932-4935, Teng X, et al. (2005) Bioorganic Med Chem Lett 15(22):5039-5044). Necrostatin-1 (Nec1), Nec1s, Nec2 and Nec5 all target RIPK1 (Degterev A, et al. (2008) Nat Chem Biol 4(5):313-321), while Nec7 targets an unknown regulatory molecule in the pathway. Accordingly, RIPK1-targeting necrostatins provided no survival benefit, while Nec7 was highly protective (FIG. 4G). WB results show that while treatment with Nec7 (FIG. 4H and FIG. 4U) reduced LF-induced phosphorylation and polymerization of MLKL, Nec1 or GSK'872, did not (FIG. 4U).

In contrast, Nec1 did not block phosphorylation and polymerization of MLKL (FIG. 4R). Finally, using confocal fluorescence microscopy, Ser358 phospho-HLKL was shown to localize into membranes in cells with aberrant levels of A2E and ATRD, but not in healthy controls (FIG. 4I) and Nec7 treatment effectively reverted the pMLKL membrane localization pattern. Collectively, these results demonstrate that LF induces a novel and uncharacterized type of necrosome that does not contain RIPK1 and potentially lacks RIPK3. Chronic accumulation of polymeric pMLKL could account for progressive damage in pigmented retinas as they amass LF.

These results demonstrate that the light-independent cytotoxicity of A2E or ATRD loaded LF-granules elicited necroptosis, which is relevant to the pathobiology of GA. Necroptosis is a type of programmed cell-death that leads to cell membrane disruption causing atrophic areas and the release of cellular constituents known that elicit local inflammation. Evidence for light-independent LF mediated necroptosis is as follows: (i) real-time monitoring of cell death showed early impairment in membrane integrity without caspase-3 activation; (ii) LF induced a dose-dependent Ser358-phosphorylation, polymerization and plasma membrane translocation of the pseudokinase mixed-lineage kinase domain-like (MLKL), in light-free conditions; (iii) cell-death was preventable with dabrafenib and NSA; (iv) Immunoprecipitation experiments revealed that LF promoted the association of MLKL with a kinase, indicative of necrosome formation; (v) dark cell-death could not be prevented by the pan-caspase inhibitor z-VAD(OMe)-FMK nor anti-oxidants; and (vi) Nec7, potently prevented MLKL phosphorylation, polymerization, plasma membrane localization and cell-death by LF. See FIGS. 4A-4I.

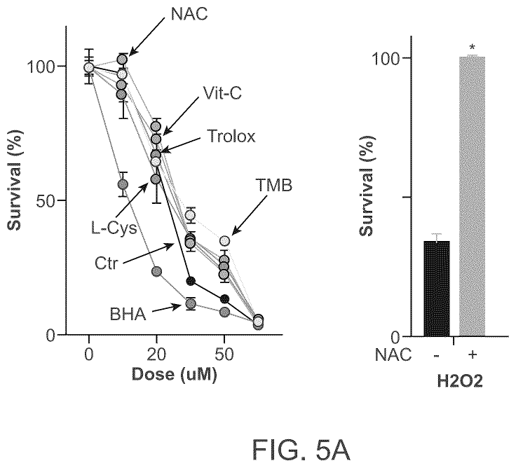

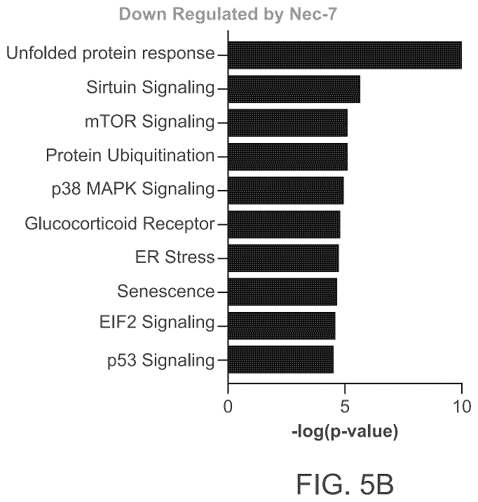

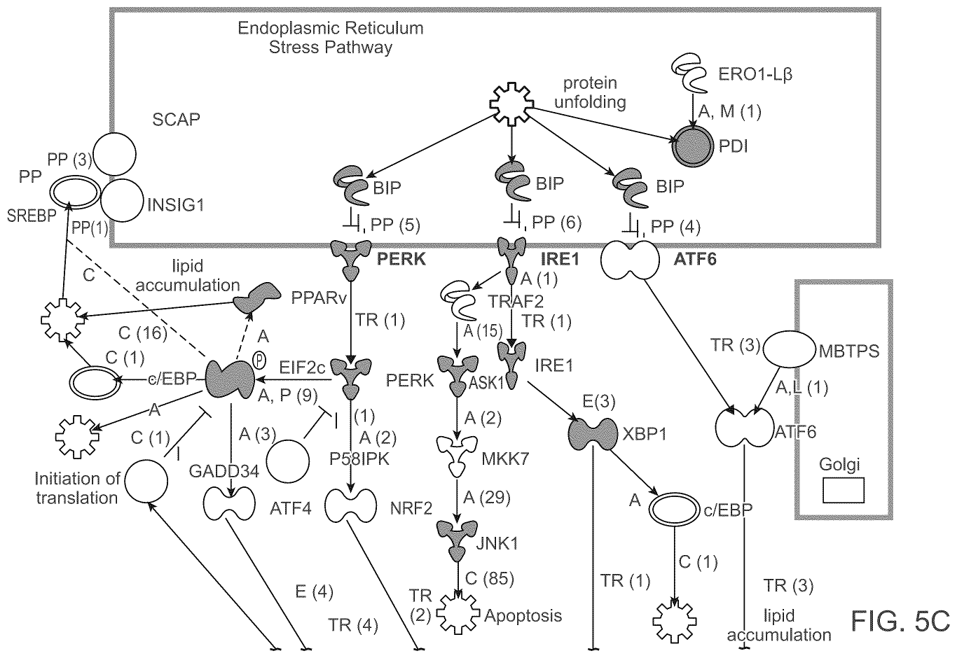

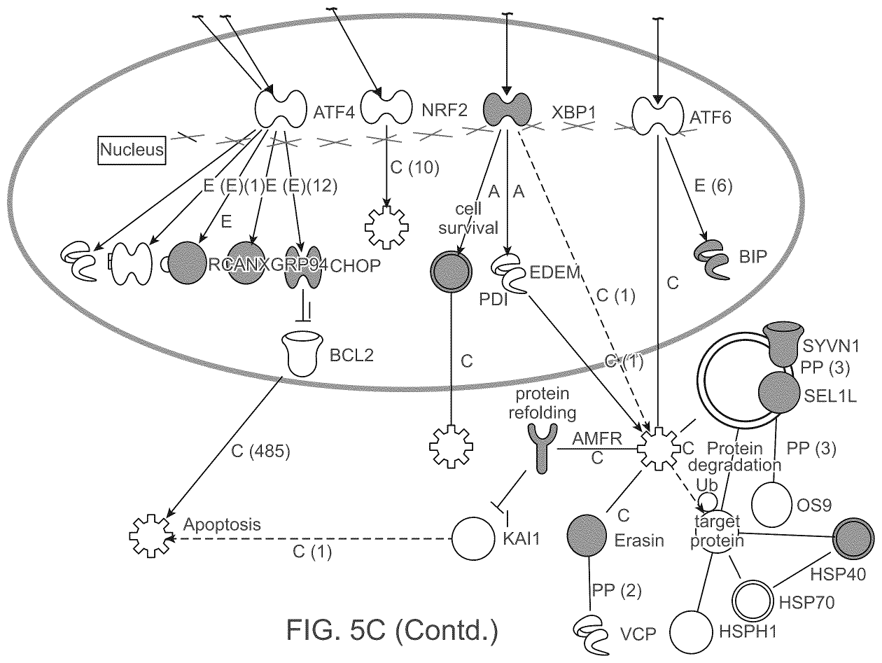

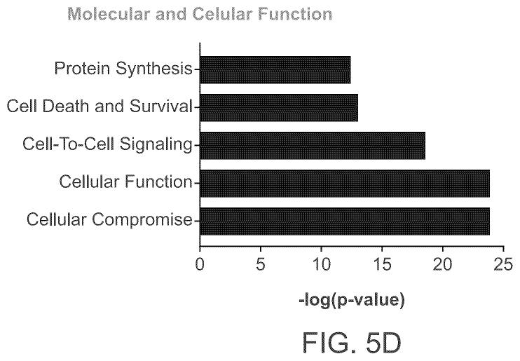

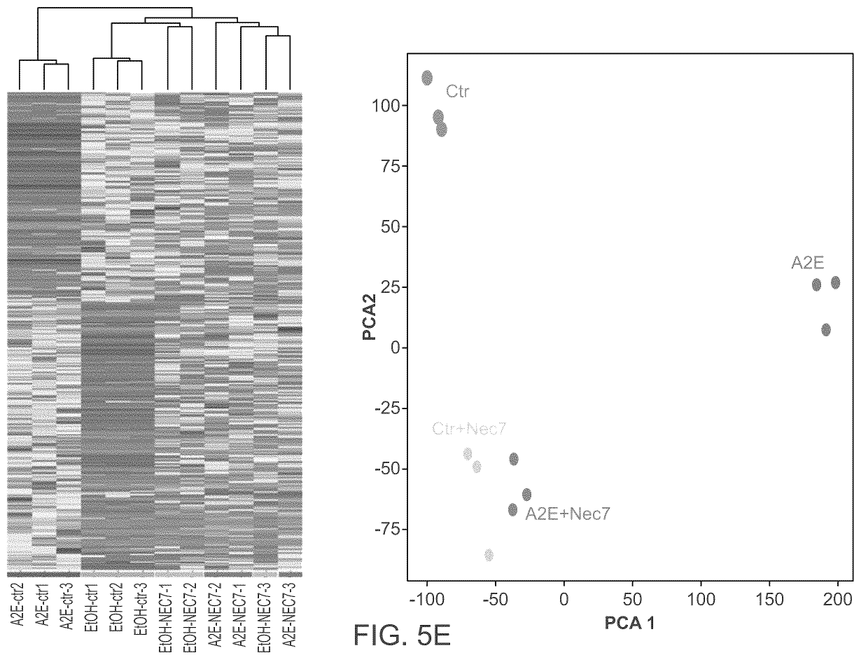

LF cell-death and MLKL phosphorylation/polymerization were not affected by GSK'872 (FIG. 4D and FIG. 4Q) and RIP1 kinase inhibitors Nec1, Nec1s, Nec2 and Nec5 (FIG. 4G and FIGS. 4Q-4R) and was insensitive to antioxidants (FIG. 5A). Moreover, RIPK3 was undetectable at mRNA and protein level in ARPE19 and hfRPE cells, even after pro-necroptotic LB treatments (FIG. 4S). In cells with high levels of expression of RIPK1 and RIPK3, LF still did not induce detectable phosphorylation of these molecules, while the classical combination of TNFα plus Smac mimetic and the caspase inhibitor z-VAD (TSZ) that is used to induce RIPK1-RIPK3-mediated necroptosis, clearly did (FIG. 4T). All these combined results demonstrate that RIPK1 and RIPK3 are not part of the LF-induced necrosome.

Finally, since it has been suggested that apart from the adverse effects of lipid-bisretinoids, the retina of ABCA4^{-/-}RDH8^{-/-} mice succumbs from the toxicity of the all-trans-retinal (ATR) released upon illumination, the link between ATR and necroptosis was investigated. Using the cell-death assay, ATR was found to trigger a light-independent cell-death in ARPE19 cells (FIG. 4W), but the mechanism was different from the canonical or lipofuscin-elicited necroptosis as it was insensitive to Nec1 as well as Nec7 (FIG. 4X). Western Blot of cells treated with ATR showed no increase in phosphorylation/polymerization of MLKL (FIG. 4Y). Overall, these indicate that necroptosis is a specific readout of ocular lipofuscin toxicity, easily distinguishable from the poisonous effects of ATR.

These results demonstrate that Dabrafenib, necrosulfonamide (NSA), Necrostatin 7 (Nec7), and IRE1α inhibitors that block IRE1α dimerization are useful in methods for preventing or treating an eye disease associated with retinal cell lipofuscin-associated cytotoxicity in a subject in need thereof.

### Example 5: LF Triggers MLKL-Induced Necroptosis

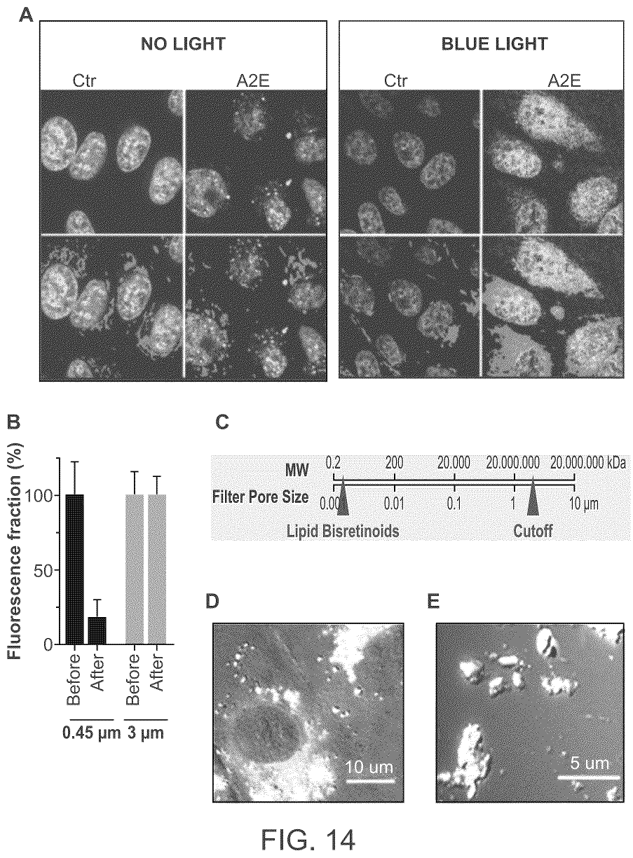

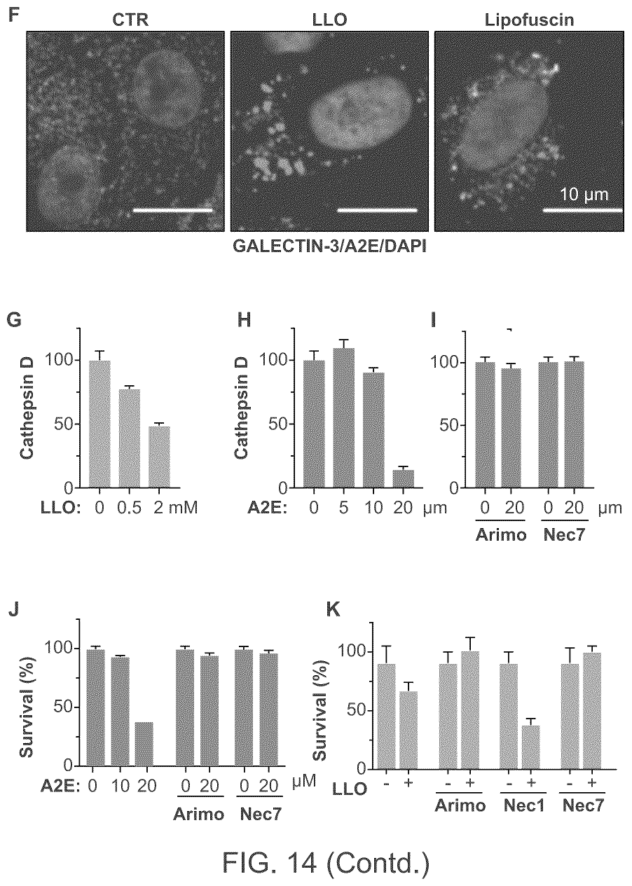

LF deposits are thought to induce oxidative stress (Ueda K, et al. (2018) Proc Natl Acad Sci USA 115(19):4963-4968). Thus, the protective effects of a variety of antioxidants: N-acetyl cysteine (NAC), Trolox, L-cysteine, vitamin C, BHA and TMB against LF cytotoxicity was investigated. None of the tested antioxidants had any effect on RPE survival relative to LF accumulation, although NAC effectively prevented oxidative damage by H₂O₂ (FIG. 5A). Additionally, reactive oxygen species (ROS) at subcellular levels was visualized with CellROX Deep-Red®. In the dark, cellular ROS appeared associated only with mitochondria and not in lipofuscin-granules. However, when cells were illuminated with blue light, the LF-granules busted and the auto-fluorescent material became cytosolically spread and positive for ROS (FIG. 14A). Accordingly, even though the method could effectively detect LF-induced ROS generation, there was no evidence of oxidative stress associated with the granules during the induction of necroptosis.

Since accumulation of crystalline materials in cells can elicit necroptosis and atomic force microscopy revealed that the core of lipofuscin granules comprises solid aggregates, the ability of lipid bisretinoids to form crystals that kill cells was investigated. A2E (MW 592 Da) in aqueous milieu formed aggregates the size of a bacteria, that could not move across 450 nm filter pores (FIGS. 14B and 14C). This inability to pass was due to size exclusion and not to non-specific binding, as A2E did cross membranes with 3 μm cut-off of the same material. Next, A2E buildups were imaged using a combination of DIC and fluorescence: A2E appeared as granules with well-defined edges, both within cells or after drying over a coverslip (FIGS. 14D and 14E). Next, the ability of the suspected A2E crystals to damage lysosomal membranes was investigated. A recently developed galectin-3 puncta assay for early detection of lysosomal membrane permeabilization (LMP) was used. Briefly, cytosolic galectin-3 rapidly binds to the glycocalyx in the luminal face of lysosomal membranes as they become leaky, which is easily detectable with anti-galectin high-affinity antibodies. L-Leucyl-L-Leucine methyl ester (LLO) a lysosomotropic peptide that causes LMP was used as positive control of puncta formation (FIG. 14E). 50 μM A2E buildups also clearly caused puncta staining and therefore LMP (FIG. 14F). LMP was confirmed by showing the inactivation of cathepsin D, as surrogate of lysosomal enzymes. Both LLO (FIG. 14G) and A2E (FIG. 14H) caused reduction in cathepsin-D activity. Interestingly, loss of cathepsin-D was prevented by arimoclomol, a drug that promotes lysosomal integrity through the upregulation of heat shock proteins, as well as by Nec-7, the only necrostatin that protects against lipofuscin (FIG. 14I). Furthermore, arimoclomol like Nec-7 protects against necroptosis by lipofuscin (FIG. 14J). Also, very remarkable was the observation that the same unique pattern of protection with arimoclomol and Nec-7 but not Nec-1 was observable for LLO (FIG. 14K), consistent with the idea that necroptosis by A2E is being caused by LMP.

To investigate the cellular processes leading to necroptosis, Ingenuity Pathway Analysis (IPA) was used to compare the transcriptomes of cells suffering of dark LF cytotoxicity with cells rescued by Nec7 treatment. As shown in FIG. 5B, inhibition of the unfolded protein response (UPR) was by far the most significant variation induced by the treatment with Nec7. The UPR is a conserved transcriptional response to the aberrant accumulation of misfolded proteins or lipids in the endoplasmic reticulum. UPR is elicited by three ER-resident sensors: PERK, IRE1α and ATF. Visualization of the UPR in IPA revealed that PERK and IRE1α pathways were negatively regulated by Nec7 at multiple points (FIG. 5C). To evaluate the impact of UPR downregulation, the list of UPR transcripts inhibited by Nec7 was extracted and a new IPA analysis was performed for the downstream consequences (FIG. 5D). UPR had a significant impact on cell-death along with other cellular functions and communication signals.

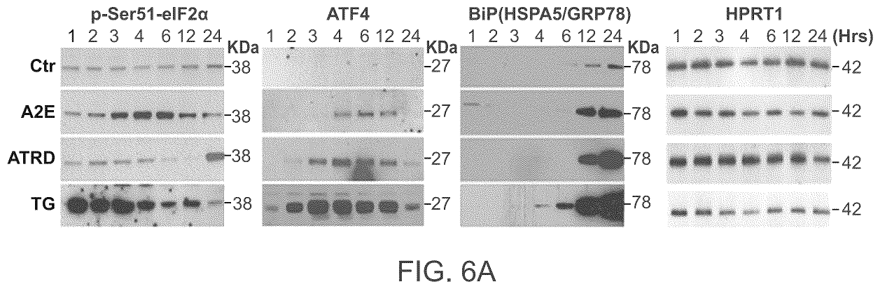

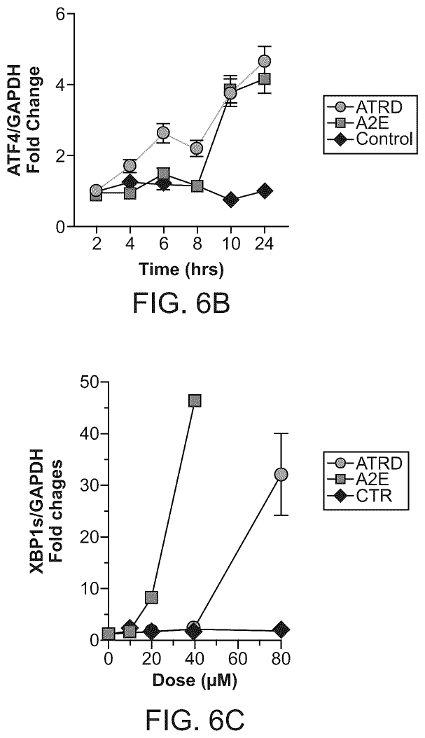

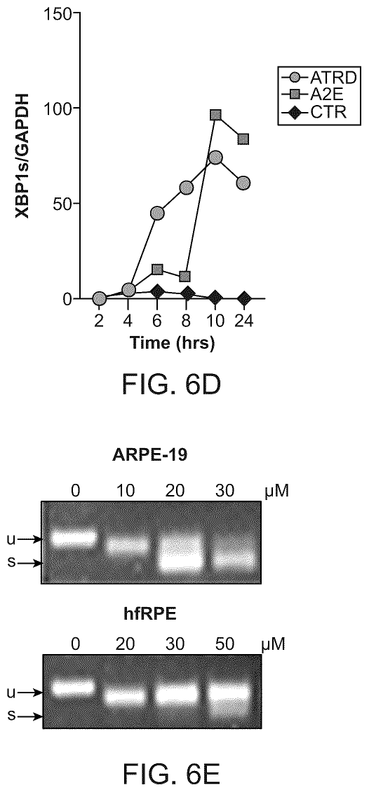

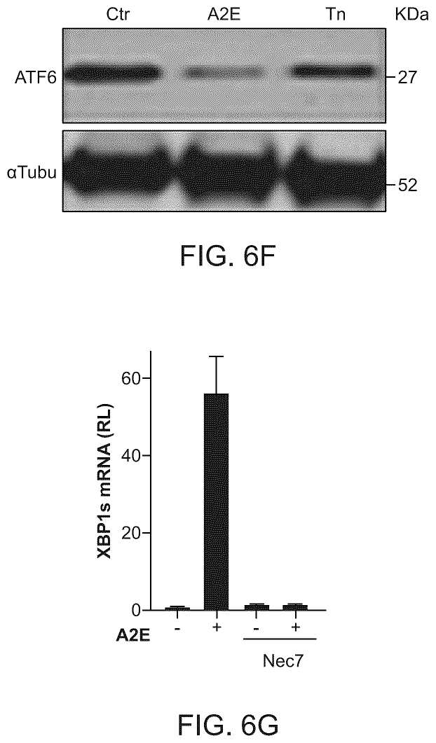

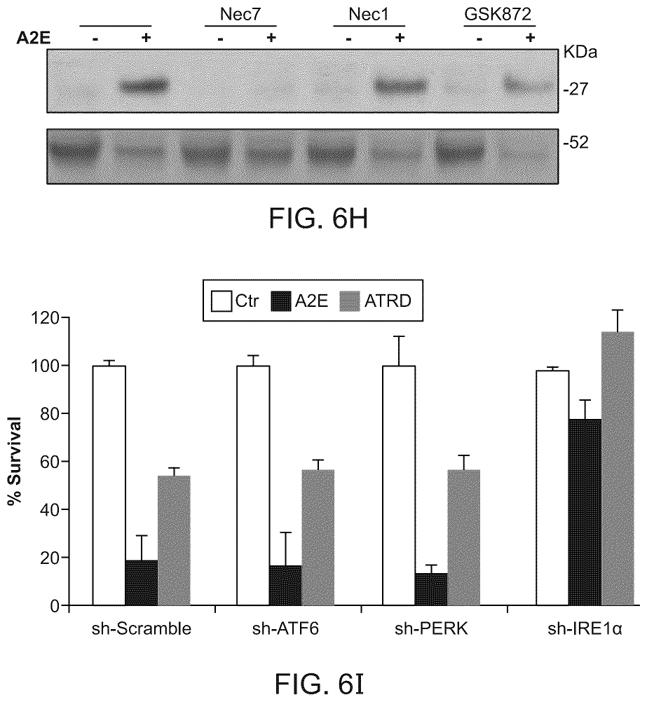

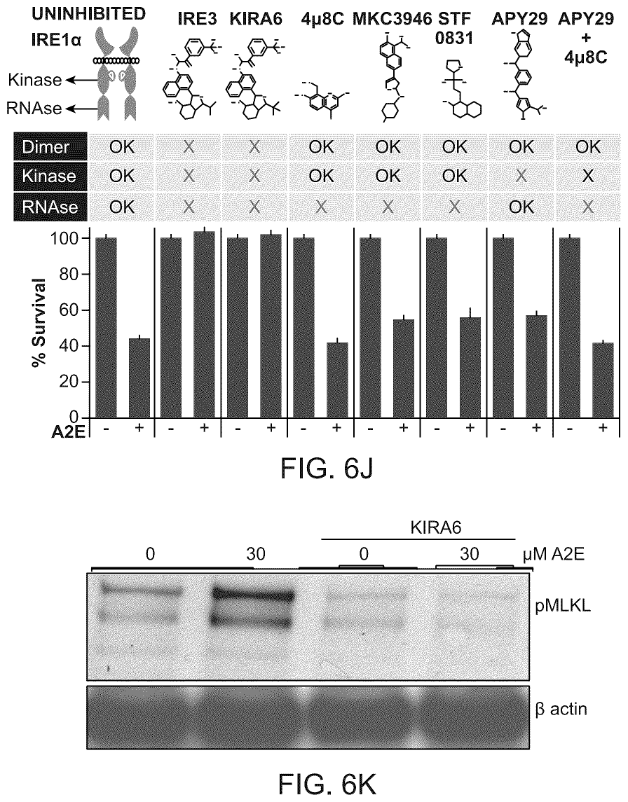

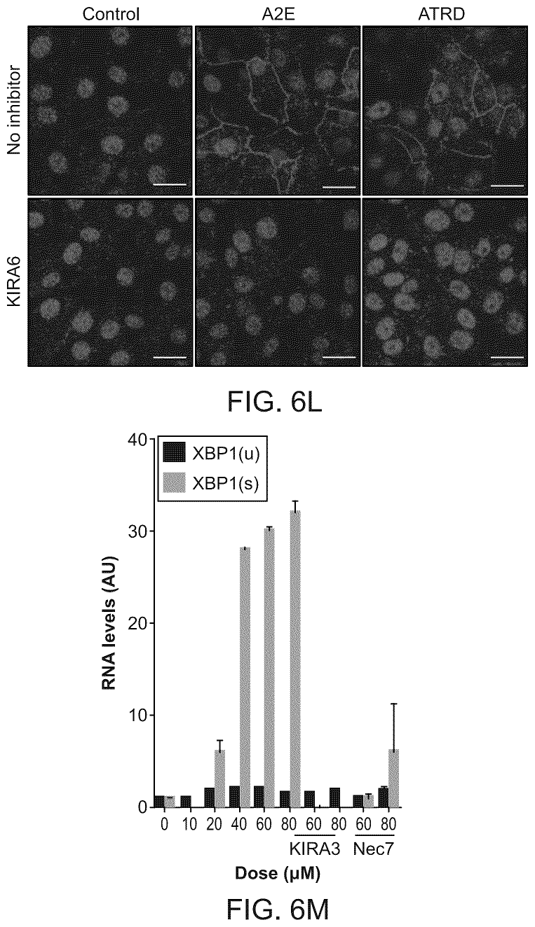

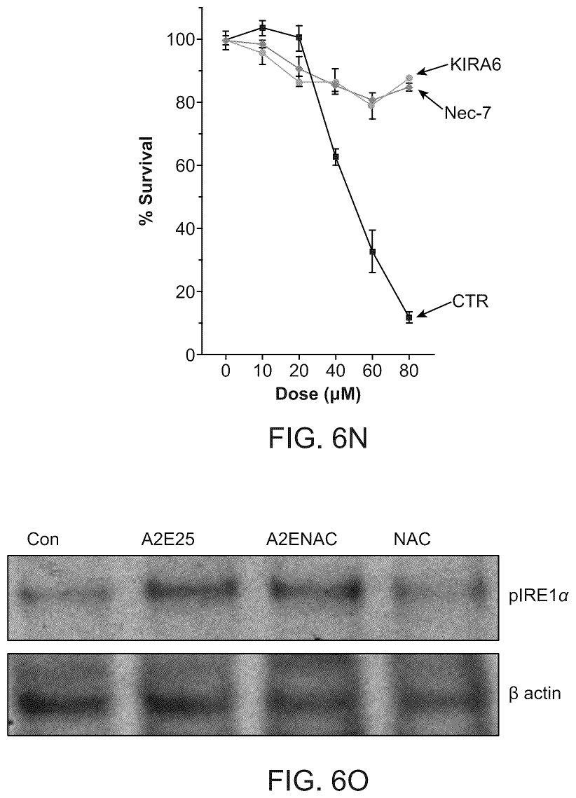

The causal link between LF accumulation and ER-stress without light assistance was analyzed (FIG. 6A). The PERK branch was significantly activated by LBs. Indeed, A2E produced the strongest Ser51 phosphorylation of eIF2a while ATRD the highest induction of ATF4 and BiP. qPCR confirmed the induction of ATF4 at the mRNA level, by both LBs (FIG. 6B). The activation of the IRE1α branch was visualized by qPCR. As shown in FIGS. 6C-6D, dose and time-dependent inductions of XBP1s by A2E and ATRD were observed in the dark.

Regular PCR coupled with agarose gel revealed splicing of XBP1 in ARPE19 and hfRPE (FIG. 6E). ATF6 also demonstrated significant cleavage after 6 hrs treatment with 20 μM A2E or tunicamycin (Tn) (FIG. 6F). Nec7 pretreatment completely abrogated the splicing of XBP1 (FIG. 6G) and the upregulation of CHOP (FIG. 6H), indicating robust inhibition of ER-stress. In contrast, cells incubated with A2E and treated with RIPK3 or RIPK1 inhibitors, exhibited normal CHOP upregulation in response to LF. Taken together, these results demonstrate that accumulation of LBs activate the three branches of UPR in the absence of illumination and Nec7 was capable of abrogating UPR.

To determine how UPR was implicated in LF induced necroptosis, each of the three endogenous ER-stress sensors/effectors, PERK, ATF6 and IRE1α, were individually knocked out (KO) in ARPE-19 cells, and their susceptibility to LBs was tested with our light-free cell-death assay. Remarkably, IRE1α-KO showed significant increase in survival to toxic doses of LBs (FIG. 6I). IRE1α is a bifunctional kinase/RNase that upon ER-stress initiates a concatenated chain of activation events, starting with its dimerization, kinase activation with autophosphorylation and culminating with the activation of its RNase function. IRE1α is a type-I ER-transmembrane multidomain protein with a sensing domain towards the ER lumen that in the presence of unfolded proteins or perturbed lipid composition clusters to promote its kinase activity that trans-autophosphorylate the molecule and via allosteric modulation, activates the RNase at the far end of its cytosolic region.

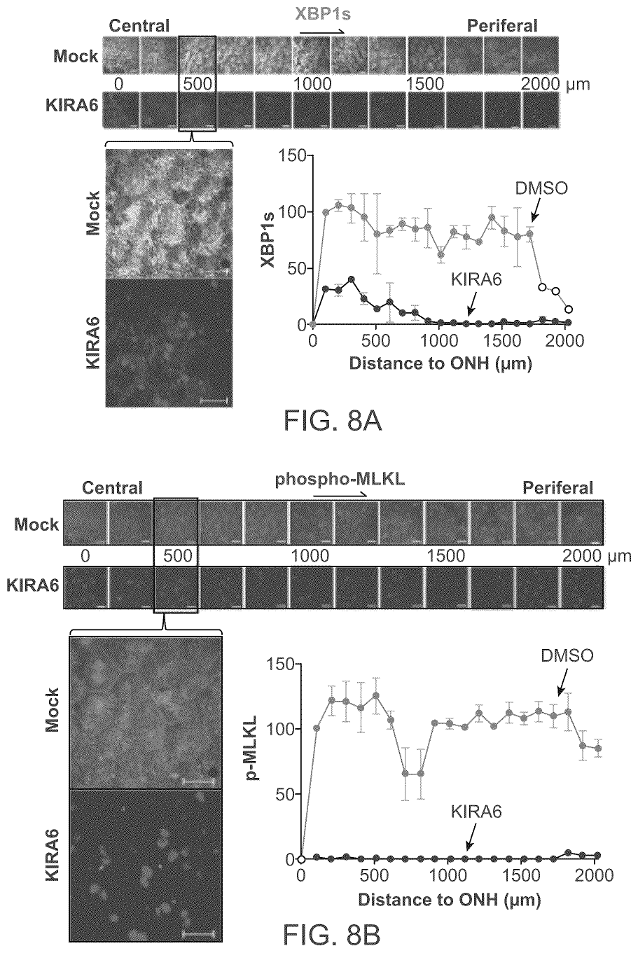

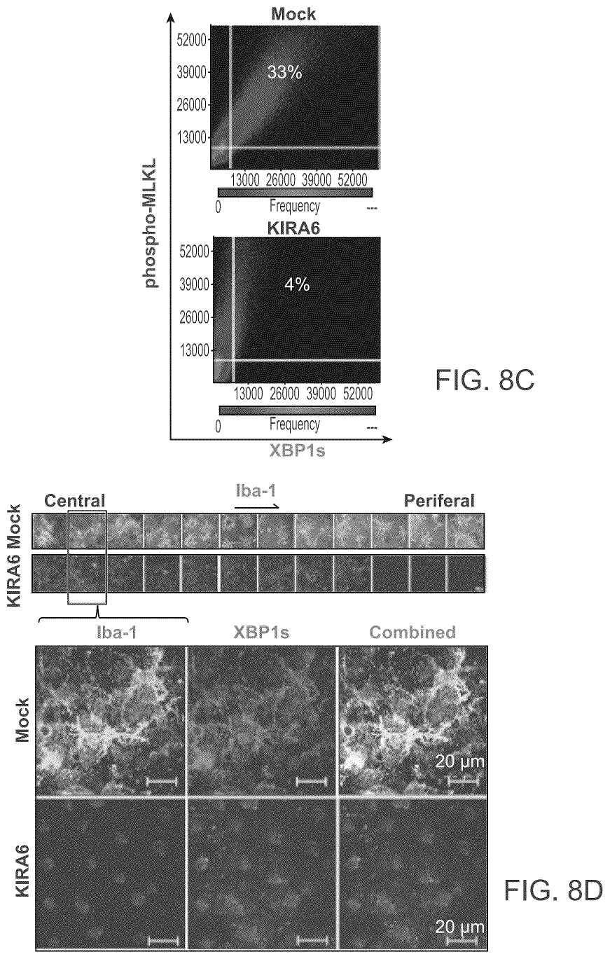

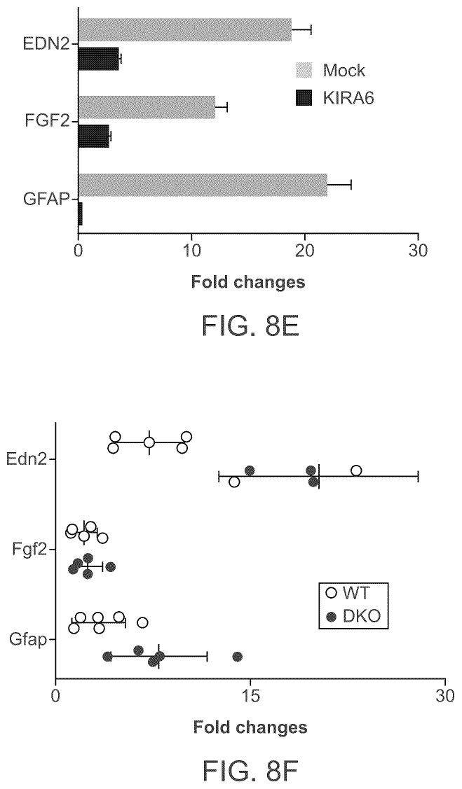

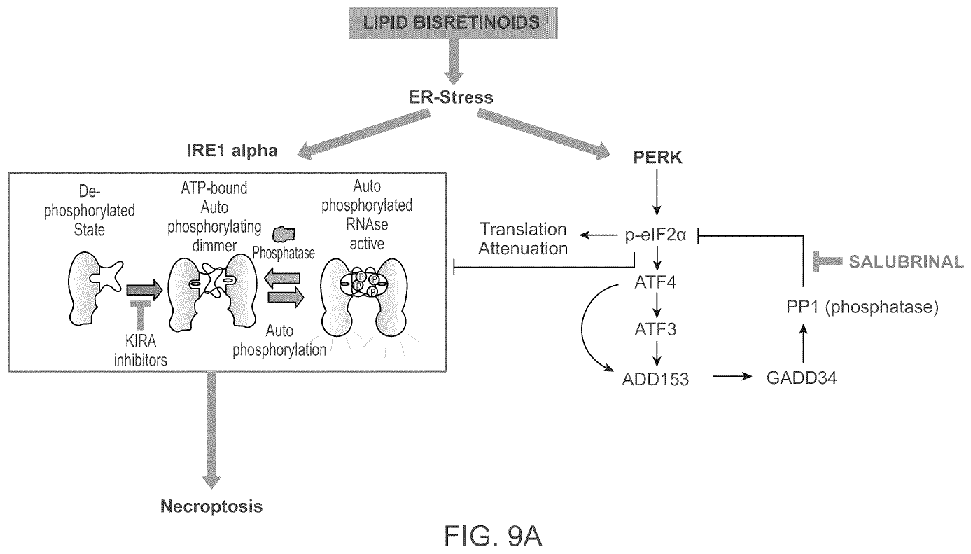

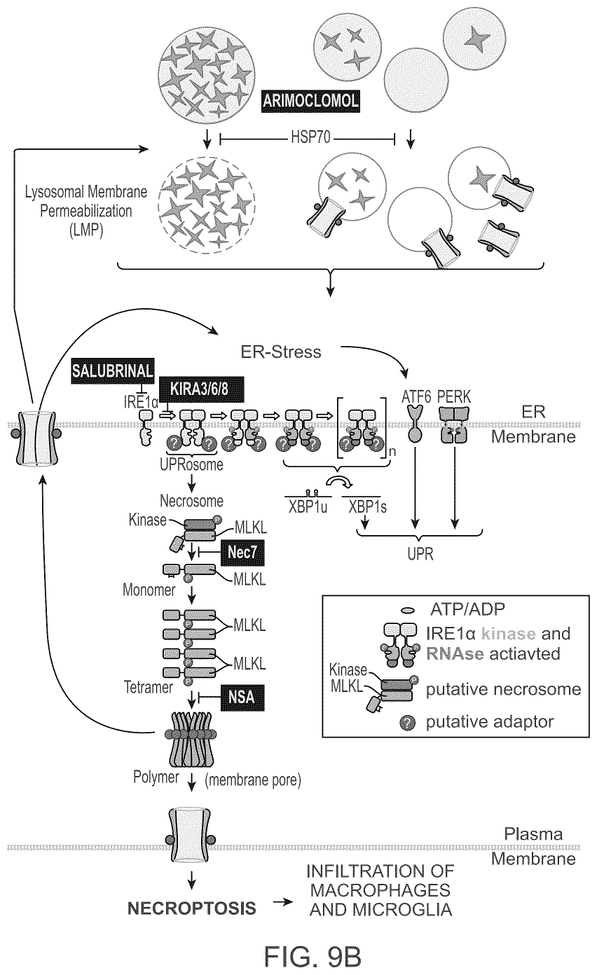

The critical role of IRE1α in the light-independent cell-death process was evaluated using small-molecules that selectively blocked IRE1α at the various stages of activation. A set of inhibitors selective for the kinase (APY29, sunitinib) or RNAse (STF-0831, 4μ8C, MKC-3946) functions were tested. Surprisingly, individual or combined inhibition of the kinase, RNAse or both activities did not translate into survival suggesting that LF-mediated necroptosis does not rely on the IRE1α canonical signaling pathway as the UPR response (FIG. 6J). In contrast, blocking dimerization (e.g., KIRA3, KIRA6) was sufficient to protect against necroptosis by LF (FIG. 6J). This result is consistent with the formation of UPRosomes, multimolecular structures where dimers or oligomers of IRE1α act as scaffolds where interacting proteins assemble to cross-circuit with other pathways and confer novel functional outputs (Urra H, et al. (2018) Nat Cell Biol 20(8):942-953; Hetz C, Chevet E, Oakes S A (2015) Nat Cell Biol 17(7):829-838; Petersen S L, et al. (2015) Cell Death Differ 22(11):1846-1857; Sepulveda D, et al. (2018) Mol Cell 69(2):238-252.e7; Hetz C, Glimcher L H (2009) Mol Cell 35(5):551-561). Thus, LF may induce the expression of an adaptor protein that assembles into IRE1α-UPRosomes and bridges ER-stress with necrosome formation (FIGS. 8A-8E; FIG. 9B).

These results demonstrate that Dabrafenib, necrosulfonamide (NSA), Necrostatin 7 (Nec7), Arimoclomol, and IRE1α inhibitors that block IRE1α dimerization are useful in methods for preventing or treating an eye disease associated with retinal cell lipofuscin-associated cytotoxicity in a subject in need thereof.

### Example 6: IRE1α-Mediated Necroptosis in Retinas with LF

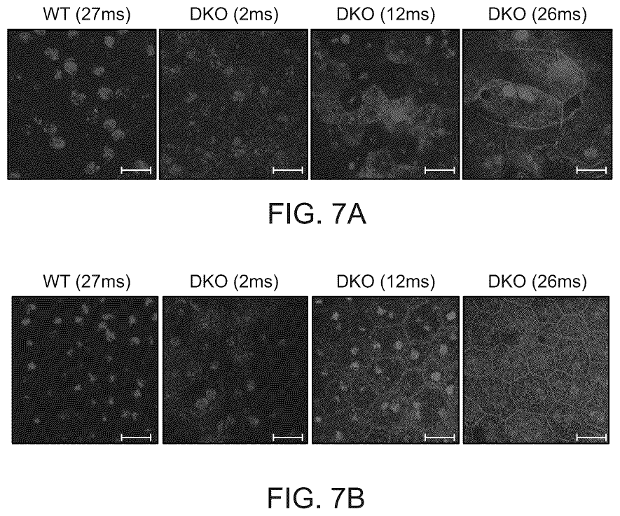

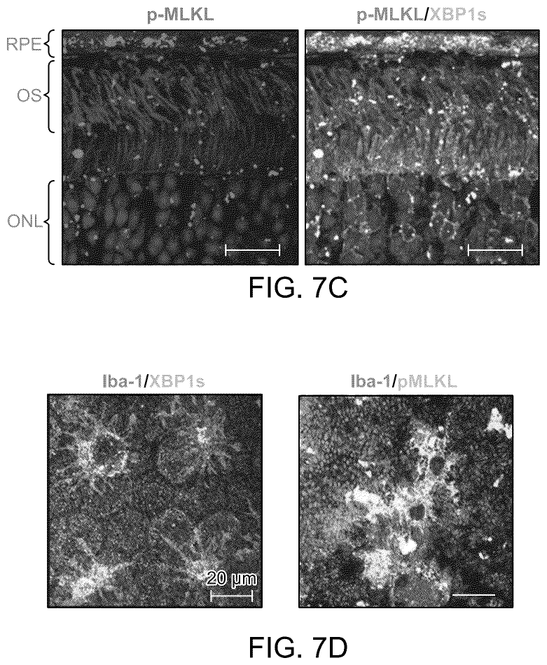

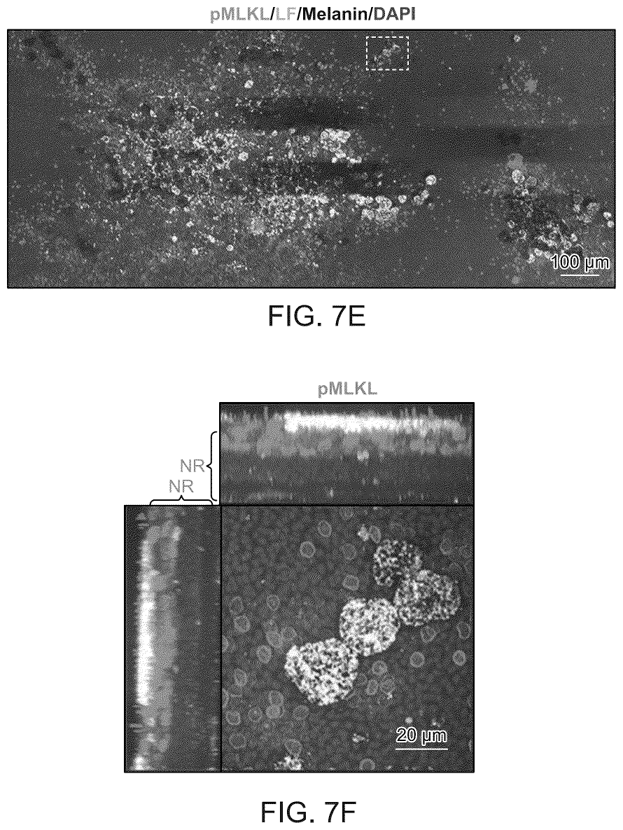

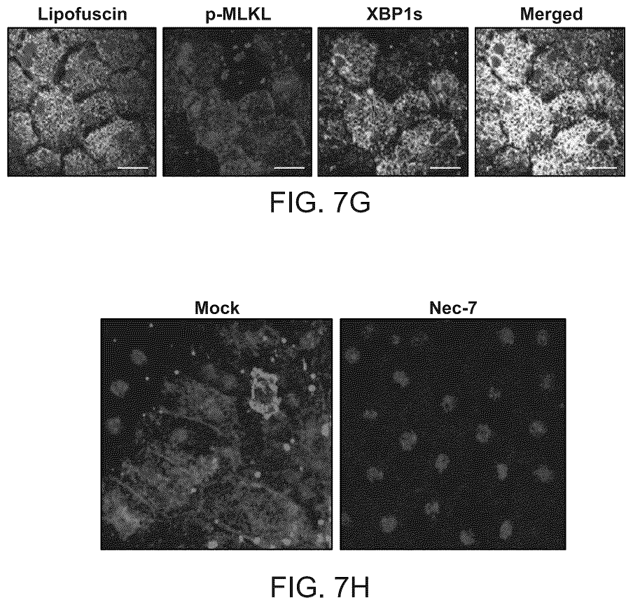

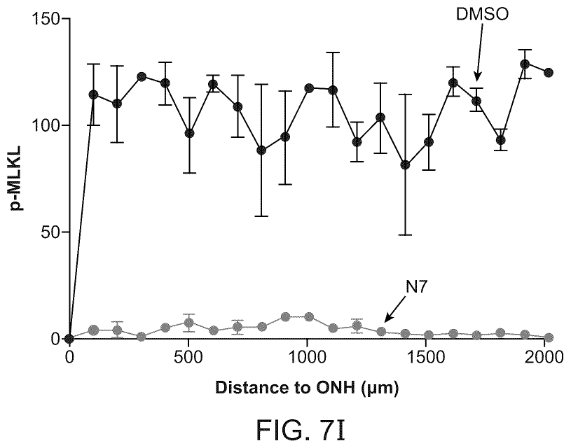

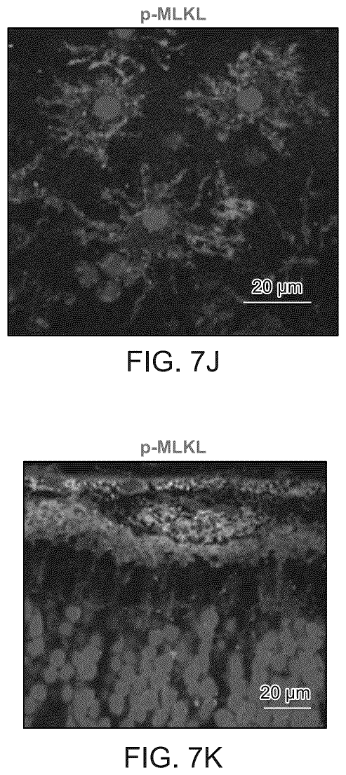

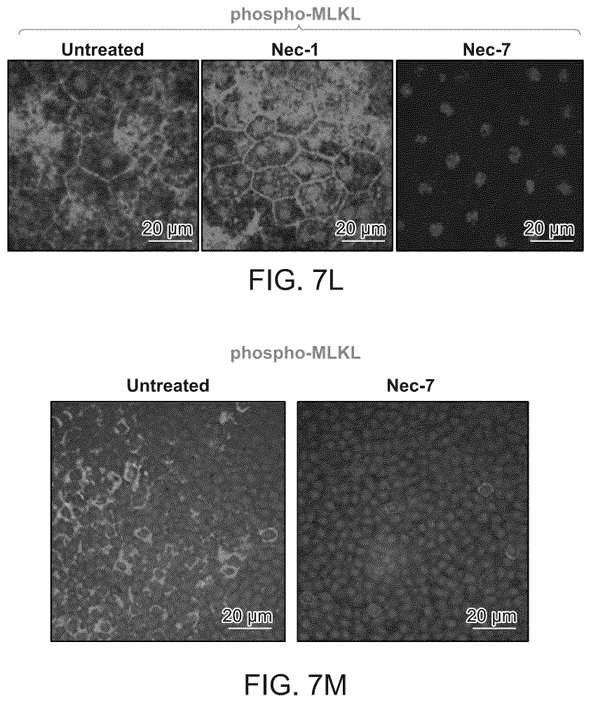

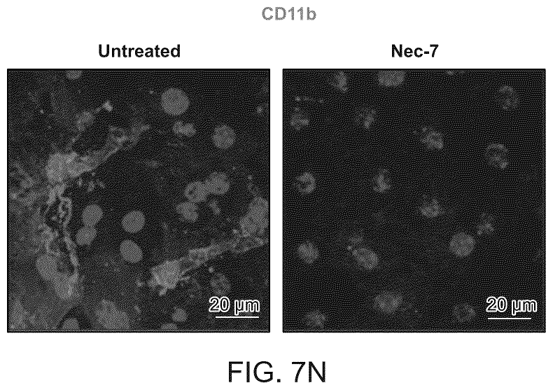

To investigate whether pigmented retinas affected with LF experience progressive IRE1α mediated deposition of pMLKL oligomers, eyes from 2, 12 and 27 months old ABCA4−/− RDH8−/− DKO and WT C57BL6 mice were dissected and their whole RPE, flat mounted and stained with anti-XBP1s (FIG. 7A) or anti phospho-Ser358 MLKL (FIG. 7B). Expression of XBP1s and pMLKL were barely detected in WT retinas, even in 27 months old mice. In contrast, DKO animals showed a diffused XBP1s staining at 2 months that by 1 year had increased in intensity in discrete clusters of RPE cells, and become widespread by 27 months. Similarly, pMLKL was dim at 2 months and became increasingly positive intracellularly first and in plasma membranes later (FIG. 7B). The staining for XBP1s and pMLKL became stronger and more widespread with age and pMLKL clearly showed association to cell membranes in the oldest DKO.

The atypical necroptosis observed in cell cultures was blocked by Nec7. To verify the role of Nec7 in in vivo detected necroptosis, 1 of vehicle and 1 of Nec7 was intraocularly injected in the right eyes and left eyes, respectively, of 12 month old DKOs and their pMLKL levels were analyzed one week later. As shown in FIG. 7H, membrane and cytosolic pMLKL labeling were reduced to undetectable levels post Nec7 treatment, confirming that the atypical necroptosis pathway is active in retinas with LF. In addition, the Nec7 treatment shows the specificity of the staining with pMLKL antibody. The reduction in pMLKL levels was complete throughout the retina (FIG. 7I). To characterize the distribution of ER-stress and necroptosis marker across the retina, dual staining for XBP1s and pMLKL on demelanized paraffin retinal cross-sections, from 20 month old DKOs was performed. The strongest pMLKL (FIG. 7C, left) and XBP1s (FIG. 7C, right) labels appeared in RPE and in the small migratory population LF+, Iba-1+, CD11b+ described before as microglia/macrophage with abundant lipofuscin content.

The phospho-MLKL staining was particularly evident for microglia/macrophages infiltrated into subretinal space that appeared attached to RPE in flat mounts from 20 months old DKO (FIG. 7J). Phospho-MLKL labeling in zones of RPE shedding large pieces of RPE cells showed particularly stronger staining around rather than in the lesion itself, suggesting the staining originated mainly from microglia/macrophages encapsulating the sloughed RPE cells (FIG. 7K).

XBP1s was also positive around the bodies, inner segments and large parts of the outer segments of PRs. To further confirm the activated status of the microglia, the phenotype of Iba-1+ cells infiltrated in the subretinal space was analyzed. RPE flat-mounts from 25 to 27 month DKOs were prepared and dual stained with Iba-1/XBP1s or Iba-1/pMLKL (FIG. 7D). Iba-1+ cells displayed activated morphology and stained positive for ER-stress and necroptosis markers.

Very impressive was the discovery of a vast phospho-MLKL staining around the zones of the neural retina, where RPE cells had migrated (FIGS. 7E-7F) demonstrating the induction of a halo of necroptosis in the surrounding areas. It could represent adjacent photoreceptor cells undergoing necroptosis, or the dispersal of neuro-destructive activated macrophages/microglia piggy-bagged by the migrating RPE. In either case, the intense phospho-MLKL staining appears to precede and correlate well with the exacerbated loss of photoreceptors in the areas of the ONL bordering migratory RPEs, as shown in FIGS. 2C and 2H.

To establish the therapeutic potential of inhibiting the atypical necroptosis pathway, 2 μl of necrostatins, or vehicle were injected intraocularly in the eyes of 26-month-old DKO mice. A week later, the status of their retinas was evaluated by staining RPE- and neuroretina-flat-mounts with anti phospho-MLKL antibody. It was verified that Nec7 but not Nec1, reduced membrane and cytosolic phospho-MLKL staining to undetectable levels (FIG. 7L), supporting the notion that the atypical necroptosis pathway detected in our in vitro system was active in retinas loaded with lipofuscin. FIG. 7I depicts the phospho-MLKL mean fluorescence expression values, of four vehicles and four Nec7 treated eyes, measured every 0.1 mm intervals from the ONH in RPE-flat mounted inferior hemiretinas from 24 months-old DKO. The staining became negative from the center to the periphery in Nec7 treated eyes, while remained strongly positive in the companion mock treated eyes. To analyze the impact of treatment on photoreceptors, phospho-MLKL expression in neuroretina-flat mounts from control and Nec7 treated eyes was compared. The necroptosis labeling was significantly reduced by Nec7 (FIG. 7M). Similarly, Nec7 reduced the infiltration of CD11b cells in the subretinal space, as shown by the reduction of macrophages/microglia on RPE-flat mounts from 25- to 27-month-old DKO mice (FIG. 7N). In summary, the results show that blockage of necroptosis with Nec7 significantly reduces the signs of retinal degeneration. On the basis of these observations, a model that summarizes these data and explains the mechanism underlaying light-independent lipofuscin cytotoxicity was generated (FIG. 9B). According to this working model, lipofuscin accumulation causes LMP that elicits the assembly of an atypical necrosome which in turn mediates MLKL phosphorylation/polymerization. Phospho-MLKL-oligomeric pores would progressively insert into cellular membranes of lysosomes and plasma membrane, causing more LMP, which in turn promotes more phospho-MLKL deposition on cell membranes. This creates a vicious loop until the number of phospho-HLKL pores per cell is such that the cell undergoes a necroptotic break down.

These results demonstrate that Dabrafenib, necrosulfonamide (NSA), Necrostatin 7 (Nec7), Arimoclomol, and IRE1α inhibitors that block IRE1α dimerization are useful in methods for preventing or treating an eye disease associated with retinal cell lipofuscin-associated cytotoxicity in a subject in need thereof.

### Example 7: Efficacy of IRE1α Inhibition in Retinas with LF

To establish the therapeutic potential of inhibiting IRE1α-driven cell-death for pigmented retinas with LF accumulation, the IRE1α inhibitor, KIRA6, or vehicle was injected intraocularly in the eyes of 26 month old DKO mice. A week later, their retinas status was evaluated by staining whole RPE flat mounts with specific antibodies. XBP1s staining was decreased from the center to the periphery by the KIRA6 treatment compared to mock treated eyes. These results demonstrate that KIRA6 was effective at blocking IRE1α signals in vivo and that the XBP1s detection was specific (FIG. 8A).

Furthermore, KIRA6 abrogated necroptosis, as depicted by the center to periphery disappearance of intracellular and plasma membrane labelling by pMLKL antibody (FIG. 8B). Comparative analysis of dual color dot-plots, pMLKL vs XBP1s, obtained by confocal microscopy of demelanized paraffin cross-sections, from 20 month old DKOs, show a reduction, from 33% to 4%, in double positive cells, i.e. RPE and microglia cells, by the IRE1α inhibitor (FIG. 8C). To determine if the reduction in double positive microglia was caused by diminished infiltration of Iba1+ cells or downregulated expression of XBP1s/pMLKL markers, RPE-flat mounts from KIRA6 and mock treated eyes were stained with Iba-1 and XBP1s antibodies. No Iba-1+ cells were detected attached to the apical side of RPE in successive images taken from the center to the periphery of the retina, indicating the KIRA6 treatment served to reduce infiltration of activated microglia and consequently to reduce the inflammation of the retina (FIG. 8D). To verify reduction in retinal degeneration (EDN2, FGF2), inflammation/angiogenesis (GFAP, SERP, VEGF, CXCL15), ER-stress (XBP1s, SCAND1, CEBPA) and necroptosis (HMGA), total RNA from the whole retina (neuroretina plus RPE) of KIRA6 and control injected eyes was isolated, and mRNA levels were quantified by qPCR. The results indicated a generalized reduction in all the tested markers for retinal degeneration, inflammation/angiogenesis, ER-stress and necroptosis (FIG. 8E).

Collectively, these results demonstrate that ER-stress and necroptosis were exacerbated in the retinas with LF. Treatment with Nec7 reduced pMLKL staining indicating the labeling was specific and that necroptosis in the retinas was inhibited with Nec7, as in RPE cultures. Histology of retinas with high content of LF revealed that IRE1α and pMLKL membrane deposition were increasingly present in RPE and invading microglia and that both IRE1α and pMLKL tend to colocalize in the same cells as LF-driven degeneration proceeded. The type of necroptosis found in the retinas was susceptible to inhibition with Nec7 suggesting it represented the same type of atypical necroptosis observed in cultured cells. Treatment with KIRA6 normalized the levels of IRE1α activation, pMLKL oligomerization and Iba1+ microglia infiltration as well as multiple markers of ongoing retinal degeneration detected by qPCR (FIG. 8E). Staining of retinal cross-sections revealed not only RPE but also microglia (CD11b+, Iba-1+) LF+ cells were positive for XBP1s and phospho-MLKL Ser345. Treatment with KIRA6 eliminated XBP1s and phospho-MLKL Ser345 labelling from all cell types.

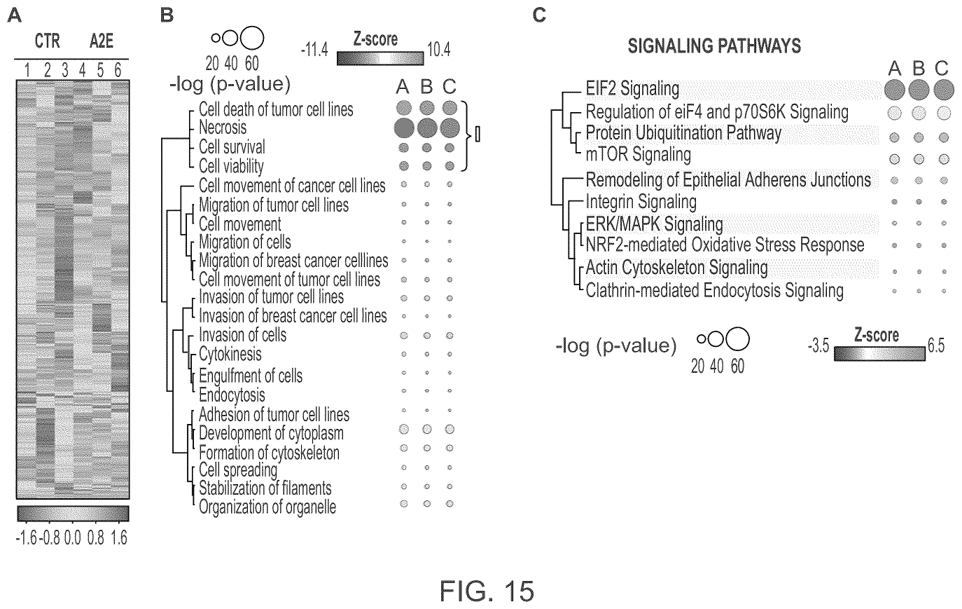

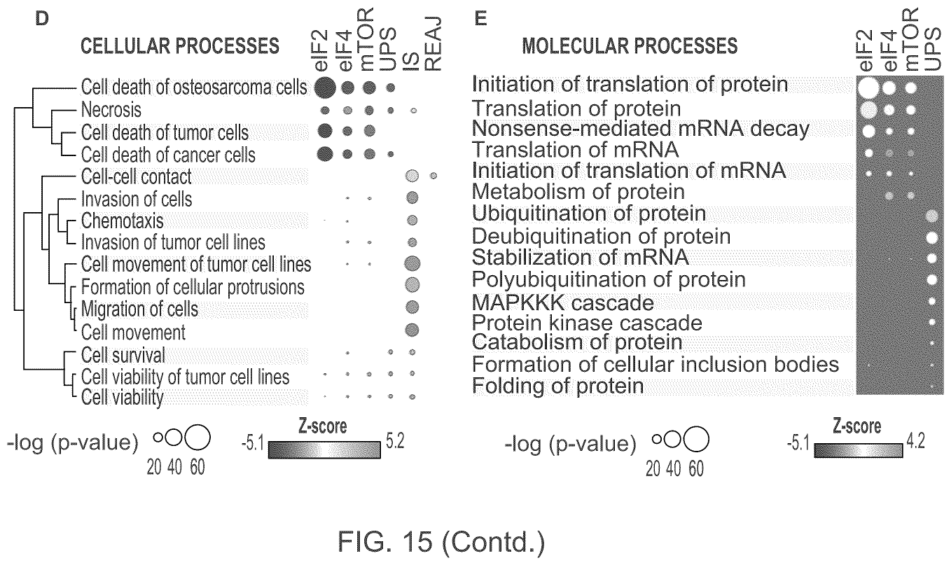

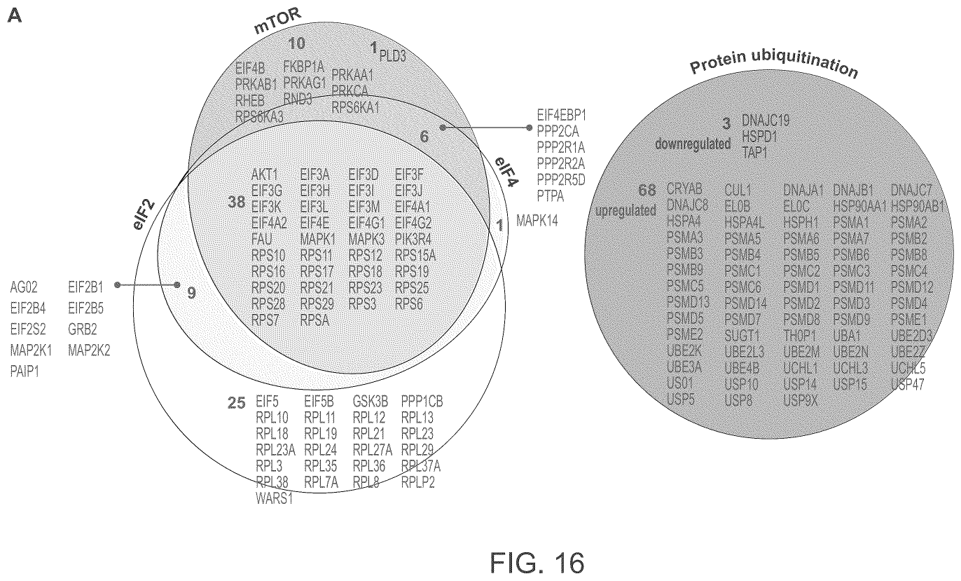

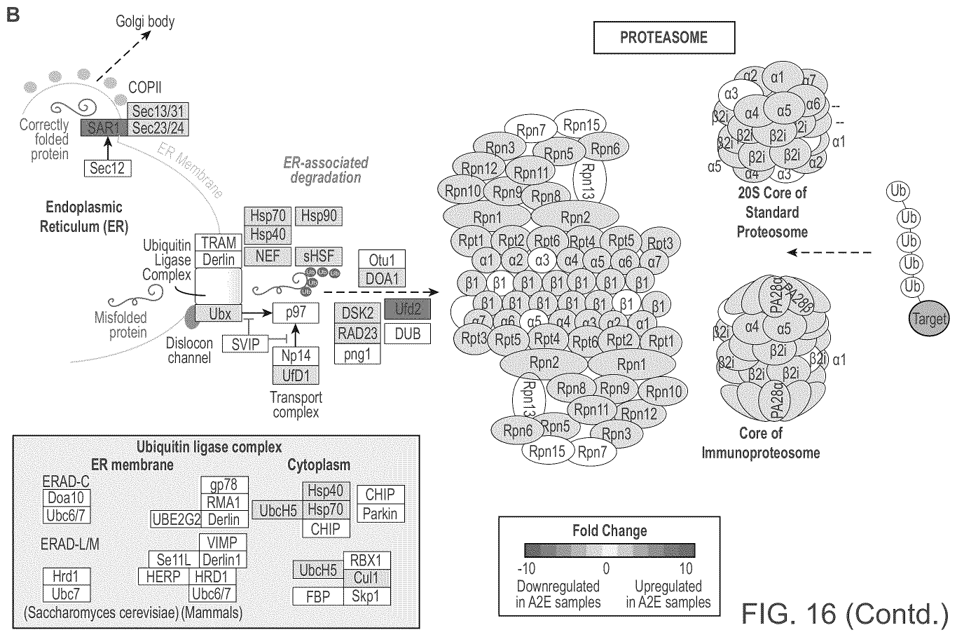

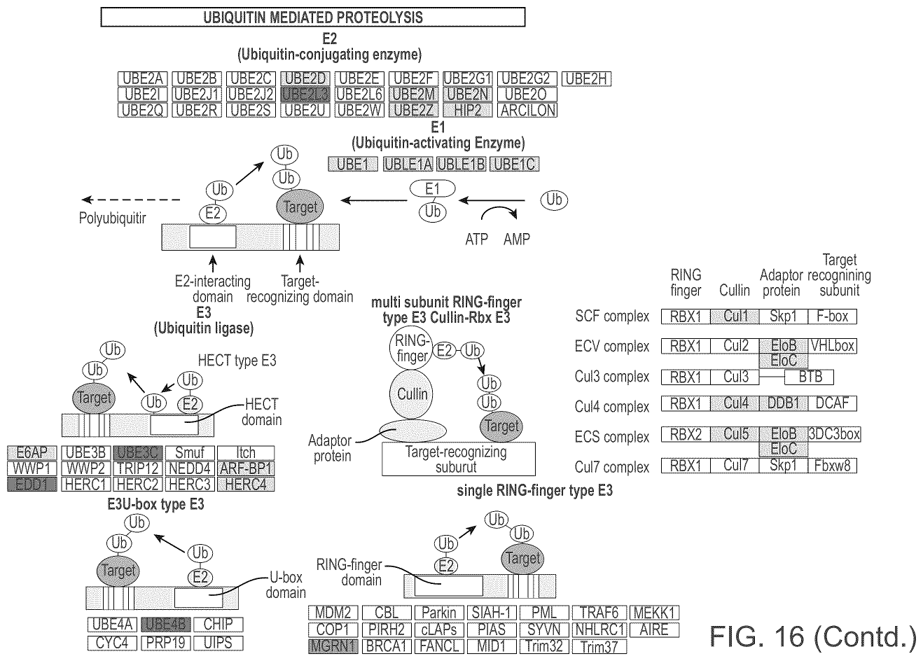

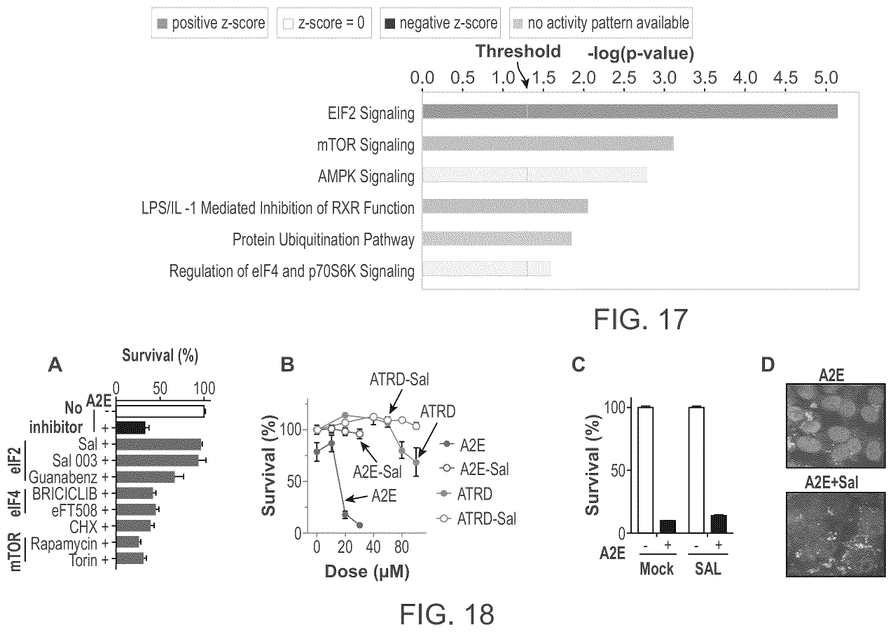

FIGS. 15A-15E and FIGS. 16A-16B show a comparative proteomic analysis between ARPE-19 cells with and without lipofuscin, and display the proteins modulated in RPE cells to survive lipofuscin accumulation. As shown in FIG. 17, the top anti-necroptotic pathways, identified by proteomic methods in cultured cells, eIF2a, eIF4, mTOR, and UPS, appear to be increased along with lipofuscin in the RPE of eyes of ABCA4−/−RDH8−/− double knockout (DKO) mice. The protective effects of inducers of eIF2a, eIF4 or mTOR pathways against lethal amounts of lipofuscin were evaluated.

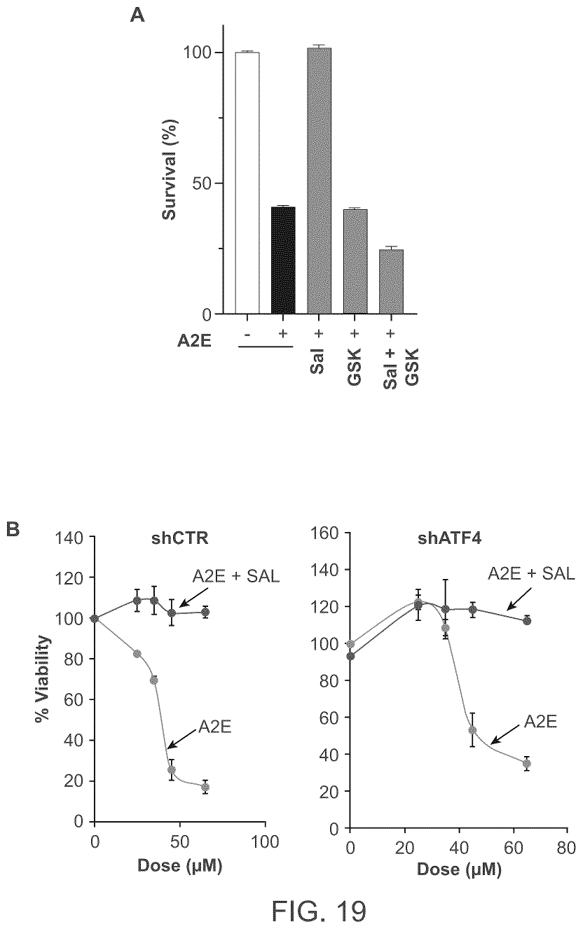

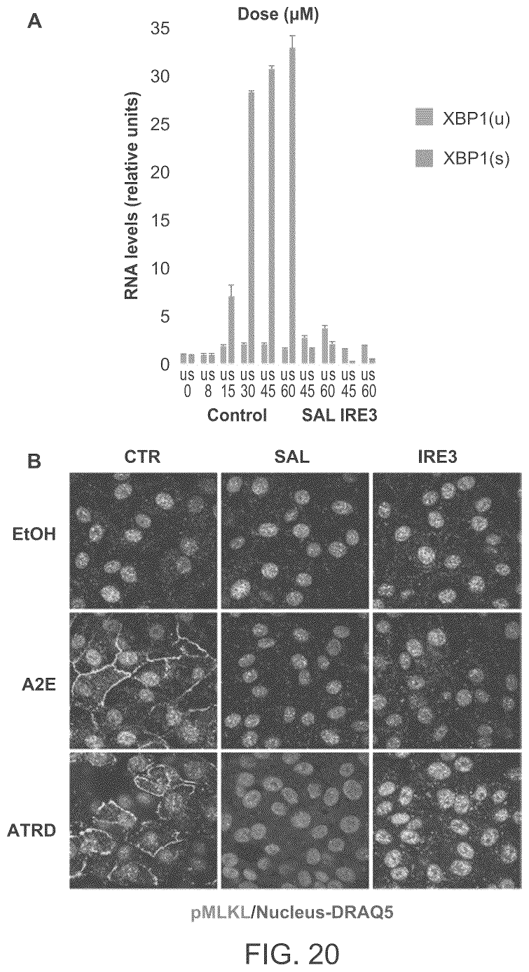

Only Salubrinal (SAL) and SAL003, that targeted both cellular eIF2a phosphatases comprised of PP1 bound to either GADD34 or CreP, catalytic subunits, protected against lipofuscin. In contrast, Guanabenz, that only disrupts PP1-GADD34 association or eIF4 and mTOR activators, did not confer significant protection (see FIGS. 18A-18B). As such, not all inhibitors of eIF2a, eIF4 or mTOR pathways are capable of conferring protection against lipofuscin cytotoxicity. FIGS. 18C-18D show that SAL does not protect against the phototoxic decomposition of lipid bisretinoids, but is able to protect cells even if they contain large amounts of lipofuscin in their cytosol. FIGS. 19A-19B show SAL needs PERK but not ATF4 to exert protection against lipofuscin. SAL inhibits IRE1α signaling and thus prevents necroptosis of RPE by lipofuscin. Knockdown of IRE1α but not PERK or ATF6 (the three sensors of ER-stress) prevents necroptosis by lipofuscin. See FIGS. 20A-20B.

These results demonstrate that Dabrafenib, necrosulfonamide (NSA), Necrostatin 7 (Nec7), Salubrinal, SAL003, Arimoclomol, and IRE1α inhibitors that block IRE1α dimerization are useful in methods for preventing or treating an eye disease associated with retinal cell lipofuscin-associated cytotoxicity in a subject in need thereof.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all FIG.s and tables, to the extent they are not inconsistent with the explicit teachings of this specification.

1. A method for preventing or treating an eye disease associated with retinal cell lipofuscin-associated cytotoxicity in a subject in need thereof comprising administering to the subject an effective amount of at least one therapeutic agent selected from the group consisting of dabrafenib, necrosulfonamide (NSA), arimoclomol, a Kinase Inhibiting RNase Attenuator (KIRA) compound, salubrinal, SAL003 and any pharmaceutically acceptable salt thereof, wherein the eye disease associated with retinal cell lipofuscin-associated cytotoxicity is autosomal recessive retinitis pigmentosa (RP), Stargardt disease (STGD), Best disease (BD), cone-rod dystrophy, or ABCA4 mutant Age-Related Macular Degeneration (AMD).

2. The method of claim 1, wherein the KIRA compound is KIRA3, KIRA6, KIRA7, or KIRA8.

3. The method of claim 1, wherein the subject comprises a mutation in ABCA4 and/or RDH12, optionally wherein the mutation in ABCA4 and/or RDH12 is homozygous or heterozygous.

4. (canceled)

5. The method of claim 1, wherein administration of the effective amount of the at least one therapeutic agent prevents exacerbation of lipofuscin-associated cytotoxicity in retinal cells in the subject.

6. A method for preventing or treating an ABCA4 mutant eye disease associated with retinal cell lipofuscin-associated cytotoxicity in a subject in need thereof comprising administering to the subject an effective amount of Necrostatin 7 (Nec7) or a pharmaceutically acceptable salt thereof, wherein the ABCA4 mutant eye disease associated with retinal cell lipofuscin-associated cytotoxicity is autosomal recessive retinitis pigmentosa (RP), cone-rod dystrophy, or Age-Related Macular Degeneration (AMD).

7. The method of claim 6, wherein administration of the effective amount of Nec7 or pharmaceutically acceptable salt thereof prevents exacerbation of lipofuscin-associated cytotoxicity in retinal cells in the subject.

8. The method of claim 1, wherein the eye disease is genetic, non-genetic, or associated with aging.

9. The method of claim 1, wherein the AMD is dry AMD.

10. The method of claim 1, wherein the cone-rod dystrophy is autosomal recessive cone-rod dystrophy.

11. The method of claim 1, wherein the subject harbors at least one ABCA4 mutation selected from the group consisting of ABCA4 D2177N, ABCA4 G1961E, ABCA4 G863A, ABCA4 1847delA, ABCA4 L541P, ABCA4 T2028I, ABCA4 N247I, ABCA4 E1122K, ABCA4 W499*, ABCA4 A1773V, ABCA4 H55R, ABCA4 A1038V, ABCA4 IVS30+1G→T, ABCA4 IVS40+5G→A, ABCA4 IVS14+1G→C, and ABCA4 F1440del1cT.

12. The method of claim 1, wherein the subject harbors at least one RDH12 mutation selected from the group consisting of RDH12 G127*, RDH12 Q189*, RDH12 Y226C, RDH12 A269Gfs*, RDH12 L274P, RDH12 R65*, RDH12 H151D, RDH12 T155I, RDH12 V41L, RDH12 R314W and RDH12 V146D.

13. The method of claim 1, wherein dabrafenib, NSA, arimoclomol, the KIRA compound, salubrinal, SAL003, Nec7, or the pharmaceutically acceptable salt thereof is administered via topical, intravitreous, intraocular, subretinal, or subscleral administration.

14. The method of claim 1, wherein dabrafenib, NSA, arimoclomol, the KIRA compound, salubrinal, SAL003, Nec7, or the pharmaceutically acceptable salt thereof reduces or eliminates lipofuscin bisretinoid (LB) lipid-induced phosphorylation and/or polymerization of MLKL, optionally wherein the LB lipids are selected from the group consisting of N-retinylidene-N-retinylethanolamine (A2E), an A2E isomer, an oxidized derivative of A2E, and all-trans-retinal dimers (ATRD).

15. The method of claim 1, wherein dabrafenib, NSA, arimoclomol, the KIRA compound, salubrinal, SAL003, Nec7, or the pharmaceutically acceptable salt thereof reverses LB lipid-induced translocation of phosphorylated MLKL (pMLKL) to plasma membrane in retinal pigment epithelium cells, optionally wherein the LB lipids are selected from the group consisting of N-retinylidene-N-retinylethanolamine (A2E), an A2E isomer, an oxidized derivative of A2E, and all-trans-retinal dimers (ATRD).

16. (canceled)

17. The method of claim 1, wherein dabrafenib, NSA, arimoclomol, the KIRA compound, salubrinal, SAL003, Nec7, or the pharmaceutically acceptable salt thereof reduces mRNA or protein levels of one or more genes associated with retinal degeneration, inflammation/angiogenesis, ER-stress and/or necroptosis.

18. The method of claim 17, wherein the one or more genes associated with retinal degeneration, inflammation/angiogenesis, ER-stress and/or necroptosis are selected from the group consisting of EDN2, FGF2, GFAP, SERP, VEGF, CXCL15, XBP1s, SCAND1, CEBPA and HMGA.

19. The method of claim 1, wherein dabrafenib, NSA, arimoclomol, the KIRA compound, salubrinal, SAL003, Nec7, or the pharmaceutically acceptable salt thereof inhibits or mitigates lipofuscin-induced necroptosis.

20. The method of claim 1, wherein dabrafenib, NSA, arimoclomol, the KIRA compound, salubrinal, SAL003, Nec7, or the pharmaceutically acceptable salt thereof reduces infiltration of activated microglia/macrophage in retinal pigment epithelium cells.

21. The method of claim 1, wherein dabrafenib, NSA, arimoclomol, the KIRA compound, salubrinal, SAL003, Nec7, or the pharmaceutically acceptable salt thereof is conjugated to an agent that targets retinal pigment epithelium cells.

22. The method of claim 21, wherein the agent that targets retinal pigment epithelium cells is tamoxifen, chloroquine (CQ)/hydroxychloroquine (HCQ), ethambutol (EMB), or sodium iodate (NaIO₃).


SEQUENCE LISTING (12 sequences)

SEQ ID NO: 1 - DNA, 20 bp, Artificial Sequence
  Description of Artificial Sequence Synthetic primer
gctgagtccgcagcaggtgc

SEQ ID NO: 2 - DNA, 22 bp, Artificial Sequence
  Description of Artificial Sequence Synthetic primer
gggtccaasttgtccagaatgc

SEQ ID NO: 3 - DNA, 20 bp, Artificial Sequence
  Description of Artificial Sequence Synthetic primer
gctgagtccgcagcactcag

SEQ ID NO: 4 - DNA, 22 bp, Artificial Sequence
  Description of Artificial Sequence Synthetic primer
gggtccaasttgtccagaatgc

SEQ ID NO: 5 - DNA, 20 bp, Artificial Sequence
  Description of Artificial Sequence Synthetic primer
tgtggatgggttggtcagtc

SEQ ID NO: 6 - DNA, 20 bp, Artificial Sequence
  Description of Artificial Sequence Synthetic primer
tcagaaggtcatctggcatg

SEQ ID NO: 7 - DNA, 20 bp, Artificial Sequence
  Description of Artificial Sequence Synthetic primer
cacctgcgttttcgtcgatg

SEQ ID NO: 8 - DNA, 21 bp, Artificial Sequence
  Description of Artificial Sequence Synthetic primer
ccagtgtcttcgatggcagaa

SEQ ID NO: 9 - DNA, 20 bp, Artificial Sequence
  Description of Artificial Sequence Synthetic primer
gcgacccacacgtcaaacta

SEQ ID NO: 10 - DNA, 23 bp, Artificial Sequence
  Description of Artificial Sequence Synthetic primer
tcccttgatagacacaactcctc

SEQ ID NO: 11 - DNA, 20 bp, Artificial Sequence
  Description of Artificial Sequence Synthetic primer
gcgacccacacgtcaaacta

SEQ ID NO: 12 - DNA, 23 bp, Artificial Sequence
  Description of Artificial Sequence Synthetic primer
tcccttgatagacacaactcctc